SEQUENCE LISTING (60 sequences)

SEQ ID NO: 1 - DNA, 780 bp, Oryza sativa
atgtcgacggcggtggcggacgtgccaccggcggcggcctacgggttccccggatcggccaagagagggaagcctgaggaggtggtggtgctgatggggaagaggaggaacgaagggttcttcatcgaggaggaggaggaggaggaggaggtgctgacggagagctcgtcgatcggcgcgccgtcgccggcgagctcgtcgatcggggagaactccggcgaggaggagggaggggacgacgaggaggaggtggagagcaagctcaaggcggaggatgagcaggtcggcctcggctgcttggacgccttggaggaatccttacccatcaagagggggctctccaacttctacgccggcaagtccaagtcgttcaccagcctcgccgaggcgacggcgtcgccggcggcggcggccaacgagctggccaagccggagaaccccttcaacaagcgccgccgcatcctcgccacctggtcgcggcgagcctcctgcagctccctcgccaccgccacctacctcccacctctcctcgcgcccgaccacgccgtcgccgagggcgacgagggtgaggaggaagacgacgattcagacgacgatgagcggcagcaccgtggcaagaacggcggccggcgagagtcggcggcgccgccattgccattgccgccgccgaggctcaccttgcacacccagatgggaggaatggtgaggaggaatggaacattcaggtcgccgaggtcgctctcactgtctgatcttcagaacagtggcggttcatgttag

SEQ ID NO: 2 - protein, 259 aa, Oryza sativa
MSTAVADVPPAAAYGFPGSAKRGKPEEVVVLMGKRRNEGFFIEEEEEEEEVLTESSSIGAPSPASSSIGENSGEEEGGDDEEEVESKLKAEDEQVGLGCLDALEESLPIKRGLSNFYAGKSKSFTSLAEATASPAAAANELAKPENPFNKRRRILATWSRRASCSSLATATYLPPLLAPDHAVAEGDEGEEEDDDSDDDERQHRGKNGGRRESAAPPLPLPPPRLTLHTQMGGMVRRNGTFRSPRSLSLSDLQNSGGSC

SEQ ID NO: 3 - DNA, 786 bp, Oryza sativa
atgtccacggcggtggcgcgcggcgggatgatgccggcggggcacgggttcgggaaggggaaggcggcggcggtggaggaggaggaggatgaggtgaacgggttcttcgtggaggaggaggaggaggaggaggaggaggaggaggcggcggtgtcggatgcgtcgtcgatcggggcggcgtcgtcggacagctcgtcgatcggggagaactcgtcgtcggagaaggagggggaggaggagggggaggaggtggagagcaaggcgaaggaggtggcggtggaggtggagggaggggggctcgggttccatggattggggactctcgaatccctggaggacgcccttcccatcaagaggggactctccaacttctacgccggcaagtccaagtcgttcacgagcctggccgaggcggcggcgaaggcggcggcgaaggagatcgccaagccggagaacccgttcaacaagcgccgccgcgtcctcgccgcgtggtcgcggcggcgcgcgtcctgcagctcgctggccaccacctacctgccccctctcctcgcccccgaccacgccgtcgtcgaggaggaggacgaggaggacgactccgacgccgagcagtgcagcggcagcggcggcggcaaccgccggcgcgagccgacgttcccgccgccgaggctgagcctgcacgcgcagaagagcagcttgacgccgaggagctcgaatccggcgtcgtcgtttagatctcctaggtcattctcactatccgatctccaaaatgcaggctcctataactag

SEQ ID NO: 4 - protein, 261 aa, Oryza sativa
MSTAVARGGMMPAGHGFGKGKAAAVEEEEDEVNGFFVEEEEEEEEEEEAAVSDASSIGAASSDSSSIGENSSSEKEGEEEGEEVESKAKEVAVEVEGGGLGFHGLGTLESLEDALPIKRGLSNFYAGKSKSFTSLAEAAAKAAAKEIAKPENPFNKRRRVLAAWSRRRASCSSLATTYLPPLLAPDHAVVEEEDEEDDSDAEQCSGSGGGNRRREPTFPPPRLSLHAQKSSLTPRSSNPASSFRSPRSFSLSDLQNAGSYN

SEQ ID NO: 5 - DNA, 24 bp, Artificial Sequence
  primer
caccatgtcgacggcggtggcgga

SEQ ID NO: 6 - DNA, 17 bp, Artificial Sequence
  primer
acatgaaccgccactgt

SEQ ID NO: 7 - DNA, 20 bp, Artificial Sequence
  primer
gctattagtacaaaaaaaat

SEQ ID NO: 8 - DNA, 24 bp, Artificial Sequence
  primer
cacctcaaaagcaaagcaattgac

SEQ ID NO: 9 - DNA, 24 bp, Artificial Sequence
  primer
caccgtggagagcaagctcaaggc

SEQ ID NO: 10 - DNA, 20 bp, Artificial Sequence
  primer
ctcctcctcgtcgtcccctc

SEQ ID NO: 11 - DNA, 20 bp, Artificial Sequence
  primer
cgaccaggtggcgaggatgc

SEQ ID NO: 12 - DNA, 24 bp, Artificial Sequence
  primer
cacccggcgagcctcctgcagctc

SEQ ID NO: 13 - DNA, 32 bp, Artificial Sequence
  primer
tgctgctgcggcgtagaagttggagagccccc

SEQ ID NO: 14 - DNA, 32 bp, Artificial Sequence
  primer
gcagcagcaaccagcctcgccgaggcgacggc

SEQ ID NO: 15 - DNA, 25 bp, Artificial Sequence
  primer
ggcgtagaagttggagagccccctc

SEQ ID NO: 16 - DNA, 25 bp, Artificial Sequence
  primer
accagcctcgccgaggcgacggcgt

SEQ ID NO: 17 - DNA, 23 bp, Artificial Sequence
  primer
ggccgcgttgaaggggttctccg

SEQ ID NO: 18 - DNA, 25 bp, Artificial Sequence
  primer
gccgccatcctcgccacctggtcgc

SEQ ID NO: 19 - DNA, 24 bp, Artificial Sequence
  primer
caccatgtccacggcggtggcgcg

SEQ ID NO: 20 - DNA, 20 bp, Artificial Sequence
  primer
gttataggagcctgcatttt

SEQ ID NO: 21 - DNA, 20 bp, Artificial Sequence
  primer
aaaatctaggtttctgcttc

SEQ ID NO: 22 - DNA, 24 bp, Artificial Sequence
  primer
caccgattcatcatcatcatcaag

SEQ ID NO: 23 - DNA, 25 bp, Artificial Sequence
  primer
ctccacctcctccccctcctcctcc

SEQ ID NO: 24 - DNA, 20 bp, Artificial Sequence
  primer
caccagcaaggcgaaggagg

SEQ ID NO: 25 - DNA, 30 bp, Artificial Sequence
  primer
gccgcccgtcctcgccgcgtggtcgcggcg

SEQ ID NO: 26 - DNA, 29 bp, Artificial Sequence
  primer
cgccgcgttgaacgggttctccggcttgg

SEQ ID NO: 27 - DNA, 25 bp, Artificial Sequence
  primer
caccatggagggagctggcagagat

SEQ ID NO: 28 - DNA, 18 bp, Artificial Sequence
  primer
aaggactctcagctgagt

SEQ ID NO: 29 - DNA, 20 bp, Artificial Sequence
  primer
gcgcagcctattgtccaata

SEQ ID NO: 30 - DNA, 26 bp, Artificial Sequence
  primer
aggaggtggcacagctaaataacgcg

SEQ ID NO: 31 - DNA, 30 bp, Artificial Sequence
  primer
cacctgacactgcacaacaggttaaaaagc

SEQ ID NO: 32 - DNA, 30 bp, Artificial Sequence
  primer
caccatggataaagcggaattaattcccga

SEQ ID NO: 33 - DNA, 30 bp, Artificial Sequence
  primer
caccatgaagctactgtcttctatcgaaca

SEQ ID NO: 34 - DNA, 25 bp, Artificial Sequence
  primer
cctatcaactttcgatggtaggata

SEQ ID NO: 35 - DNA, 27 bp, Artificial Sequence
  primer
cgttaagggatttagattgtactcatt

SEQ ID NO: 36 - DNA, 25 bp, Artificial Sequence
  primer
gtaggcaggctctctagcctctagg

SEQ ID NO: 37 - DNA, 23 bp, Artificial Sequence
  primer
aacctgacattatatattgcacc

SEQ ID NO: 38 - DNA, 26 bp, Artificial Sequence
  primer
ctcagggttcctgccggtagaaagca

SEQ ID NO: 39 - DNA, 19 bp, Artificial Sequence
  primer
cgaaacgaacagtagctag

SEQ ID NO: 40 - DNA, 20 bp, Artificial Sequence
  primer
agagagggaagcctgaggag

SEQ ID NO: 41 - DNA, 20 bp, Artificial Sequence
  primer
cttgagcttgctctccacct

SEQ ID NO: 42 - DNA, 22 bp, Artificial Sequence
  primer
cttgacgccgaggagctcgaat

SEQ ID NO: 43 - DNA, 20 bp, Artificial Sequence
  primer
gcctgcattttggagatcgg

SEQ ID NO: 44 - DNA, 20 bp, Artificial Sequence
  primer
ttatgccgttgtctgcttcc

SEQ ID NO: 45 - DNA, 20 bp, Artificial Sequence
  primer
ctactggaggattatggtca

SEQ ID NO: 46 - DNA, 25 bp, Artificial Sequence
  primer
ccatggacggacatgagcagcattt

SEQ ID NO: 47 - DNA, 20 bp, Artificial Sequence
  primer
aagatgatcagggacgatga

SEQ ID NO: 48 - DNA, 20 bp, Artificial Sequence
  primer
catcgcgcaacccgaacatg

SEQ ID NO: 49 - DNA, 21 bp, Artificial Sequence
  primer
tgtcgatcaggctcctcagag

SEQ ID NO: 50 - DNA, 20 bp, Artificial Sequence
  primer
tgagccagctctcatcctgc

SEQ ID NO: 51 - DNA, 20 bp, Artificial Sequence
  primer
gagccgatagaaactgaggg

SEQ ID NO: 52 - DNA, 20 bp, Artificial Sequence
  primer
tgcagattacgctaattcat

SEQ ID NO: 53 - DNA, 21 bp, Artificial Sequence
  primer
cctcttatagctaactttagc

SEQ ID NO: 54 - DNA, 20 bp, Artificial Sequence
  primer
cgcctacgagcctggatcaa

SEQ ID NO: 55 - DNA, 20 bp, Artificial Sequence
  primer
taaacacaaggcaattaaca

SEQ ID NO: 56 - DNA, 20 bp, Artificial Sequence
  primer
aaacggctagctcgaacaat

SEQ ID NO: 57 - DNA, 20 bp, Artificial Sequence
  primer
ctaatcgcaggctcaatcac

SEQ ID NO: 58 - DNA, 307 bp, Oryza sativa
gctattagtacaaaaaaaataataatttttacagttagagcaaaaagccattgatctccttttggctggtagagttgttactgctacaactgcttactattagtaactatataattataattataattgcaatgcataaggtccaagtttgttgtgatctactatgattctagtaactctctggtttttctgagtcctgacctgattaagaagacatgtatcaactatgtatatctatgaactgacctaacttgaggctatcattaactaatgatggtttatgattagtcaattgctttgcttttga

SEQ ID NO: 59 - DNA, 245 bp, Oryza sativa
ctcaagaaaaaaaaatctaggtttctgcttcttctcttgtctgaaaattttaggggtgtgagagaaatcatcagtgttgttgttactgctgctgctgctgctatatgatcaagatatatataacaaaaaaaaagaactccatttgtttgtgtgcttgtctctggatgaactctgatcttgatgatgatgatgaatcttgtctgtctggcatgaggtcaacaactcaacattgctatgaacaaaaa

SEQ ID NO: 60 - DNA, 750 bp, Artificial Sequence
  DNA sequence for construct
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacgggagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtctagataggagatccgtcgacctgcagatcgt

(19) United States
(12) Patent Application Publication
(10) Pub. No.: US 2015/0284735 A1
(43) Pub. Date: Oct. 8, 2015

(54) SKIN GENE SILENCING PLASMID, AND TRANSFORMED PLANT CELL AND TRANSGENIC PLANT COMPRISING THE SAME

(71) Applicant: Academia Sinica, Taipei (TW)

(72) Inventors: SU-MAY YU, Taipei (TW); CHIEN-RU LIN, Taipei (TW)

(21) Appl. No.: 14/606,159

(22) Filed: Jan. 27, 2015

Related U.S. Application Data
(60) Provisional application No. 61/932,426, filed on Jan. 28, 2014.

(51) Int. Cl. C12N 15/82

(52) U.S. Cl. CPC C12N 15/8245

(57) ABSTRACT

The present invention provides relates to a SKIN gene silencing plasmid comprising a promoter; two DNA fragments, which are obtained from one DNA fragment derived from the cDNA of SKIN1 or SKIN2 and arranged in sense and antisense orientation; and a third DNA fragment inserted between the two DNA fragments. SKIN is a negative regulator of the growth and yield of rice, and decreasing the expression of endogenous SKIN increases the yield of rice. The present invention also relates to a transformed plant cell and a transgenic plant comprising the SKIN gene silencing plasmid.

### BACKGROUND OF THE INVENTION

The plant life cycle is accompanied by source-sink transitions that modulate nutrient assimilation and partitioning during growth and development. The regulation of source-sink communication determines the pattern of carbon allocation in whole plant and plays a pivotal role in determining crop productivity. Most studies have been focused on the carbon supply and demand process that regulates the expression of genes involved in carbohydrate production and reserve mobilization in source tissues (photosynthetic leaves and storage organs) and utilization in sink tissues (growing vegetative and reproductive tissues). However, components in underlying signal transduction pathways that regulate source-sink communication are largely unknown. Insight into the regulatory mechanisms is not only significant for understanding how sugar starvation/demand regulates plant growth and development, but also important for genetic manipulation of source-sink nutrient allocation for crop improvement.

The source-sink transition during germination and seedling growth in cereals can be viewed within a nutrient supply-demand paradigm, and represents an ideal system to study the mechanism of nutrient demand/starvation signaling and gene regulation in source-sink communication. Germination followed by seedling growth constitutes two essential steps in the initiation of the new life cycle in plants, and completion of these steps requires coordinated developmental and biochemical processes, including mobilization of reserves in seeds (the source tissue) and elongation of the embryonic axis (the sink tissue). In these processes in cereals, the stored reserves in the endosperm are degraded and mobilized by a battery of hydrolases to sugars and other nutrients that are absorbed by the scutellum and transported to the embryonic axis to support seedling growth (Akazawa and Hara-Nishimura, 1985; Beck and Ziegler, 1989; Fincher, 1989; Woodger et al., 2004). Starch, which constitutes approximately 75% of cereal grain dry weight (Kennedy, 1980), provides the major carbon source for generating energy and metabolites during germination and seedling growth. Consequently, among all hydrolases, α-amylases are the most abundant and play a central role in the mobilization of starch and thus the rate of seedling growth. The expression of α-amylase is induced by both the hormone gibberellin (GA) and sugar demand/starvation (Yu, 1999a; Yu, 1999b; Lu et al., 2002; Sun and Gubler, 2004; Woodger et al., 2004; Chen et al., 2006; Lu et al., 2007; Lee et al., 2009), which has served as a model for studying the mechanism of sugar starvation signaling and crosstalk with the GA signaling pathway.

Our previous studies in rice revealed that sugar starvation regulates α-amylase expression by controlling its transcription rate and mRNA stability (Sheu et al., 1994; Sheu et al., 1996; Chan and Yu, 1998). Transcriptional regulation is mediated through a sugar response complex (SRC) in α-amylase gene promoters, in which the TA box is a key regulatory element (Lu et al., 1998; Chen et al., 2002; Chen et al., 2006). MYBS1 is a sugar repressible R1 MYB transcription factor that interacts with the TA box and induces α-amylase gene promoter activity in rice suspension cells and germinating embryos under sugar starvation (Lu et al., 2002; Lu et al., 2007). GA also activates α-amylase gene promoters through the GA response complex (GARC) in which the adjacent GA response element (GARE) and the TA/Amy box are key elements and act synergistically (Rogers et al., 1994; Gubler et al., 1999; Gomez-Cadenas et al., 2001). MYBGA (also called GAMYB) is a GA-inducible R2R3 MYB that binds to the GARE and activates promoters of α-amylases and other hydrolases in cereal aleurone cells in response to GA (Gubler et al., 1995; Gubler et al., 1999; Hong et al., 2012). Our recent study revealed that the nuclear import of MYBS1 is repressed by sugars, and GA antagonizes sugar repression by enhancing the co-nuclear transport of MYBGA and MYBS1 and formation of a stable bipartite MYB-DNA complex to activate α-amylase gene promoters (Hong et al., 2012). Furthermore, not only sugar but also nitrogen and phosphate starvation signals converge and crosstalk with GA to promote the co-nuclear import of MYBS1 and MYBGA and expression of hundreds of GA-inducible but functionally distinct hydrolases, transporters and regulators for mobilization of the full complement of nutrients to support active seedling growth (Hong et al., 2012).

The rice Snf1-related protein kinase 1 (SnRK1) family, SnRK1A and SnRK1B, are structurally and functionally analogous to their yeast and mammalian counterparts, the sucrose non-fermenting 1 (SNF1) and AMP-activated protein kinase (AMPK), respectively (Lu et al., 2007). SNF1, AMPK and SnRK1 are Ser/Thr protein kinases and considered as fuel gauge sensors monitoring cellular carbohydrate status and/or AMP/ATP levels in order to maintain equilibrium of sugar production and consumption necessary for proper growth (Halford et al., 2003; Hardie and Sakamoto, 2006; Rolland et al., 2006; Polge and Thomas, 2007). SNF1, AMPK and SnRK1 are heterotrimeric protein complexes, consisting of a catalytic activating subunit (α or Snf1) and two regulatory subunits (β and γ or Sip1/Sip2/Ga183 and Snf4) (Polge and Thomas, 2007). These protein kinases can be divided into N-terminal kinase domain (KD) and C-terminal regulatory domain (RD) (Dyck et al., 1996; Jiang and Carlson, 1996, 1997; Crute et al., 1998; Lu et al., 2007). In glucose-replete yeast cells, the SNF1 complex exists in an inactive autoinhibited conformation in which the Snf1 KD binds to the Snf1 RD (Jiang and Carlson, 1996). In glucose-starved yeast cells, Snf4 binds to the Snf1 RD and the Snf1 KD is released, leading to an active open conformation Snf1 (Jiang and Carlson, 1996). Sip1/Sip2/Ga183 acts as a scaffold protein binding to both Snf1 and Snf4, and this binding is also promoted by glucose starvation (Jiang and Carlson, 1996, 1997).

The conserved inter- and intra-subunit interactions and functions of SnRK1 protein kinases have also been demonstrated in the sugar starvation signaling pathway in rice, and SnRK1A acts upstream and plays a central role in the sugar starvation signaling pathway activating MYBS1 and α-amylase expression in rice (Lu et al., 2007). Recently, we found that CIPK15 [Calcineurin B-like (CBL)-interacting protein kinase 15] acts upstream of SnRK1A and plays a key role in O₂ deficiency tolerance in rice (Lee et al., 2009). CIPK15 regulates the accumulation of SnRK1A protein, as well as interacts with SnRK1A, and links O₂ deficiency signals to the SnRK1A-dependent sugar starvation sensing cascade to regulate sugar and energy production and to program rice growth under flood conditions (Lee et al., 2009).

In plants, SnRK1s have been proposed to coordinate and adjust physiological and metabolic demands for growth, including regulation of carbohydrate metabolism, starch biosynthesis, fertility, organogenesis, senescence, stress responses, and interactions with pathogens (Polge and Thomas, 2007). SnRK1 regulates carbohydrate metabolism and development in crop sinks such as potato tubers (McKibbin et al., 2006) and legume seeds (Radchuk et al., 2010). SnRK1 overexpression increases starch accumulation in potato tubers (Purcell et al., 1998; Halford et al., 2003), and SnRK1 silencing causes abnormal pollen development and male sterility in transgenic barley (Zhang et al., 2001). SnRK1 (KIN10/11) activates genes involved in degradation processes and photosynthesis and inhibits those involved in biosynthetic processes in Arabidopsis (Baena-Gonzalez et al., 2007).

However, the mechanism regulating the source-sink communication during plant growth and development is not clearly understood. Thus there is need to study genes involved in sugar and nutrient demand signaling between source and sink tissues.

### SUMMARY OF THE INVENTION

The present invention provides a novel abiotic stress-inducible plant specific gene family, SKIN1 and SKIN2, which interact with and repress the function of SnRK1A. We found that sugar demand signals from the sink tissue (germinated embryo) were transmitted via SnRK1A to induce the expression of a full complement of enzymes necessary for the production of sugar and other nutrients in the source tissue (starchy endosperm). By using abscisic acid (ABA), a plant hormone, as a stress signaling inducer, we further discovered that SKINs repress the SnRK1A-dependent sugar/nutrient starvation signaling by inhibiting the co-nuclear import of SnRK1A and MYBS1 and thus inhibit their functions in inducing enzyme expression facilitating nutrient mobilization under abiotic stress conditions.

The present invention provides a SKIN gene silencing plasmid, comprising a promoter; two DNA fragments, which are obtained from one DNA fragment derived from the cDNA of SKIN1 or SKIN2 and arranged in sense and antisense orientation; and a third DNA fragment inserted between the two DNA fragments. Preferably, the third DNA sequence is derived from the cDNA of GFP. More preferably, the one DNA fragment derived from the cDNA of SKIN1 is SEQ ID No: 58 (307 bp), the one DNA fragment derived from the cDNA of SKIN2 is SEQ ID No: 59 (245 bp); and the third DNA sequence is SEQ ID No: 60 (750 bp).

In one preferred embodiment of the SKIN gene silencing plasmid, the promoter is selected from 35CaMV, actin1, GluB1, rbcS, cab, SNAC1, pin2, SAG12, Psam1, TobRB7 or ubiquitin promoter.

The present invention also provides a transformed plant cell, which comprises the above-mentioned SKIN gene silencing plasmid. Specifically, the SKIN gene silencing plasmid comprises a promoter; two DNA fragments, which are obtained from one DNA fragment derived from the cDNA of SKIN1 or SKIN2 and arranged in sense and antisense orientation; and a third DNA fragment inserted between the two DNA fragments. Preferably, the third DNA sequence is derived from the cDNA of GFP. More preferably, the one DNA fragment derived from the cDNA of SKIN1 is SEQ ID No: 58 (307 bp), the one DNA fragment derived from the cDNA of SKIN2 is SEQ ID No: 59 (245 bp); and the third DNA sequence is SEQ ID No: 60 (750 bp).

In one preferred embodiment of the transformed plant cell, the promoter is selected from 35CaMV, actin1, GluB1, rbcS, cab, SNAC1, pin2, SAG12, Psam1, TobRB7 or ubiquitin promoter.

In one preferred embodiment of the transformed plant cell, the plant is a monocot selected from maize, wheat, barley, millet, sugarcane, Miscanthus, switchgrass or sorghum.

In one preferred embodiment of the transformed plant cell, the plant is a dicot selected from Arabidopsis, tomato, potato, brassica, soybean, canola or sugarbeet.

The present invention also provides a transgenic plant, which comprises the above-mentioned SKIN gene silencing plasmid. Specifically, the SKIN gene silencing plasmid comprises a promoter; two DNA fragments, which are obtained from one DNA fragment derived from the cDNA of SKIN1 or SKIN2 and arranged in sense and antisense orientation; and a third DNA fragment inserted between the two DNA fragments. Preferably, the third DNA sequence is derived from the cDNA of GFP. More preferably, the one DNA fragment derived from the cDNA of SKIN1 is SEQ ID No: 58 (307 bp), the one DNA fragment derived from the cDNA of SKIN2 is SEQ ID No: 59 (245 bp); and the third DNA sequence is SEQ ID No: 60 (750 bp).

In one preferred embodiment of transgenic plant, the promoter is selected from 35CaMV, actin1, GluB1, rbcS, cab, SNAC1, pin2, SAG12, Psam1, TobRB7 or ubiquitin promoter.

In one preferred embodiment of transgenic plant, the plant is a monocot selected from maize, wheat, barley, millet, sugarcane, Miscanthus, switchgrass or sorghum.

In one preferred embodiment of transgenic plant, the plant is a dicot selected from Arabidopsis, tomato, potato, brassica, soybean, canola or sugarbeet.

### BRIEF DESCRIPTION OF THE DRAWINGS

FIG. 1. A novel family of GKSKSF domain (KSD, SEQ ID No: 61)-containing regulatory proteins. (A) Sequence comparison among KSD-containing proteins in plants, including OsSKIN2 (SEQ ID No: 4), ZmKCP (SEQ ID No: 62), OsSKIN1 (SEQ ID No: 2), ZmMTD1 (SEQ ID No: 63), Sorghum02g028960 (SEQ ID No: 64), Zm-MTD186T7R4 (SEQ ID No: 65), AtKCL1 (SEQ ID No: 66), AtKCL2 (SEQ ID No: 67), AtKCP (SEQ ID No: 68), BnKCP1 (SEQ ID No: 69). Identical amino acids are shown as white letters on a black background and similar amino acids are indicated as black letter on a gray background. Boxes indicate GKSKSF domain (KSD), putative nuclear localization signal (NLS) and protein kinase A-inducible domain (KID). Asterisks denote conserved domains in monocots. (B) Phylogenic analysis of KSD-containing proteins in plants. The scale value of 0.1 indicates 0.1 amino acid substitutions per site. The colored area denotes the monocot specific gene cluster.

FIG. 2. The N-terminus of SKIN interacts with the kinase domain of SnRK1A. For the GAL4-UAS two-hybrid assay, rice embryos were co-transfected with effector and reporter plasmids, incubated in −S medium for 24 h, and assayed for luciferase activity. The luciferase activity in rice embryos bombarded with effectors Ubi:GAD, Ubi:GBD and reporter 5XUAS-35S mp:Luc was set to 1×, and other values were calculated relative to this value. Error bars indicate the SE for three replicate experiments. Significance levels: * p<0.1, ** p<0.05. The Y axis indicates the relative luciferase activity with different constructs. (A) Plasmid constructs. (B) Rice embryos were co-transfected with effectors Ubi:GAD-SnRK1A and Ubi:GBD-SKIN (wild type or truncated) and reporter 5XUAS-35S mp:Luc. (C) Rice embryos were co-transfected with effectors Ubi:GAD-SnRK1A [wild type, kinase domain (KD) or regulatory (RD)], Ubi:GBD-SKIN and reporter 5XUAS-35S mp:Luc. (D) Rice embryos were co-transfected with effectors Ubi:GAD-SnRK1A and Ubi:GBD-SKIN and reporter 5XUAS-35S mp:Luc, and incubated in −S medium containing 1 μM ABA.

FIG. 3. The highly conserved GKSKSF domain (KSD) is essential for SKINs to antagonize the function of SnRK1A. Rice embryos were transfected with plasmids, incubated in medium with 100 mM glucose (+S) or without glucose (−S) for 24 h, and assayed for luciferase activity. The luciferase activity in rice embryos bombarded with the SRC-35S mp-Luc construct only and in +S medium was set to 1×, and other values were calculated relative to this value. Error bars indicate the SE for three replicate experiments. (A) Plasmid constructs. (B) Rice embryos were co-transfected with effector Ubi:SnRK1A, Ubi:SKIN1 or Ubi:SKIN(Ri) alone and reporter SRC-35Smp:Luc, or co-transfected with effectors Ubi:SnRK1A and Ubi:SKINor Ubi:SKIN(Ri) and reporter SRC-35Smp:Luc. (C) Total cellular proteins were extracted from rice embryos transfected with Ubi:SnRK1A, Ubi:SKIN or Ubi:SnRK1A and Ubi:SKIN by particle bombardment and subjected to Western blot analysis using antibodies against SnRK1A and the HA tag fused to at the N-terminus of SKINs. Protein loading control by the Ponceau S staining is shown in FIG. 15A. (D) Rice embryos were co-transfected with effectors Ubi:SnRK1A and Ubi:SKIN1 (wild-type or truncated) and reporter SRC-35Smp:Luc. (E) Rice embryos were co-transfected with effectors Ubi:SnRK1A and Ubi:SKIN (wild-type, KSD deleted, or KSD replaced with 6 Ala) and reporter SRC-35Smp:Luc.

FIG. 4. SKIN suppresses the SnRK1A-dependent sugar and nutrient starvation signaling pathway. (A) Two-day-old seedlings from the wild type and transgenic lines SKIN1-Ox (O3), SKIN1-Ri (R3), SKIN2-Ox (O2), SKIN2-Ri (R1) were grown under +S or −S condition with 14 h light/10 h dark cycle for 18 h. Total RNA was purified from cells and subjected to quantitative RT-PCR analysis using primers specific for indicated genes, and mRNA levels were normalized against the level of Act1 mRNA. The lowest mRNA level of wild-type was set to 1× and other samples were calculated relative to this value. The highest mRNA level was set as 100%. Error bars indicate the SE for three replicate experiments. (B) Total proteins were extracted from two-day-old seedlings of SKIN-Ox transgenic lines and subjected to Western blot analysis using antibodies against SnRK1A and the HA tag fused to the N-terminus of SKINs. Protein loading control by the Ponceau S staining is shown in FIG. 15B.

FIG. 5. SKINs repress seedling growth by inhibiting nutrient mobilization in the endosperm. Transgenic lines SKIN1-Ox(O3), SKIN1-Ri(R3), SKIN2-Ox(O2) and SKIN2-Ri(R1) were used in the following experiments. (A) Seeds were germinated and grown in water at 28° C. under a 14-h light/10-h dark cycle or continuous darkness without (panel 1) or with (panel 2) 3% (90 mM) sucrose for 6 days. (B) Lengths of shoots and roots of seedlings in (A) were quantified. Panels 1 and 2, without and with sucrose, respectively. (C) Seedlings were grown under a 14-h light/10-h dark cycle or continuous darkness for 3 days. Total RNA was extracted and subjected to quantitative (real-time) RT-PCR analysis using primers specific for αAmy3 (panel 1) and EP3A (panel 2). Error bars represent SE (n=12) at significance levels: *p<0.1, **p<0.05 in (B) and (C).

FIG. 6. SKINs suppress sugar production necessary for seedling growth under hypoxia. Rice seeds were germinated in air or in water with or without 90 mM sucrose at 28° C. under a 14-h light/10-h dark cycle for various lengths of time. Shoot length of seedlings were measured daily. Error bars indicate the S.E. of shoot length (n=10). Panel 1: transgenic line SKIN1-04 overexpressing SKIN1; panel 2: transgenic line SKIN2-04 overexpressing SKIN2. For data using more SKIN-Ox and SKIN-Ri lines, see also FIG. 16 online.

FIG. 7. SKIN and SnRK1A interact primarily in the cytoplasm. Barley aleurones were transfected with plasmid constructs and incubated in −S medium for 24 h. Thirty optical sections of 0.9-1.1 μm thickness were prepared for each cell and only five regularly spaced sections (sections 3, 9, 15, 21 and 27) are shown here. C and N indicate higher GFP signals and c and n indicate lower GFP signals in the cytoplasm and nucleus, respectively. For more section images of each cell, see also FIG. 17 online.

FIG. 8. SKINs could antagonize the function of SnRK1A in both the cytoplasm and nucleus. (A) Plasmid constructs. (B) Barley aleurone cells were bombarded with Ubi:SKIN-GFP or Ubi:SKINΔNLS-GFP. Cells were incubated in +S or −S medium for 24 h. Thirty optical sections of 0.9-1.1 μm thickness were prepared for each cell and only five regularly spaced sections (sections 3, 9, 15, 21 and 27) are shown here. C and N indicate higher GFP signals and c and n indicate lower GFP signals in the cytoplasm and nucleus, respectively. For more section images of teach cell, see also FIG. 18 online. (C) Rice embryos were co-transfected with SnRK1A and Ubi:SKIN-GFP or Ubi:SKINΔNLS-GFP and incubated in +S or −S medium for 24 h, and assayed for luciferase activity. The luciferase activity in rice embryos bombarded with the SRC-35S mp-Luc construct only and in +S medium was set to 1×, and other values were calculated relative to this value. Error bars indicate the SE for three replicate experiments.

FIG. 9. The expression of SKIN is induced by various abiotic stresses and ABA, and SKINs promote the ABA sensitivity. (A) Total RNA was purified from leaves of 2-week-old rice seedlings that had been air dried, treated with 200 mM salt, incubated at 4° C., or treated with 1 μM ABA, or from embryos of seedlings grown underwater (hypoxia), for various lengths of time. RNAs were subjected to quantitative RT-PCR analysis using primers specific for SKIN1 and SKIN2. The highest mRNA level was set as 100%. The lowest mRNA level was assigned a value of 1× and mRNA levels of other samples were calculated relative to this value. Error bars indicate the SE for three replicate experiments. (B) Seeds of transgenic lines SKIN1-Ox(O3), SKIN1-Ri(R3), SKIN2-Ox(O2) and SKIN2-Ri(R1) were germinated and grown in water containing various concentrations of ABA at 28° C. under a 14-h light/10-h dark cycle for 6 days. Lengths of shoots were measured. Error bars represent SE (n=8) at significance levels: *p<0.1, ** p<0.05. For photos of treated seedlings, see also FIG. 20 online.

FIG. 10. ABA restricts SKINs, SnRK1A and MYBS1 in the cytoplasm under sugar starvation. Barley aleurones were co-transfected with indicated plasmid constructs and incubated in +S or −S medium with ABA (+ABA) or without ABA (−ABA) for 48 h. Thirty optical sections of 0.9-1.1 μm thickness were prepared for each cell and only five regularly spaced sections (sections 3, 9, 15, 21 and 27) are shown here. C and N indicate higher GFP signals, and c and n indicate lower GFP signals in the cytoplasm and nucleus, respectively. For more section images of each cell, see also FIG. 22 online. (A) Barley aleurones were transfected with Ubi:SKIN1-GFP, Ubi:SKIN2-GFP, Ubi:SnRK1A-GFP or Ubi:MYBS1-GFP alone. (B) Barley aleurones were transfected with Ubi:MYBS1-GFP alone (panel 1) or co-transfected with Ubi:MYBS1-GFP and Ubi:SnRK1A (panel 2), or with Ubi:MYBS1-GFP and Ubi:SnRK1A(Ri) (panel 3). (C) Barley aleurones were co-transfected with Ubi:SnRK1A-GFP and Ubi:SKIN(Ri). (D) Wild type rice (WT) or transgenic rice overexpression Ubi:SKIN(Ri) were transfected with Ubi:SnRK1A-GFP (panels 1-3) or Ubi:MYBS1-GFP (panels 4-6).

FIG. 11. SnRK1A plays a central role regulating the source-sink communication for nutrient mobilization in cereal seedlings, and differential cellular localization of key factors regulates the process under abiotic stress. Sugar starvation signals from sink tissues (germinating embryo and seedling) in demand of nutrients trigger the co-nuclear localization of SnRK1A and MYBS1, leading to the induction of hydrolases necessary for the mobilization of nutrients in the source tissue (endosperm). Stress and ABA facilitate the cytoplasmic localization of SKIN which binds to SnRK1A and prevents SnRK1A and MYBS1 from entering the nucleus. More details are described in the text.

FIG. 12. SKIN1 and SKIN2 interact with SnRK1A in yeast. In the yeast 2-hybrid assay, plasmid constructs ADH1:GAD-SnRK1A and ADH1:GBD-SKIN were used as effectors, and Mel1:LacZ, Mel1:Mel1 and Gal1:HIS3 as reporters. Yeast strain AH109 containing GADSnRK1A or GAD alone (—) was mated with yeast strain Y187 containing GBD-SKIN or GBD alone (—). The interaction between p53 and large T-antigen (T-Ag) was used as a positive control.

FIG. 13. Amino acid sequence alignment between SKIN1 (SEQ ID No: 2) and SKIN2 (SEQ ID No: 4). Identical amino acids are shown as white letters on a black background and similar amino acids are indicated as black letter on a gray background. Abbreviation of functional domains: NLS, nuclear localization signal; KSD, GKSKSF domain. KID, protein kinase A inducible domain.

FIG. 14. The N-terminal amino acids 1-83 of SKIN1 interact with the kinase and autoinhibitory domains of SnRK1A in yeast. (A) Plasmid constructs ADH1:GAD-SnRK1A and ADH1:GBD-SKIN1 (wild type or deletion at N- or C-terminus) were used as effectors, and Mel1:LacZ, Mel1:Mel1 and Gal1:HIS3 were used as reporters. (B) The N-terminus of SKIN1 interacts with SnRK1A in the yeast two-hybrid assays. (C) The kinase domain (KD) and auto-inhibitory domain (AID) of SnRK1A interact with SKIN1 and SKIN2. Yeast strain AH109 containing GAD-SnRK1A or GAD alone (—) was mated with yeast strain Y187 containing various GBD-SKIN1 constructs or GBD alone (—). The interaction between p53 and large T-antigen (T-Ag) was used as a positive control.

FIG. 15. Ponceau S staining of nitrocellulolose membrane to visualize the protein loading in Western blot analysis. (A) Rice embryos transfected with Ubi.SnRK1A, Ubi.SKIN or Ubi.SnRK1A and Ubi.SKIN by particle bombardment. Total proteins were extracted and blotted to the nitrocellulose membrane for Western blot analysis shown in FIG. 3C. The same nitrocellulose membrane was then stained with Ponceau S. Proteins in lanes 1-8 were electrophoresed in one gel and lanes 9-12 in another gel. NT: non-transfected embryos. (B) Total proteins were extracted from two-day-old seedlings of SKIN-Ox transgenic lines and blotted to the nitrocellulose membrane for Western blot analysis shown in FIG. 4B. The same nitrocellulose membrane was then stained with Ponceau S. Proteins in lanes 1-4 were electrophoresed in one gel and lanes 5-8 in another gel. WT: wild type seedlings

FIG. 16. SKINs suppress sugar production necessary for underwater seedling growth. Rice seeds of SKIN-Ox and SKIN-Ri lines were germinated in air or in water with or without 90 mM sucrose for various lengths of time. Shoot length of seedlings were measured daily. Error bars indicate the S.E. of shoot length (n=10). Panel 1: in air; panel 2: in water; panel 3: in water with sucrose. Data for representative lines are also shown in FIG. 6.

FIG. 17. SKIN and SnRK1A interact primarily in the cytoplasm. Barley aleurones were transfected with plasmid constructs and incubated in −S medium for 24 h. Thirty optical sections of 0.9-1.1 μm thickness were prepared for each. C and N indicate higher GFP signals and c and n indicate lower GFP signals in the cytoplasm and nucleus, respectively. Boxes indicate images shown in FIG. 7.

FIG. 18. SKINs without NLSs are localized in the cytoplasm. Barley aleurone cells were bombarded with Ubi:SKINΔNLS-GFP). Cells were treated with 100 mM glucose (+S) or without glucose (−S) for 24 h. Thirty optical sections of 0.9-1.1 μm thickness were prepared for each cell. C and N indicate higher GFP signals and c and n indicate lower GFP signals in the cytoplasm and nucleus, respectively. Boxes indicate images shown in FIG. 7B.

FIG. 19. SKIN is expressed in most rice tissues. Total RNA was purified from rice seedlings (7-day-old), mature plants (3-month-old), flowers and immature panicles (1-22 days after pollination, DAF). RNAs were subjected to quantitative RT-PCR analysis using primers specific for SKIN1 and SKIN2. The highest mRNA level was set as 100%. The lowest mRNA level was assigned a value of 1× and mRNA levels of other samples were calculated relative to this value. Error bars indicate the SE for three replicate experiments.

FIG. 20. Growth of transgenic rice overexpressing SKIN is more sensitive to ABA inhibition. Seeds of transgenic lines SKIN1-Ox(O3), SKIN1-Ri(R3), SKIN2-Ox(O2) and SKIN2-Ri(R1) were germinated and grown in water containing various concentrations of ABA for 6 days at 28 C under a 14-h light/10-h dark cycle. Seedlings were photographed at day 6. The quantitative data of shoot length are shown in FIG. 9B.

FIG. 21. ABA and sorbitol suppress the function of SnRK1A in activation of αAmy3 SRC promoter. Rice embryos and barley aleurones were transfected with reporter SRC-35Smp:Luc with or without effectors, incubated with or without ABA for 24 h, and assayed for luciferase activity. The luciferase activity in embryos or aleurones bombarded with the SRC-35S mp-Luc construct only and in +S medium was set to 1×, and other values were calculated relative to this value. Error bars indicate the SE for three replicate experiments. (A) Plasmid constructs. (B) Barley aleurones were transfected with effector Ubi:SnRK1A, Ubi:SKIN1 or Ubi:SKIN(Ri) alone and reporter SRC-35Smp:Luc, or co-transfected with effectors Ubi:SnRK1A and Ubi:SKIN or Ubi:SKIN(Ri) and reporter SRC-35Smp:Luc. (C) Rice embryos (upper panel) were transfected with or without Ubi:SnRK1A and incubated with or without 1 μM ABA or 50 mM sorbitol, and barley aleurones (lower panel) were transfected with or without Ubi:SnRK1A and incubated with 5 μM ABA or 400 mM sorbitol.

FIG. 22. ABA restricts SKINs, SnRK1A and MYBS1 in the cytoplasm under sugar starvation. Barley aleurones were co-transfected with indicated plasmid constructs and incubated in +S or −S medium with ABA (+ABA) or without ABA (−ABA) for 48 h. Thirty optical sections of 0.9-1.1 μm thickness were prepared for each cell and only five regulatory spaced sections (sections 3, 9, 15, 21 and 27) are shown here. C and N indicate higher GFP signals and c and n indicate lower GFP signals in the cytoplasm and nucleus, respectively. Boxes indicate images shown in FIG. 10. (A) Barley aleurones were transfected with SKIN1-GFP, SKIN2-GFP, SnRK1A-GFP or MYBS1-GFP alone. (B) Barley aleurones were transfected with MYBS1-GFP alone or co-transfected with MYBS1-GFP and SnRK1A or SnRK1A(Ri). (C) Barley aleurones were co-transfected with SnRK1A-GFP and SKIN(Ri). (D) Wild type rice (WT) or transgenic rice overexpressing SKIN(Ri) were transfected with SnRK1A-GFP or MYBS1-GFP.

FIG. 23. SKIN1 but not SKIN2 hampers seed development by repression of enzymes essential for starch and GA biosynthesis. (A) Same numbers of seeds of transgenic lines SnRK1A-Ri (127-13), SKIN1-Ox (O3) and SKIN1-Ri (R3) were lined up head-to-tail for length comparison (upper panel) and side-by-side for width comparison (lower panel). (B) The 1000-grain weight (upper panel), grain length, thickness and width and grain yield per plant (lower panel) of three independent transgenic plants each of SKIN1-Ox, SKIN1-Ri and SnRK1A-Ri lines were determined. (C) Total RNA was extracted from immature panicles of transgenic lines SKIN1-Ox(O3), SKIN1-Ri(R3), SKIN2-Ox(O2) and SKIN2-Ri(R1) and subjected to quantitative RT-PCR analysis using primers specific for SKIN1, SKIN2, GIF and GA3ox2. The highest mRNA level was set as 100%. The lowest mRNA level of wild type was assigned a value of 1× and other samples were calculated relative to this value. Error bars indicate the SE (n=12) for three replicate experiments. Significance levels: *p<0.1, * * p<0.05

FIG. 24. SKIN retarded plant growth. Wild type and transgenic lines SKIN1-Ox(O3), SKIN1-Ri(R3), SKIN2-Ox(O2) and SKIN2-Ri(R1) were used in the experiment. Plant heights at heading stage were measured, with ±indicating SE (n=9).

FIG. 25 Wild type (WT), three independent SKIN1-Ox lines (O2, O3 and O6) and three independent SKIN1-Ri lines (R2, R3 and R5) were grown in non-irrigated fields in the spring (February to July) of 2014. Grain yield was determined after harvest. Error bars represent SD (n=10). Significance levels with the t-test: * P<0.05, ** P<0.01, *** P<0.001.

### DETAILED DESCRIPTION OF THE INVENTION

### Materials and Methods

### Plant Materials

Rice (Oryza sativa cv Tainung 67) and barley (Hordeum vulgare cv Himalaya) were used in this study. Embryo calli were induced in the Murashige & Skoog (MS) medium containing 3% sucrose and 10 mM 2,4-D (2,4-Dichlorophenoxyacetic acid) for 5 days. For hydroponic culture of rice seedlings, seeds were sterilized with 1.5% NaOCl plus Tween 20 for 1 h, washed extensively with distilled water, and germinated in a petri dish with wetted filter papers at 28° C. under a 14-h light/10-h dark condition unless otherwise indicated. The SnRK1A Knockdown transgenic rice was generated previously (Lu et al., 2007).

Our previous studies showed that sugar regulations of MYBS1 function in barley aleurones (Lu et al., 2002), SnRK1A regulation of MYBS1 function using rice embryos (Lu et al., 2007), CIPK15 regulation of SnRK1A expression using rice suspension cells (Lee et al., 2009), and regulation of MYBS1 and MYBGA interaction and nucleocytoplasmic shuttling of MYBS1 using rice and barley aleurones (Hong et al., 2012) are all consistent regardless of different systems being used. For transient expression assay of luciferase activity, aleureones/embryos are preferred as compared with rice endosperms due to easier manipulation for large-scale sample preparation, particle bombardment and protein extraction. For cellular localization of GFP fused to target protein, barley aleurones are preferred as the rice aleurone has a single layer of cells and is fragile, while the barley aleurone has 3-4 layers and is relatively stronger and easier to manipulate under the microscope. Additionally, barley or rice aleurone cells have relatively much larger nuclei but smaller vacuoles as compared with onion epidermal cells, which facilitate the study on nuclear import of proteins.

### Plasmids

Plasmid p3Luc.18 contains αAmy3 SRC (−186 to −82 upstream of the transcription start site) fused to the CaMV35S minimal promoter-Adh1 intron-luciferase cDNA (Luc) fusion gene (Lu et al., 1998). Plasmid pUG contains β-glucuronidase cDNA (GUS) fused between the Ubi promoter and Nos terminator (Christensen and Quail, 1996). Plasmid pUbi-SnRK1A-Nos contains SnRK1A full-length cDNA between a Ubi promoter and a Nos terminator (Lu et al., 2007). Plasmid pUbi-SnRK1A-KD-Nos contains a cDNA encoding the kinase domain of SnRK1A between the Ubi promoter and Nos terminator (Lu et al., 2007). Plasmid pUbi-SnRK1A-RD-Nos contains a cDNA encoding the regulatory domain of SnRK1A between the Ubi promoter and a Nos terminator (Lu et al., 2007). Plasmid p5xUAS-35SminiP-Luc-Nos contains 5 tandem repeats of UAS fused to the upstream of CaMV35S minimal promoter-Adh1 intron-Luc fusion gene (Lu et al., 1998). pAHC contains the Luc cDNA between the Ubi promoter and the Nos terminator (Bruce et al., 1989).

### Yeast Two-Hybrid Assay

For cloning of SnRK1A-interacting proteins, a yeast (Saccharomyces cerevisiae) two-hybrid cDNA library was constructed by fusion of cDNAs, which were derived from poly(A) mRNAs isolated from rice suspension cells starved of sucrose for 8 hours, with the GAL4 activation domain (GAD) DNA in the phagemid vector pAD-GAL4-2.1. Approximately 1×106 transformants were subjected to the two-hybrid selection on a synthetic complete (SC) medium lacking leucine, tryptophan, and histidine but containing 15 mM 3-amino-1,2,4-triazole (3-AT). The expression of the HIS3 reporter gene allowed colonies to grow on the selective medium, and putative positive transformants were tested for the induction of other reporter genes, such as lacZ. Positive colonies were assessed by re-transformation into yeast, and cDNA inserts were identified by DNA sequencing analysis.

For studying the interaction between SnRK1A and SKIN, a Yeastmarker™ Transformation System 2 was used as described by the manufacturer (Clontech, USA). The two-hybrid assay was carried out in yeast (S. cerevisiae) strains AH109 and Y187 (Clontech) that contain reporter genes HIS3 and lacZ under the control of a GAL4-responsive element (Chien et al., 1991). Colonies were grown on selective medium and tested for β-galactosidase activity by a colony-lift filter assay method (Breeden and Nasmyth, 1985).

### Plasmid Construction

The GATEWAY gene cloning system (Invitrogen, USA) was used to generate all constructions. First, destination vectors that could be used in all of experiment were generated. For constructs used in the rice embryo transient expression assay, plasmid pAHC18 was digested with BamHI to remove the luciferase cDNA insert followed by the addition of a double-HA tag, generating pAHC18-2HA. pAHC18-2HA was linearized with EcoRV and inserted with ccdB DNA fragment flanked by attR1 and attR2 between the Ubi promoter and Nos terminator, generating the destination vector pUbi-2HA-ccdB-Nos. For constructs used in the rice stable transformation, pUbi-2HA-ccdB-Nos was linearized with HindIII and inserted into the binary vector pSMY1H (Ho et al., 2000) which has been linearized with the same restriction enzyme, generating the destination vector pSMY1H-pUbi-2HA-DEST-Nos.

For constructs used in the yeast two-hybrid assay, pAS2-1 containing the ADH1 promoter fused to the Gal4 binding domain DNA (ADH1-GAD) and pGAD424 containing the ADH1 promoter fused to Ga14 activation domain DNA (ADH1-GBD) were linearized with SmaI, and the ccdB DNA fragment flanked by attR1 and attR2 sties was inserted downstream of ADH1-GAD or ADH1-GBD, generating destination vectors GAD-ccdB and GBD-ccdB. The coding sequence of SKIN1, SKIN2 and SnRK1A (wild type or truncated) were synthesized by PCR and inserted between the attL1 and attL2 sites in pENTR™/Directional TOPO Cloning Kits (Invitrogen, USA), generating pENTR-SKIN and pENTR-SnRK1A. Various genes fused at C-termini of GAD and GBD were driven by the ADH1 promoter through the GATEWAY lambda recombination system (LR Clonase II enzyme mix kit, Invitrogen).

For the SKIN RNA interference (RNAi) construct, two 307- and 245-bp fragments respectively derived from the 3′UTR of SKIN1 and SKIN2 cDNA were synthesized by PCR. Either of them is fused in antisense and sense orientations flanking the 750-bp GFP cDNA. The SKIN RNAi fragments were inserted between the attL1 and attL2 sites in pENTR/D-TOPO, generating pENTR-SKIN-Ri. Through the GATEWAY lambda recombination system (LR Clonase II enzyme mix kit, Invitrogen), generating the entry vector pENTR-SKIN(Ri), and through the GATEWAY lambda recombination system to generate pSMY1H-SKIN-Ri, including pSMY1H-SKIN1-Ri and pSMY1H-SKIN2-Ri.

The 307-bp fragment derived from the 3′UTR of SKIN/(SEQ ID No: 58):

GCTATTAGTACAAAAAAAATAATAATTTTTACAGTTAGAGCAAAAAGCCA
TTGATCTCCTTTTGGCTGGTAGAGTTGTTACTGCTACAACTGCTTACTAT
TAGTAACTATATAATTATAATTATAATTGCAATGCATAAGGTCCAAGTTT
GTTGTGATCTACTATGATTCTAGTAACTCTCTGGTTTTTCTGAGTCCTGA
CCTGATTAAGAAGACATGTATCAACTATGTATATCTATGAACTGACCTAA
CTTGAGGCTATCATTAACTAATGATGGTTTATGATTAGTCAATTGCTTTG
CTTTTGA

The 245-bp fragment derived from the 3′UTR of SKIN2 (SEQ ID No: 59):

CTCAAGAAAAAAAAATCTAGGTTTCTGCTTCTTCTCTTGTCTGAAAATTT
TAGGGGTGTGAGAGAAATCATCAGTGTTGTTGTTACTGCTGCTGCTGCTG
CTATATGATCAAGATATATATAACAAAAAAAAAGAACTCCATTTGTTTGT
GTGCTTGTCTCTGGATGAACTCTGATCTTGATGATGATGATGAATCTTGT
CTGTCTGGCATGAGGTCAACAACTCAACATTGCTATGAACAAAAA

The 750-bp fragment derived from the cDNA of GFP (SEQ ID No: 60):

atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccttcaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacgggagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactcacggca
tggacgagctgtacaagtctagataggagatccgtcgacctgcagatcgt

For protein cellular localization, the full-length SKIN cDNA was inserted between the attL1 and attL2 sites in pENTR/D-TOPO, generating the entry vector pENTR-SKIN. SKIN in pENTR-SKIN was then inserted downstream of pUbi-2HA in pSMY1H-pUbi-2HA-DEST-Nos through the GATEWAY lambda recombination system, generating pSMY1H-Ubi-2HA-SKIN-Nos.

For construction of SKIN without NLS (SKINΔNLS), SKIN cDNA lacking DNA encoding the NLS (KRRR) was inserted between the attL1 and attL2 sites in pENTR/D-TOPO, generating the entry vector pENTR-SKINΔNLS. SKINΔNLS in pENTR-SKINΔNLS was then inserted downstream of pUbi-2HA in pUbi-2HA-DEST-Nos through the GATEWAY lambda recombination system, generating pUbi-2HA-SKINΔNLS-Nos, and also inserted downstream of pUbi-GFP in pUbi-GFP-DEST-Nos, generating pUbi-GFP-SKINΔNLS-Nos.

### Rice Transformation

Plasmids for overexpressing SKIN1 and SKIN2 (i.e. pSMY1H-pUbi-2HA-SKIN, to including pSMY1H-Ubi-2HA-SKIN1-Nos and pSMY1H-Ubi-2HA-SKIN2-Nos) and plasmids for silencing SKIN1 and SKIN2 (i.e. pSMY1H-SKIN-Ri, including pSMY1H-SKIN1-Ri and pSMY1H-SKIN2-Ri) were separately introduced into Agrobacterium tumefaciens strain EHA105, and rice transformation was performed as described (Ho et al., 2000). Many transgenic lines were obtained after transformation, in which (SKIN2-Ox)O2, (SKIN1-Ox)O3, (SKIN1-Ri)R3, (SKIN2-Ri)R1 were selected for the following experiments because their overexpression or silencing effect are better. In addition, other SKIN-Ox lines (O6) and SKIN-Ri lines (R2, R5) were also used.

### Rice Embryo and Barley Aleurone Transient Expression Assays

Rice embryos were prepared for particle bombardment as described (Chen et al., 2006). The rice embryos were bombarded with reporter, effectors and internal control at a ratio of 4:2:1 for single effector or 4:2:2:1 for two effectors. The internal control (Ubi::GUS) was used to normalize the reporter enzyme activity because different transformation efficiency might occur in each independent experiment. Bombarded rice embryos were divided into two halves, with half being incubated in MS liquid medium containing 100 mM Glc, and the other half grown in MS liquid containing 100 mM mannitol, for 24 h. Total proteins were extracted for embryos with a cell lysis buffer [0.1 M K-phosphate, pH 7.8, 1 mM EDTA, 10% glycerol, 1% triton X-100, and 7 mM β-mercaptoethanol]. GUS assay buffer [0.1 M Na-phosphate, 20 mM EDTA, 0.2% sarcosine, 0.2% Triton X-100, and 20 mM β-mercaptoethanol] was used for GUS activity assay. The activity assay of GUS and luciferase were described elsewhere (Lu et al., 1998). All bombardments were repeated at least three times.

The barley aleurone/endosperm transient expression assays were performed as described (Hong et al., 2012). Each independent experiment consisted of three replicates, with six endosperms for each treatment, and was repeated three times with similar results. Luciferase and GUS activity assays were performed as described (Hong et al., 2012). Error bars indicate the SE for three replicate experiments.

### Real-Time Quantitative RT-PCR Analysis

Total RNA was extracted from leaves of rice seedlings with the Trizol reagent (Invitrogen) and treated with RNase-free DNase I (Promega, Madison, Wis.). Five to ten μg of RNA was used for cDNA preparation using reverse transcriptase (Applied Biosystems, Foster City, Calif.), and cDNA was then diluted to 10 ng/μl for storage. Five μl of cDNA was mixed with primers and the 2× Power SYBR Green PCR Master Mix reagent (Roche), and applied to an ABI 7500 Real-Time PCR System (Applied Biosystems). The quantitative variation between different samples was evaluated by the delta-delta CT method, and the amplification of 18S ribosomal RNA was used as an internal control to normalize all data.

### Antibodies and Western Blot Analysis

The anti-SnRK1 polyclonal antibodies were produced against synthetic peptides (5′-RKWALGLQSRAHPRE-3′, amino acid residues 385 to 399, SEQ ID No: 70) derived from SnRK1A. Mouse monoclonal antibody against HA tag (Sigma) were purchased. The Western blot analysis was performed as describes (Lu et al., 2007). Horseradish peroxidase-conjugated antibody against rabbit immunoglobulin G (Amersham Biosciences) was used as a secondary antibody. Protein signals were detected by chemiluminescence with ECL (Amersham Bioscience). Ponceau S staining of proteins was used for a loading control.

### Seed Germination in Air or Under Water

The experiment was performed as described (Lee et al., 2009). For germination in air, seeds were placed on 3M filter papers wetted with water in a 50-ml centrifuge tube which contains half-strength MS agar medium. For germination under water, seeds were placed in a 50-ml centrifuge tube, autoclaved water was carefully poured into the tube to avoid any air bubbles, and tubes were sealed with lids.

### Confocal Microscopy for Detection of GFP

Detection of cellular localization of SKIN-GFP, SnRK1A-GFP and MYBS1-GFP fusion proteins were performed as described (Hong et al., 2012). Embryoless barley and rice seeds were sterilized with 1% NaOCl for 30 mins, and incubated in a buffer containing 20 mM CaCl₂ and 20 mM sodium succinate, pH 5.0, for 4 days. Aleurone layers were isolated by scratching away starch in the endosperm with a razor blade. Four aleurone layers were arranged in a 10-cm dish for bombardment. Aleurone layers expressing GFP were examined with a Ziess confocal microscope (LSM510META) using a 488-nm laser line for excitation and a 515- to 560-nm long pass filter for emission.

### Primers

All primer used for the cloning of plasmid constructions are listed in Tables 1 and 2. Primers used for quantitative RT-PCR are listed in Table 3.

TABLE 1
Primer list.

| | | |
|---|---|---|
| Primer | Sequence (5'→3') | Sequence No. |

Plasmid construction

SKIN1

| | | |
|---|---|---|
| SKIN1(F) | CACCATGTCGACGGCGGTGGCGGA | SEQ ID No: 5 |
| SKIN1(R) | ACATGAACCGCCACTGT | SEQ ID No: 6 |
| SKIN13(F) | GCTATTAGTACAAAAAAAAT | SEQ ID No: 7 |
| SKIN13(R) | CACCTCAAAAGCAAAGCAATTGAC | SEQ ID No: 8 |
| SKIN184(F) | CACCGTGGAGAGCAAGCTCAAGGC | SEQ ID No: 9 |
| SKIN183(R) | CTCCTCCTCGTCGTCCCCTC | SEQ ID No: 10 |
| SKIN1159(R) | CGACCAGGTGGCGAGGATGC | SEQ ID No: 11 |
| SKIN1160(F) | CACCCGGCGAGCCTCCTGCAGCTC | SEQ ID No: 12 |
| SKIN1 muGKSKSF(F) | TGCTGCTGCGGCGTAGAAGTTGGAGAGCCCCC | SEQ ID No: 13 |
| SKIN1 muGKSKSF(R) | GCAGCAGCAACCAGCCTCGCCGAGGCGACGGC | SEQ ID No: 14 |
| SKIN1ΔGKSKSF(F) | GGCGTAGAAGTTGGAGAGCCCCCTC | SEQ ID No: 15 |
| SKIN1ΔGKSKSF(R) | ACCAGCCTCGCCGAGGCGACGGCGT | SEQ ID No: 16 |
| SKIN1 muNLS(F) | GGCCGCGTTGAAGGGGTTCTCCG | SEQ ID No: 17 |
| SKIN1 muNLS(R) | GCCGCCATCCTCGCCACCTGGTCGC | SEQ ID No: 18 |

SKIN2

| | | |
|---|---|---|
| SKIN2(F) | CACCATGTCCACGGCGGTGGCGCG | SEQ ID No: 19 |
| SKIN2(R) | GTTATAGGAGCCTGCATTTT | SEQ ID No: 20 |
| SKIN23(F) | AAAATCTAGGTTTCTGCTTC | SEQ ID No: 21 |
| SKIN23(R) | CACCGATTCATCATCATCATCAAG | SEQ ID No: 22 |
| SKIN285(R) | CTCCACCTCCTCCCCCTCCTCCTCC | SEQ ID No: 23 |
| SKIN286(F) | CACCAGCAAGGCGAAGGAGG | SEQ ID No: 24 |
| SKIN2 muNLS(F) | GCCGCCCGTCCTCGCCGCGTGGTCGCGGCG | SEQ ID No: 25 |
| SKIN2 muNL S(R) | CGCCGCGTTGAACGGGTTCTCCGGCTTGG | SEQ ID No: 26 |

SnRK1A

| | | |
|---|---|---|
| SnRK1A(F) | CACCATGGAGGGAGCTGGCAGAGAT | SEQ ID No: 27 |
| SnRK1A(R) | AAGGACTCTCAGCTGAGT | SEQ ID No: 28 |
| SnRK1A 331(R) | GCGCAGCCTATTGTCCAATA | SEQ ID No: 29 |
| SnRK1A 279(R) | AGGAGGTGGCACAGCTAAATAACGCG | SEQ ID No: 30 |
| SnRK1A 280(F) | CACCTGACACTGCACAACAGGTTAAAAAGC | SEQ ID No: 31 |
| GAD(F) | CACCATGGATAAAGCGGAATTAATTCCCGA | SEQ ID No: 32 |
| GBD(F) | CACCATGAAGCTACTGTCTTCTATCGAACA | SEQ ID No: 33 |

TABLE 2
Primer pairs used for plasmid construction.

| | | |
|---|---|---|
| Forward primer | Reverse primer | Product (Plasmid) |

SKIN1

| | | |
|---|---|---|
| SKIN1(F) | SKIN1(R) | SKIN1 1-259 (pUbi-SKIN1-Nos, pAdh1-GBD-SKIN1, pUbi-SKIN1-GFP-Nos) |
| SKIN1(F) | SKIN183(R) | SKIN1 1-83 (pUbi-SKIN1 1-83-Nos, pAdh1-GBD-SKIN1 1-83) |
| SKIN1(F) | SKIN1159(R) | SKIN1 1-159 (pUbi-SKIN1 1-159-Nos, pAdh1-GBD-SKIN1 1-159) |
| SKIN184(F) | SKIN1(R) | SKIN1 84-259 (pUbi-SKIN1 84-259-Nos, pAdh1-GBD-SKIN1 84-259) |
| SKIN184(F) | SKIN1159(R) | SKIN1 84-159 (pUbi-SKIN1 84-159-Nos, pAdh1-GBD-SKIN1 84-159) |
| SKIN1160(F) | SKIN1(R) | SKIN1 160-259 (pUbi-SKIN1 160-259-Nos, pAdh1-GBD-SKIN1 160-259) |
| SKIN13(F) | SKIN13(R) | SKIN1 3′UTR (pUbi-SKIN1 3′UTR-GFP-SKIN1 3′UTR-Nos) |
| SKIN1 | SKIN1 | SKIN1 GKSKSF119-124AAAAAA (pUbi-SKIN1GKSKSF119-124AAAAAA-Nos, |
| muGKSKSF(F) | muGKSKSF(R) | using pUbi-SKIN1-Nos as template) |
| SKIN1 ΔGKSKSF(F) | SKIN1 ΔGKSKSF(R) | SKIN1 ΔGKSKSF (pUbi-SKIN1 ΔGKSKSF-Nos, using pUbi-SKIN1-Nos as template) |
| SKIN1 muNLS(F) | SKIN1 muNLS(R) | SKIN1 muNLS (pUbi-SKIN1 muNLS-GFP-Nos, using pUbi-SKIN1-GFP-Nos as template) |
| GBD(F) | SKIN1(R) | GBD-SKIN1 (pUbi-GBD-SKIN1-Nos, using pAdh1-GBD-SKIN1 as template) |
| GBD(F) | SKIN183(R) | GBD-SKIN1 1-83 (pUbi-GBD-SKIN1 1-83-Nos, using pAdh1-GBD-SKIN1 as template) |
| GBD(F) | SKIN1(R) | GBD-SKIN1 84-259 (pUbi-GBD-SKIN1 84-259-Nos, using pAdh1-GBD-SKIN1 84-259 as |
| | | template) |

SKIN2

| | | |
|---|---|---|
| SKIN2(F) | SKIN2(R) | SKIN2 1-261 (pUbi-SKIN2-Nos, pAdh1-GBD-SKIN2) |
| SKIN2(F) | SKIN285(R) | SKIN2 1-85 (pUbi-SKIN2 1-85-Nos, pAdh1-GBD-SKIN2 1-85) |
| SKIN286(F) | SKIN2(R) | SKIN2 86-261 (pUbi-SKIN2 86-261-Nos, pAdh1-GBD-SKIN2 86-261) |
| SKIN23(F) | SKIN23(R) | SKIN2 3′UTR (pUbi-SKIN2 3′UTR-GFP-SKIN2 3′UTR-Nos) |
| GBD(F) | SKIN2(R) | GBD-SKIN2 (pUbi-GBD-SKIN2-Nos, using pAdh1-GBD-SKIN2 as template) |
| GBD(F) | SKIN285(R) | GBD-SKIN2 1-85 (pUbi-GBD-SKIN2 1-85-Nos, using pAdh1-GBD-SKIN2 as template) |
| GBD(F) | SKIN2(R) | GBD-SKIN2 86-261 (pUbi-GBD-SKIN2 86-261-Nos, using pAdh1-GBD-SKIN2 86-261 as |
| | | template) |
| SKIN2 muNLS(F) | SKIN2 muNLS(R) | SKIN2 muNLS (pUbi-SKIN2 muNLS-GFP-Nos, using pUbi-SKIN2-GFP-Nos as template) |

SnRK1A

| | | |
|---|---|---|
| SnRK1A(F) | SnRK1A(R) | SnRK1A 1-505 (pAdh1-GAD-SnRK1A, p35S-SnRK1A-RFP) |
| SnRK1A(F) | SnRK1A 279(R) | SnRK1A 1-279 (pAdh1-GAD-SnRK1A 1-279) |
| SnRK1A(F) | SnRK1A 331(R) | SnRK1A 1-331 (pAdh1-GAD-SnRK1A 1-331) |
| SnRK1A 280(F) | SnRK1A(R) | SnRK1A 280-505 (pAdh1-GAD-SnRK1A 280-505) |
| GAD(F) | SnRK1A(R) | GAD-SnRK1A (pUbi-GAD-SnRK1A, using pAdh1-GAD-SnRK1A as template) |
| GAD(F) | SnRK1A 279(R) | GAD-SnRK1A KD(1-279) (pUbi-GAD-SnRK1A KD(1-279), using pAdh1-GAD-SnRK1A |
| | | as template) |
| GAD(F) | SnRK1A(R) | GAD-SnRK1A RD(280-505) (pUbi-GAD-SnRK1A RD(280-505), using pAdh1-GAD- |
| | | SnRK1A 280-505 as template) |

TABLE 3
Primer list of quantitative RT-PCR analysis.

| Primer | Sequence (5'→3') | Sequence No. |
|---|---|---|
| 18S(F) | CCTATCAACTTTCGATGGTAGG | SEQ ID No: 34 |
| | ATA | |
| 18S(R) | CGTTAAGGGATTTAGATTGTAC | SEQ ID No: 35 |
| | TCATT | |
| 3RT25A(F) | GTAGGCAGGCTCTCTAGCCTCT | SEQ ID No: 36 |
| | AGG | |
| 3RT(R) | AACCTGACATTATATATTGCAC | SEQ ID No: 37 |
| | C | |
| 8RT1(F) | CTCAGGGTTCCTGCCGGTAGAA | SEQ ID No: 38 |
| | AGCA | |
| 8RTB(R) | CGAAACGAACAGTAGCTAG | SEQ ID No: 39 |
| SKIN1Q(F) | AGAGAGGGAAGCCTGAGGAG | SEQ ID No: 40 |
| SKIN1Q(R) | CTTGAGCTTGCTCTCCACCT | SEQ ID No: 41 |
| SKIN2Q(F) | CTTGACGCCGAGGAGCTCGAAT | SEQ ID No: 42 |
| SKIN2(R) | GCCTGCATTTTGGAGATCGG | SEQ ID No: 43 |
| SnRK1AQ(F) | TTATGCCGTTGTCTGCTTCC | SEQ ID No: 44 |
| SnRK1AQ(R) | CTACTGGAGGATTATGGTCA | SEQ ID No: 45 |
| MYBS1Q(F) | CCATGGACGGACATGAGCAGCA | SEQ ID No: 46 |
| | TTT | |
| MYBS1Q(R) | AAGATGATCAGGGACGATGA | SEQ ID No: 47 |
| GIF1Q(F) | CATCGCGCAACCCGAACATG | SEQ ID No: 48 |
| GIF1Q(R) | TGTCGATCAGGCTCCTCAGAG | SEQ ID No: 49 |
| STQ(F) | TGAGCCAGCTCTCATCCTGC | SEQ ID No: 50 |
| STQ(R) | GAGCCGATAGAAACTGAGGG | SEQ ID No: 51 |
| Lip1Q(F) | TGCAGATTACGCTAATTCAT | SEQ ID No: 52 |
| Lip1Q(R) | CCTCTTATAGCTAACTTTAGC | SEQ ID No: 53 |
| EP3AQ(F) | CGCCTACGAGCCTGGATCAA | SEQ ID No: 54 |
| EP3AQ(R) | TAAACACAAGGCAATTAACA | SEQ ID No: 55 |
| Phospho1Q | AAACGGCTAGCTCGAACAAT | SEQ ID No: 56 |
| (F) | | |
| Phospho1Q | CTAATCGCAGGCTCAATCAC | SEQ ID No: 57 |
| (R) | | |

### Accession Numbers

SKIN1 (AK060116); SKIN2 (AK072516); SnRK1A (AB101655.1); MYBS1 (AY151042.1); αAmy3/RAmy3D (M59351.1); αAmy8/RAmy3E (M59352.1), EP3A encoding Cys protease (AF099203); Lip1 encoding GDSL-motif lipase (AK070261); Phospho1 encoding phosphatase-like (AK061237); ST encoding sugar transporter family protein (AK069132); ZmMTD1 (ACG28615.1); ZmKCP (ZAA48125.1); Sorghum02g028960 (XP_{—}002462609.1); AtKCP (NC_{—}003076.8); AtKCL1 (NC_{—}003075); AtKCL2 (NC_{—}003071); BnKCP1 (AY211985); ZmMTD186T7R4 (EU961029)

### Results

A Novel SKIN Family Interacts with SnRK1A

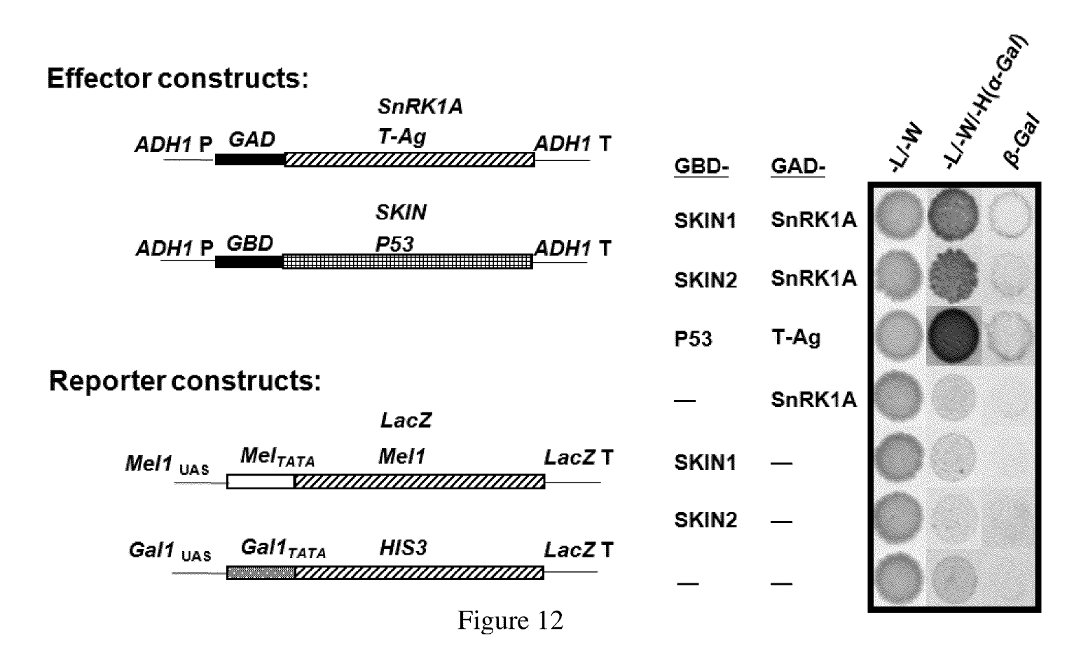

To identify components that interact with SnRK1A, we performed a yeast two-hybrid screen. The full-length cDNA of SnRK1A was fused to the Ga14 activation domain DNA (GAD-SnRK1A) and used as bait for screening a rice cDNA library derived from sucrose-starved rice suspension cells. One gene encoding a novel protein was identified and the protein was designated as the SnRK1A-interacting negative regulator 1 (SKIN1). Bioinformatics analysis of the rice genome also identified a SKIN1 homolog that was designated as SKIN2. The interaction between SKIN fused to the Ga14-binding domain (GBD-SKIN) and GAD-SnRK1A was analyzed using the yeast two-hybrid assay. Both SKIN1 and SKIN2 interacted with SnRK1A in yeast (FIG. 12).

The nucleotide sequence of SKIN1 is shown below:

SEQ ID NO: 1
ATGTCGACGGCGGTGGCGGACGTGCCACCGGCGGCGGCCTACGGGTTCCC
CGGATCGGCCAAGAGAGGGAAGCCTGAGGAGGTGGTGGTGCTGATGGGGA
AGAGGAGGAACGAAGGGTTCTTCATCGAGGAGGAGGAGGAGGAGGAGGAG
GTGCTGACGGAGAGCTCGTCGATCGGCGCGCCGTCGCCGGCGAGCTCGTC
GATCGGGGAGAACTCCGGCGAGGAGGAGGGAGGGGACGACGAGGAGGAGG
TGGAGAGCAAGCTCAAGGCGGAGGATGAGCAGGTCGGCCTCGGCTGCTTG
GACGCCTTGGAGGAATCCTTACCCATCAAGAGGGGGCTCTCCAACTTCTA
CGCCGGCAAGTCCAAGTCGTTCACCAGCCTCGCCGAGGCGACGGCGTCGC
CGGCGGCGGCGGCCAACGAGCTGGCCAAGCCGGAGAACCCCTTCAACAAG
CGCCGCCGCATCCTCGCCACCTGGTCGCGGCGAGCCTCCTGCAGCTCCCT
CGCCACCGCCACCTACCTCCCACCTCTCCTCGCGCCCGACCACGCCGTCG
CCGAGGGCGACGAGGGTGAGGAGGAAGACGACGATTCAGACGACGATGAG
CGGCAGCACCGTGGCAAGAACGGCGGCCGGCGAGAGTCGGCGGCGCCGCC
ATTGCCATTGCCGCCGCCGAGGCTCACCTTGCACACCCAGATGGGAGGAA
TGGTGAGGAGGAATGGAACATTCAGGTCGCCGAGGTCGCTCTCACTGTCT
GATCTTCAGAACAGTGGCGGTTCATGTTAG

The amino acid sequence of SKIN1 is shown below:

SEQ ID NO: 2
Mstavadvppaaaygfpgsakrgkpeevvvlmgkrrnegffieeeeeeee
vltesssigapspasssigensgeeeggddeeevesklkaedeqvglgcl
daleeslpikrglsnfyagksksftslaeataspaaaanelakpenpfnk
rrrilatwsrrascsslatatylppllapdhavaegdegeeedddsddde
rqhrgknggrresaapplplppprltlhtqmggmvrrngtfrsprslsls
dlqnsggsc

The nucleotide sequence of SKIN2 is shown below:

SEQ ID NO: 3
ATGTCCACGGCGGTGGCGCGCGGCGGGATGATGCCGGCGGGGCACGGGTT
CGGGAAGGGGAAGGCGGCGGCGGTGGAGGAGGAGGAGGATGAGGTGAACG
GGTTCTTCGTGGAGGAGGAGGAGGAGGAGGAGGAGGAGGAGGAGGCGGCG
GTGTCGGATGCGTCGTCGATCGGGGCGGCGTCGTCGGACAGCTCGTCGAT
CGGGGAGAACTCGTCGTCGGAGAAGGAGGGGGAGGAGGAGGGGGAGGAGG
TGGAGAGCAAGGCGAAGGAGGTGGCGGTGGAGGTGGAGGGAGGGGGGCTC
GGGTTCCATGGATTGGGGACTCTCGAATCCCTGGAGGACGCCCTTCCCAT
CAAGAGGGGACTCTCCAACTTCTACGCCGGCAAGTCCAAGTCGTTCACGA
GCCTGGCCGAGGCGGCGGCGAAGGCGGCGGCGAAGGAGATCGCCAAGCCG
GAGAACCCGTTCAACAAGCGCCGCCGCGTCCTCGCCGCGTGGTCGCGGCG
GCGCGCGTCCTGCAGCTCGCTGGCCACCACCTACCTGCCCCCTCTCCTCG
CCCCCGACCACGCCGTCGTCGAGGAGGAGGACGAGGAGGACGACTCCGAC
GCCGAGCAGTGCAGCGGCAGCGGCGGCGGCAACCGCCGGCGCGAGCCGAC
GTTCCCGCCGCCGAGGCTGAGCCTGCACGCGCAGAAGAGCAGCTTGACGC
CGAGGAGCTCGAATCCGGCGTCGTCGTTTAGATCTCCTAGGTCATTCTCA
CTATCCGATCTCCAAAATGCAGGCTCCTATAACTAG

The amino acid sequence of SKIN2 is shown below:

SEQ ID NO: 4
Mstavarggmmpaghgfgkgkaaaveeeedevngffveeeeeeeeeeeaa
vsdassigaassdsssigensssekegeeegeeveskakevavevegggl
gfhglgtlesledalpikrglsnfyagksksftslaeaaakaaakeiakp
enpfnkrrrvlaawsrrrascsslattylppllapdhavveeedeeddsd
aeqcsgsgggnrrreptfppprlslhaqkssltprssnpassfrsprsfs
lsdlqnagsyn

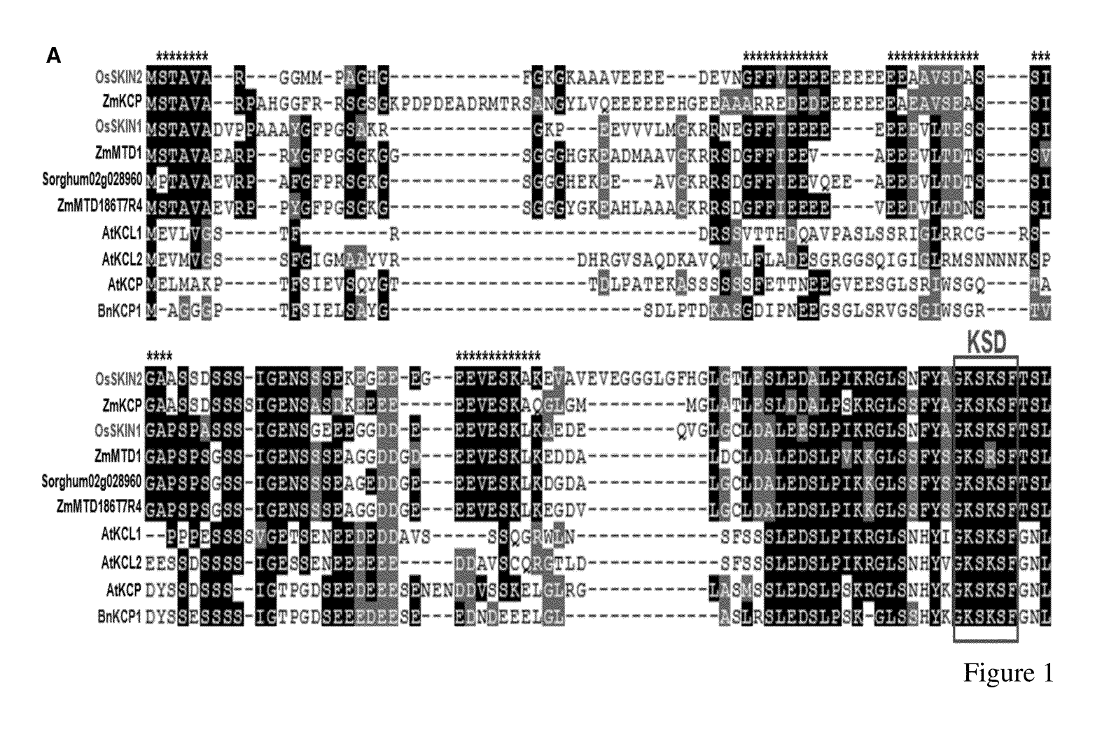

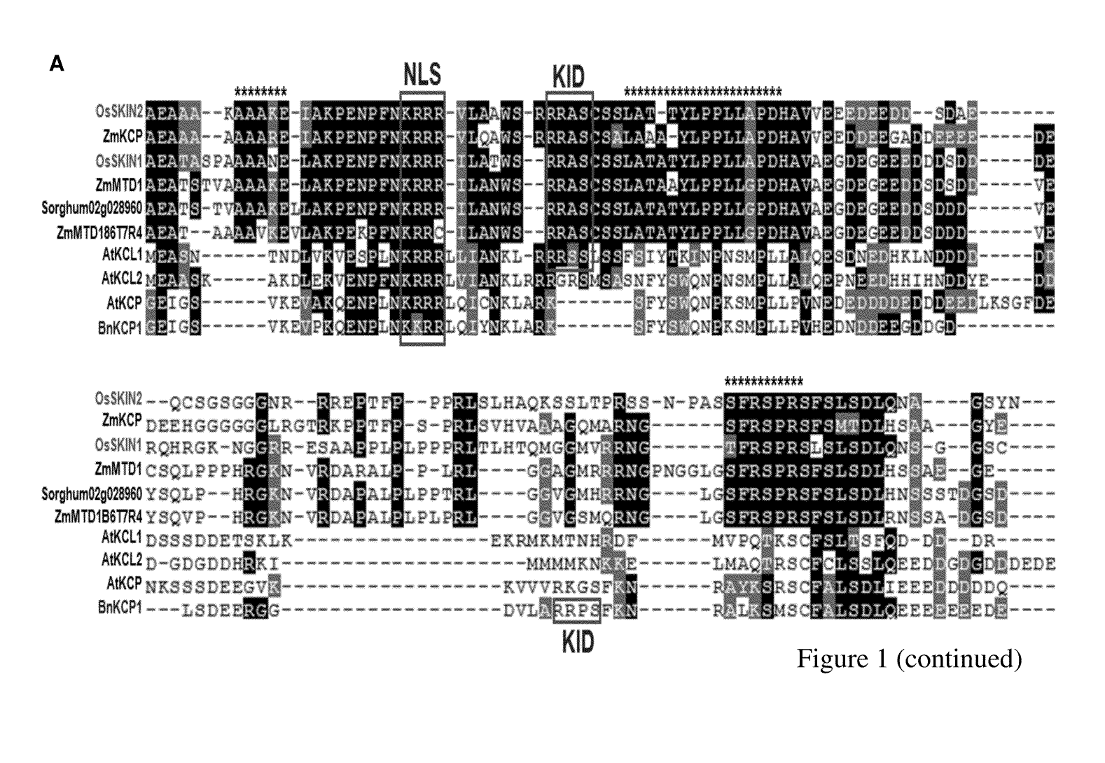

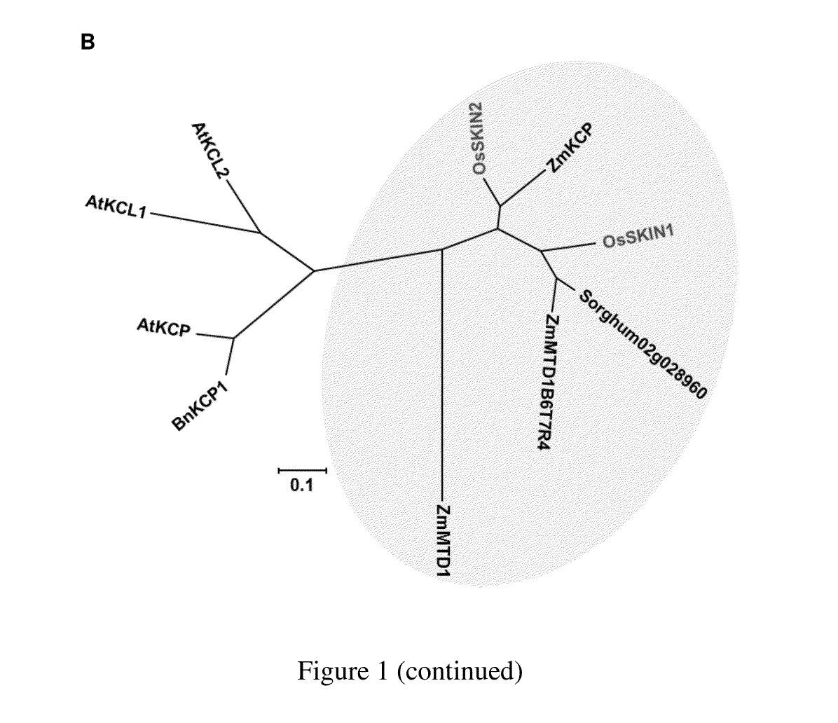

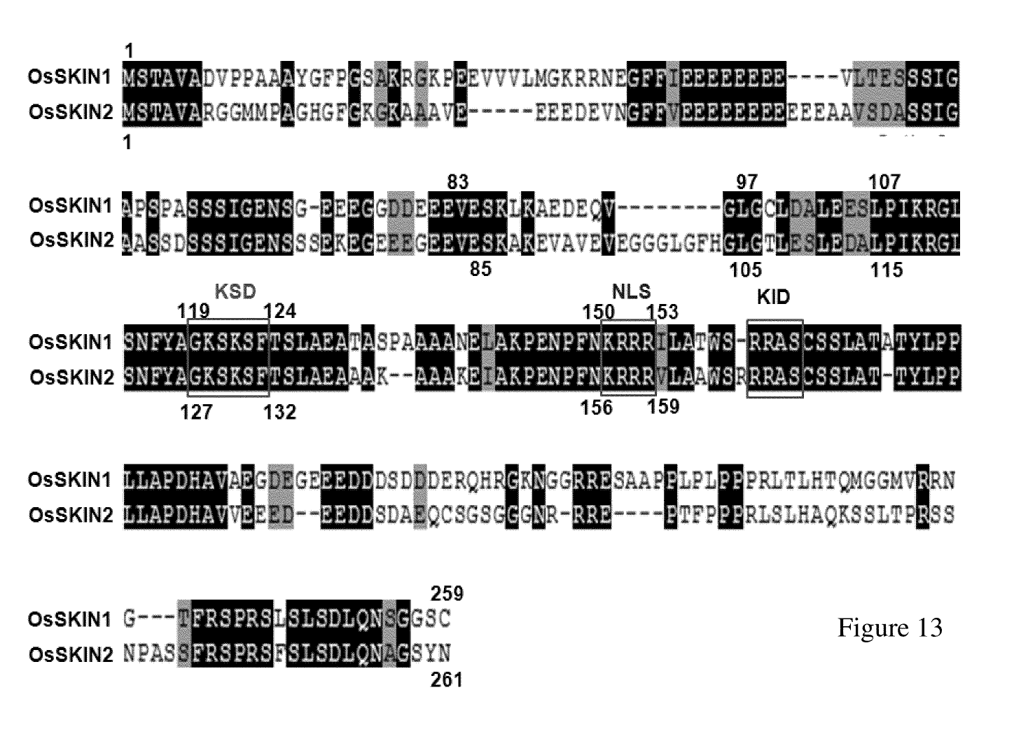

Amino acid sequences of the two SKINs share 59% identity and 69% similarity (FIG. 13). Bioinformatics analysis identified a highly conserved GKSKSF domain (KSD) present in SKIN1 and SKIN2 as well as in several other related proteins from various plant species (FIG. 1A and FIG. 213 Additional conserved domains in these proteins include a putative nuclear localization signal (NLS) and protein kinase A inducible domain (KID)-like sequence (FIG. 1A). Among these genes, only a KID-domain containing protein from Brassica napus (BnKCP1) has been characterized. BnKCP1 is a nucleus-localized protein that interacts with a histone deacetylase in Arabidopsis (HDA19) via its C-terminal phosphorylated KID domain, and Ser¹⁸⁸ within the KID domain is necessary for the interaction with HDA19 and activation of downstream genes in response to cold stress and inomycin treatment (Gao et al., 2003). Amino acid sequences of SKINs share 40% identity and 54% similarity with BnKCP1. The phylogenetic tree analysis of amino acid sequences indicates that all KSD-containing proteins could be classified into monocot and dicot clusters (FIG. 1B).

The N-Terminal Region of SKIN Interacts with the Kinase Domain of SnRK1A

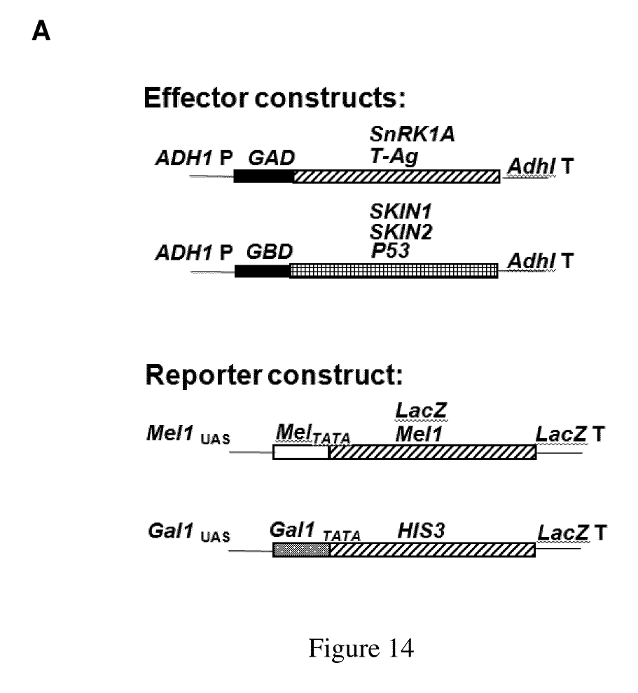

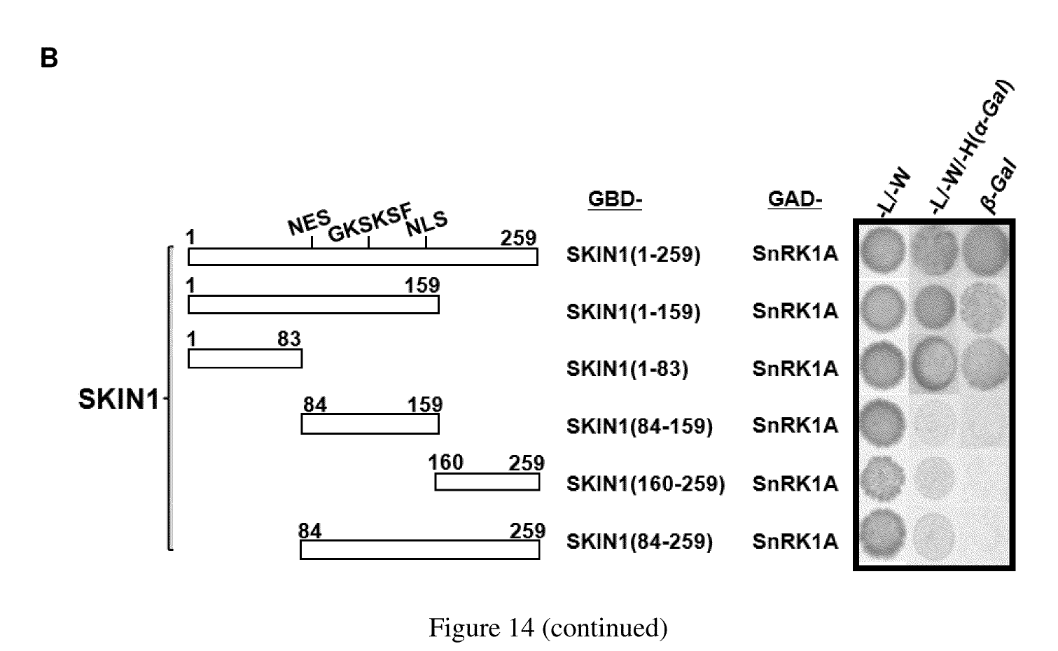

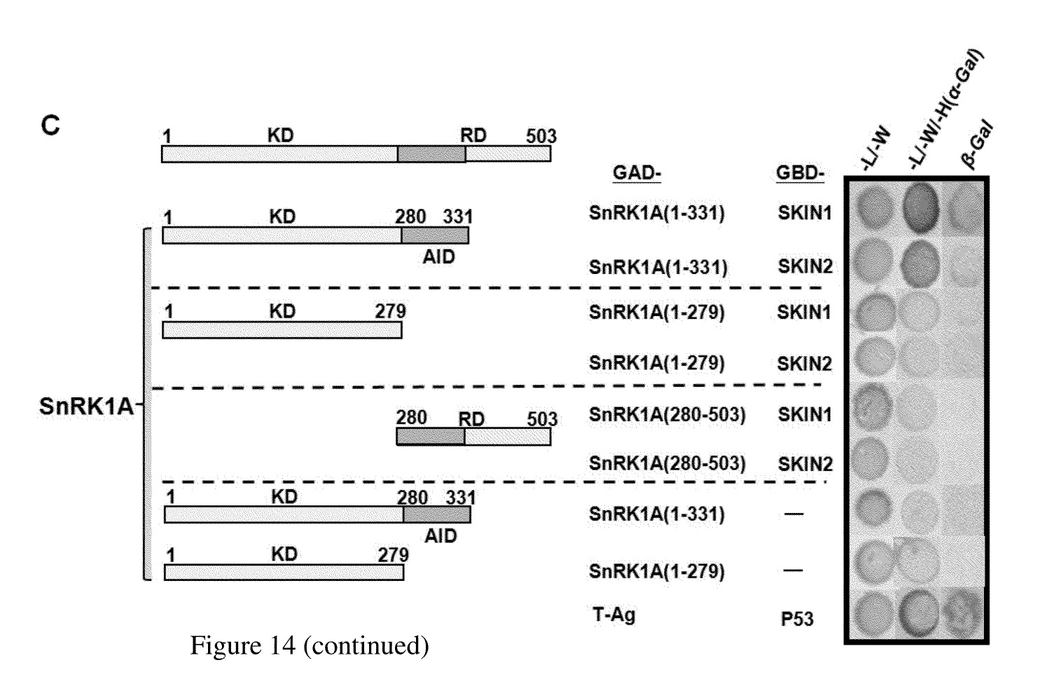

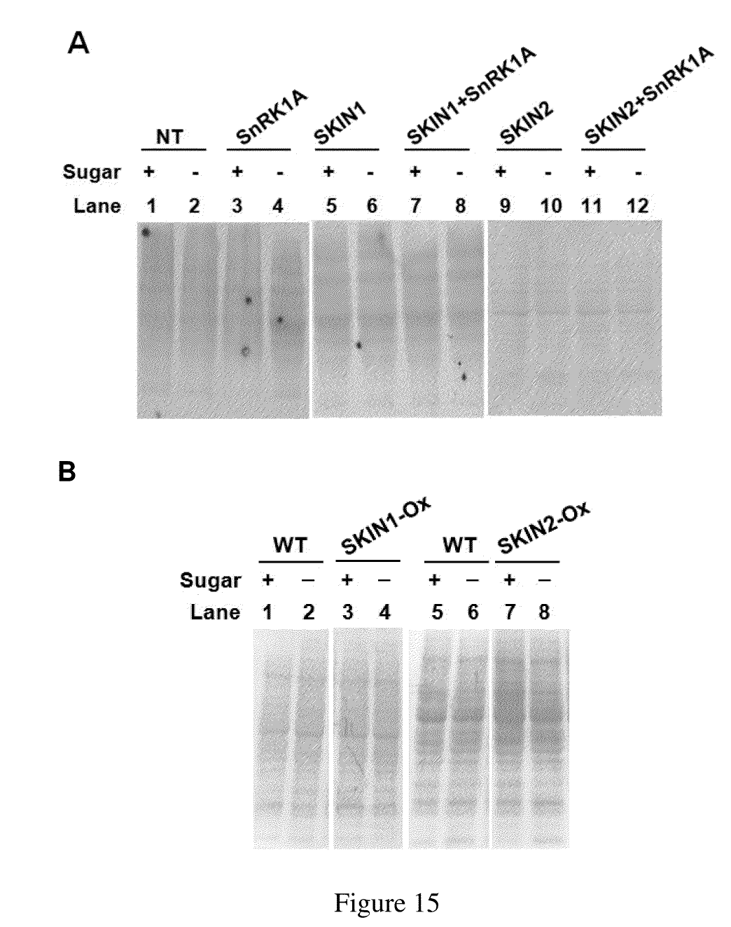

To map the functional domain of SKINs that interact with SnRK1A, five truncated versions of SKIN1 were fused with GBD and analyzed with the yeast two-hybrid assay (FIG. 14A). SKIN1 was truncated to contain amino acids 1-83, which were predicted as a putative coiled-coiled domain by a bioinformatics program, and amino acids 1-159, which ends at the 5′ of the KID domain. All truncated SKIN1 cDNAs lacking amino acids 1-83 did not, whereas amino acids 1-83 by itself could, interact with SnRK1A in yeast (FIG. 14B), indicating SKIN1(1-83) is sufficient and necessary for interaction with SnRK1A in yeast.

To map the domain in SnRK1A that interacts with SKIN in the yeast two-hybrid assay, SnRK1A(1-279) containing the kinase domain (KD), SnRK1A(1-331) containing the KD and the auto-inhibitory domain (AID), and SnRK1A(280-503) containing the regulatory domain (RD) (Lu et al., 2007) were fused with GAD. Only the full-length SnRK1A and SnRK1A(1-331) could interact with SKIN1 and SKIN2 (FIG. 14C), indicating that the KD and AID are sufficient and necessary for interaction with SKINs in yeast.

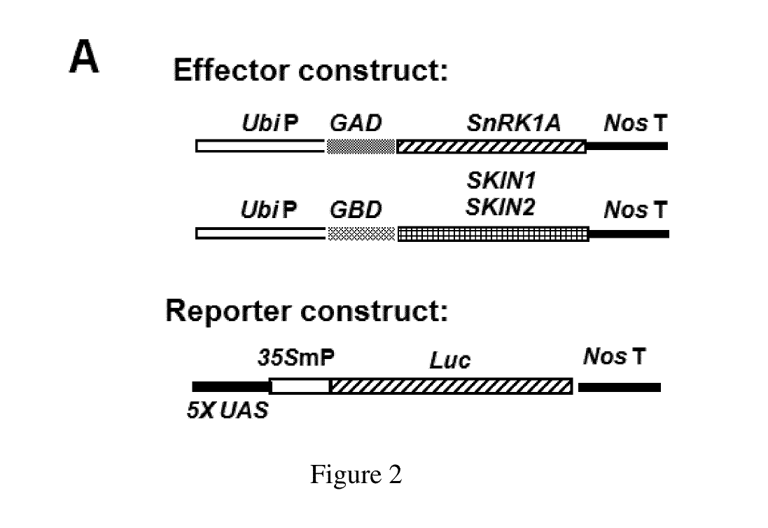

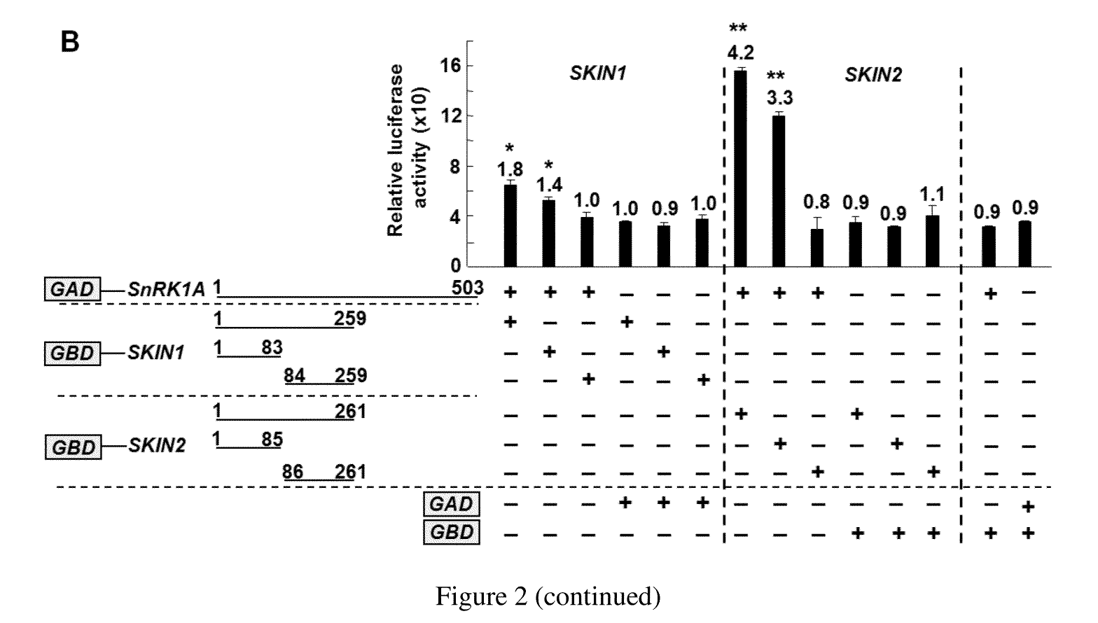

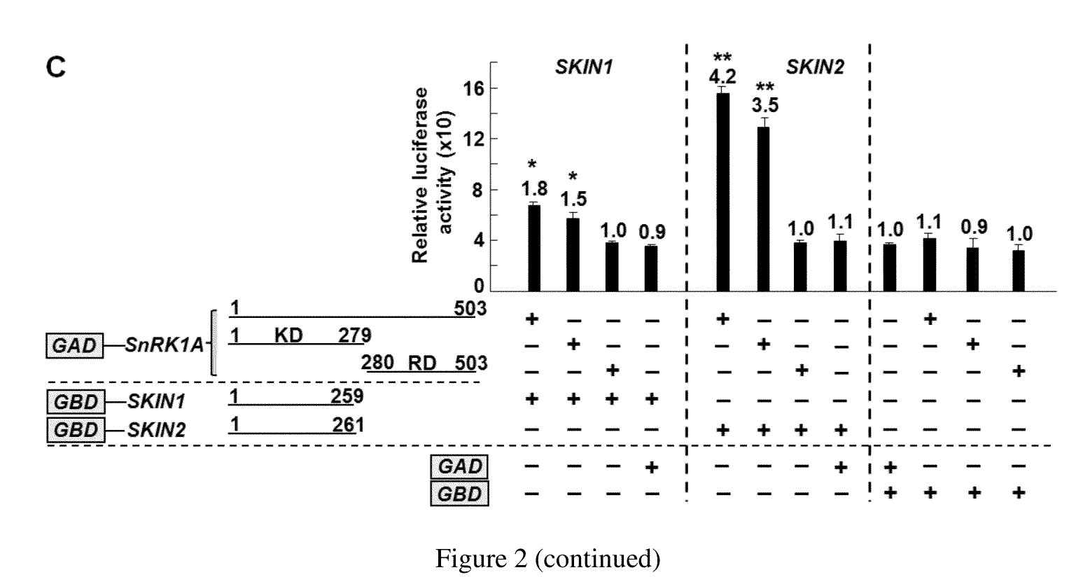

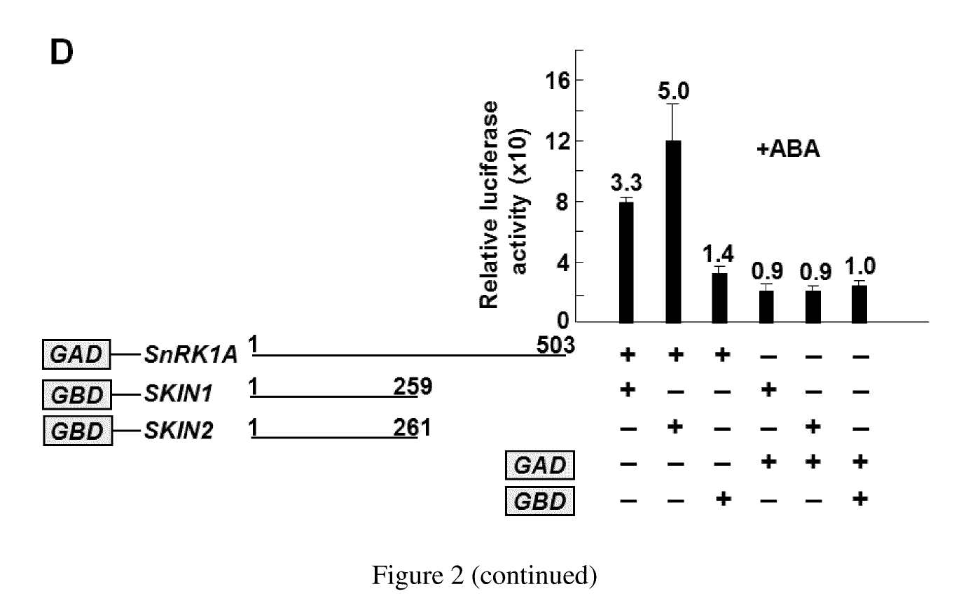

To further demonstrate the physical interaction of SKIN and SnRK1A in planta, a rice embryo two-hybrid assay was employed. Truncated SKIN1 and SKIN2 fused to GBD and expressed under the control of the Ubi promoter served as effectors, and five tandem repeats of the upstream activation sequence (UAS) fused upstream of the CaMV35S minimal promoter—luciferase (Luc) cDNA (5xUAS:Luc) served as a reporter (FIG. 2A). Luciferase activity was enhanced by co-expression of SnRK1A with each of all SKIN1 truncated versions except SKIN1 (84-259) and SKIN2 (86-261) lacking N-terminal regions (FIG. 2B). The functional domain in SnRK1A that interacts with SKIN was also demonstrated in planta. The full-length and KD of SnRK1A interacted with both SKIN1 and SKIN2 (FIG. 2C), which is different from the result of yeast two-hybrid studies in which both KD and AID were required for interaction with SKIN1 and SKIN2 (FIG. 14C). These data confirmed the physical interaction between SKINs and SnRK1A in rice cells, and the N-terminal amino acids 1-83 and 1-85 of SKIN1 and SKIN2, respectively, interact with the KD of SnRK1A.

### The Highly Conserved GKSKSF Domain (KSD) is Necessary for SKINs to Antagonize the Function of SnRK1A

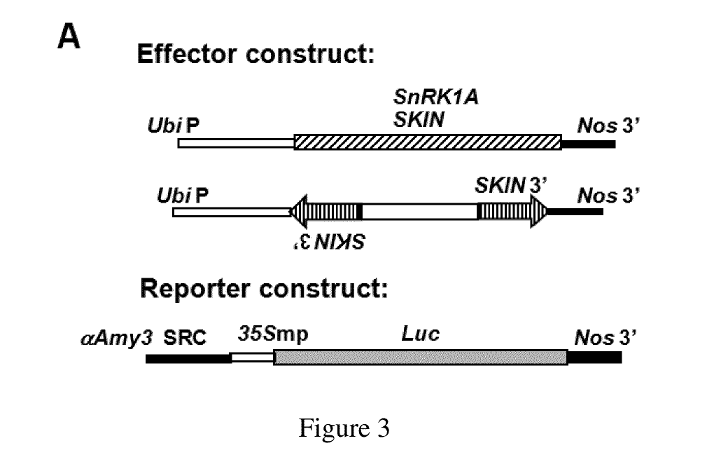

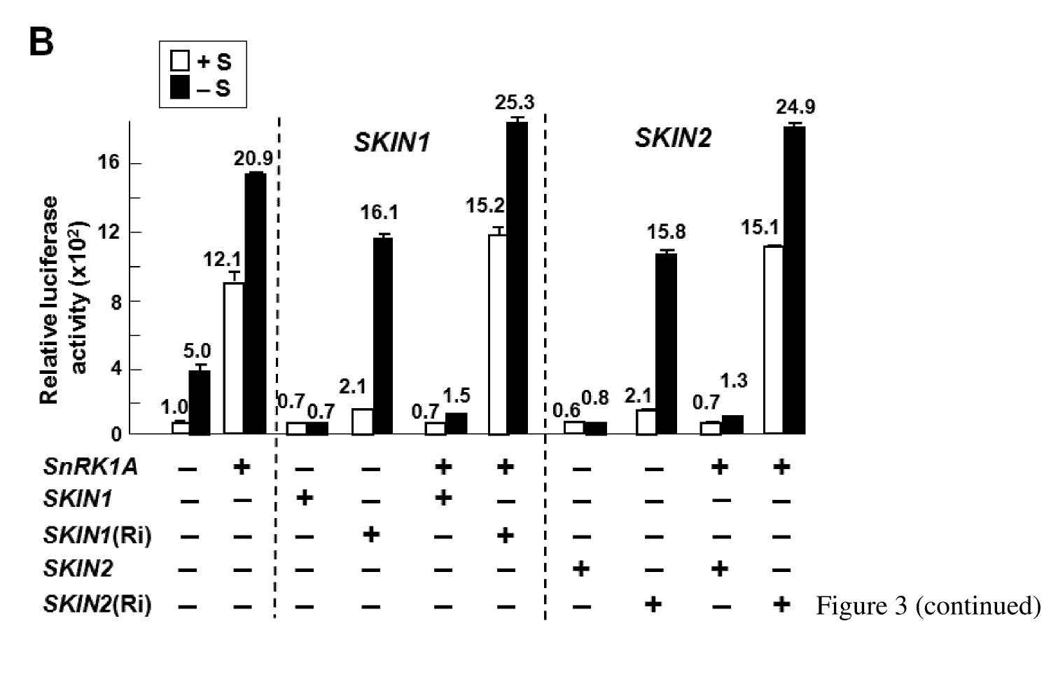

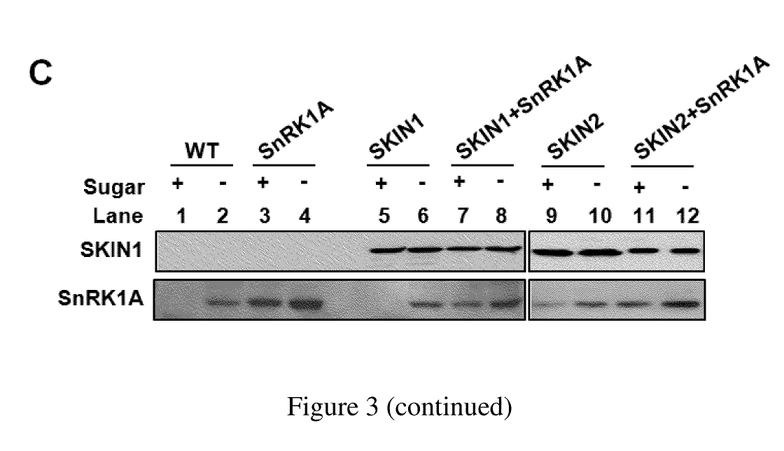

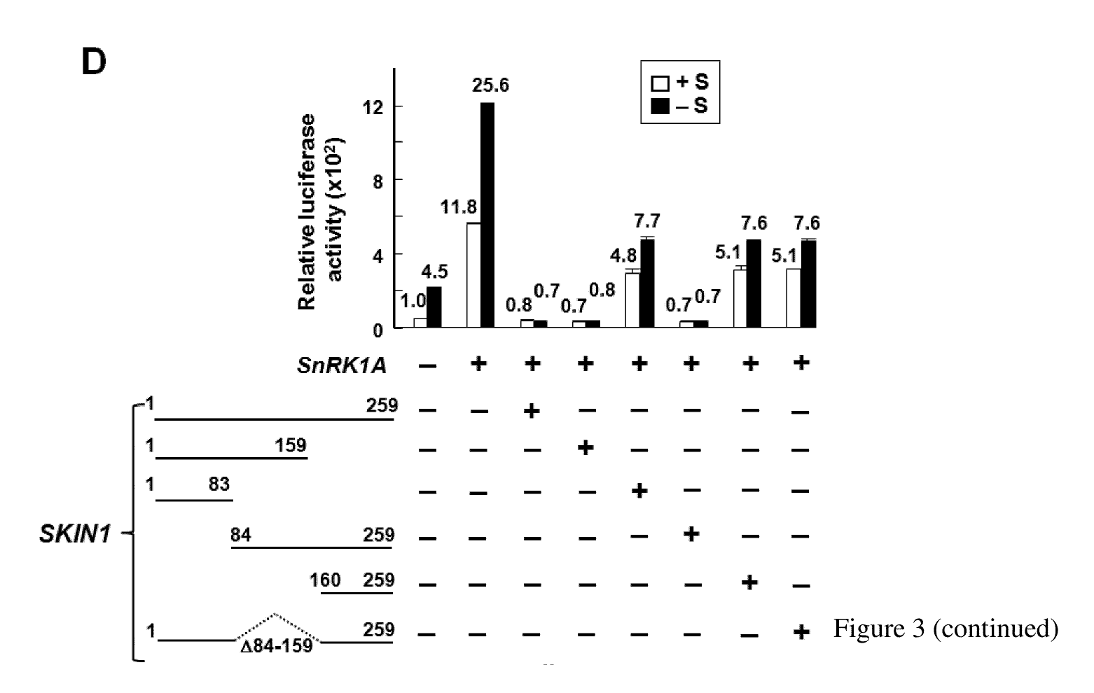

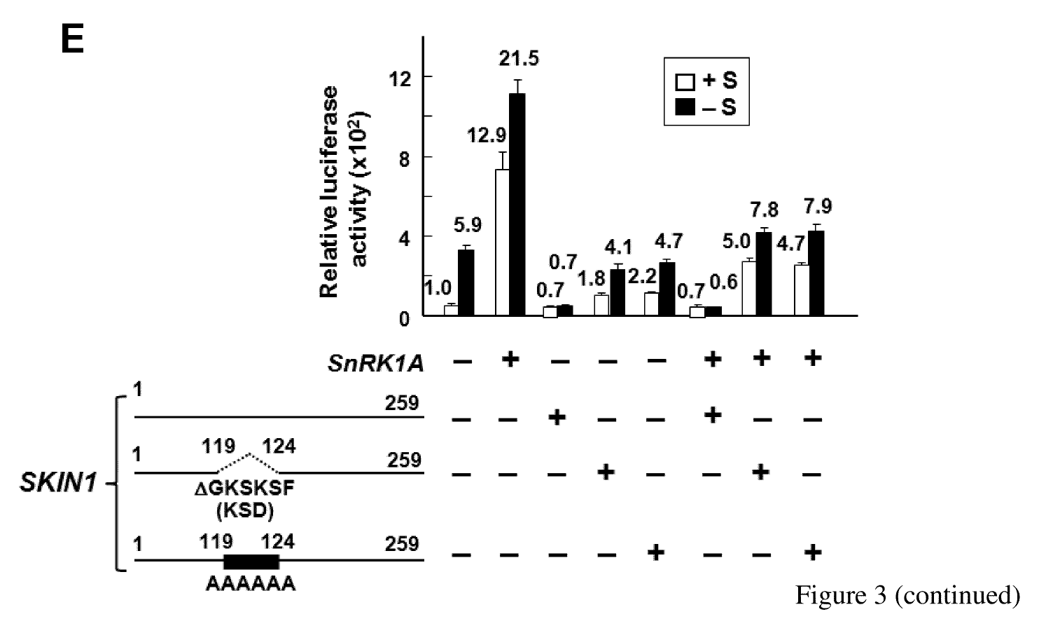

The role of SKIN in the regulation of SnRK1A function was first investigated by gain- and loss-of-function analyses using a rice embryo transient expression assay. SnRK1A and SKIN cDNAs and SKIN RNA interference (Ri) construct expressed under the control of the Ubi promoter served as effectors, and αAmy3 SRC fused to the CaMV35S minimal promoter and Luc cDNA (SRC-35Smp:Luc) as a reporter (FIG. 3A). Overexpression of SnRK1A enhanced, SKINs repressed, while SKIN(Ri) de-repressed the αAmy3 SRC promoter under 100 mM glucose (+S) or or without glucose (−S) conditions for 24 h (FIG. 3B). Co-overexpression of SKIN with SnRK1A repressed the αAmy3 SRC promoter to a level similar to overexpression of SKIN alone, while co-overexpression of SKIN(Ri) with SnRK1A significantly enhanced the αAmy3 SRC promoter under +S and −S conditions (FIG. 3B). These results indicate that SKINs act antagonistically to the SnRK1A-activated αAmy3 expression.

The accumulation of endogenous SnRK1A in non-transfected rice embryos was increased under sugar starvation (FIG. 3C, lanes 1 and 2) as reported previously (Lu et al., 2007). Transient overexpression of SKINs alone or with SnRK1A did not alter the level of SnRK1A accumulation, except the recombinant SnRK1A increased the level of total SnRK1A (FIG. 3C, lanes 5-12), indicating that SKINs antagonize the activity instead of affecting the protein accumulation of SnRK1A.

To further understand the mechanism of SKIN antagonism on SnRK1A function, the functional domain in SKIN that antagonizes SnRK1A activity was investigated. Wild type and truncated versions of SKIN1 expressed under the control of Ubi promoter were used as effectors and SRC-35Smp:Luc as the reporter (FIG. 3A). SKIN1(1-83) and SKIN1(160-259) did not antagonize the function of SnRK1A (FIG. 3D), indicating that the region resides within amino acids 84-159 of SKIN1 might be responsible for antagonism of SnRK1A function. This notion was further confirmed by the loss of inhibitory effect of SKIN1 with international deletion of amino acids 84-159 (FIG. 3D).

Because the highly conserved KSD happens to reside within amino acids 84-159 of SKIN1 (FIG. 13), the KSD was deleted from SKIN1 or replaced with six Ala. Both mutated versions of SKIN1 lost their inhibitory effects on α-Amy3 SRC promoter (FIG. 3E). It is interesting to note that SKINs missing amino acid 84-159 or the KSD domain actually enhanced the function of SnRK1A under both +S and −S conditions (FIGS. 3D and 3E), suggesting that these truncated versions of SKIN might function as dominant negative regulators of the endogenous SKIN. Nevertheless, these studies demonstrated that SKINs are negative regulators of SnRK1A, and the KSD in SKINs is necessary for SKINs to play a role in this repression.

### SKINs Repress the SnRK1A-Dependent Sugar and Nutrient Starvation Signaling Pathway

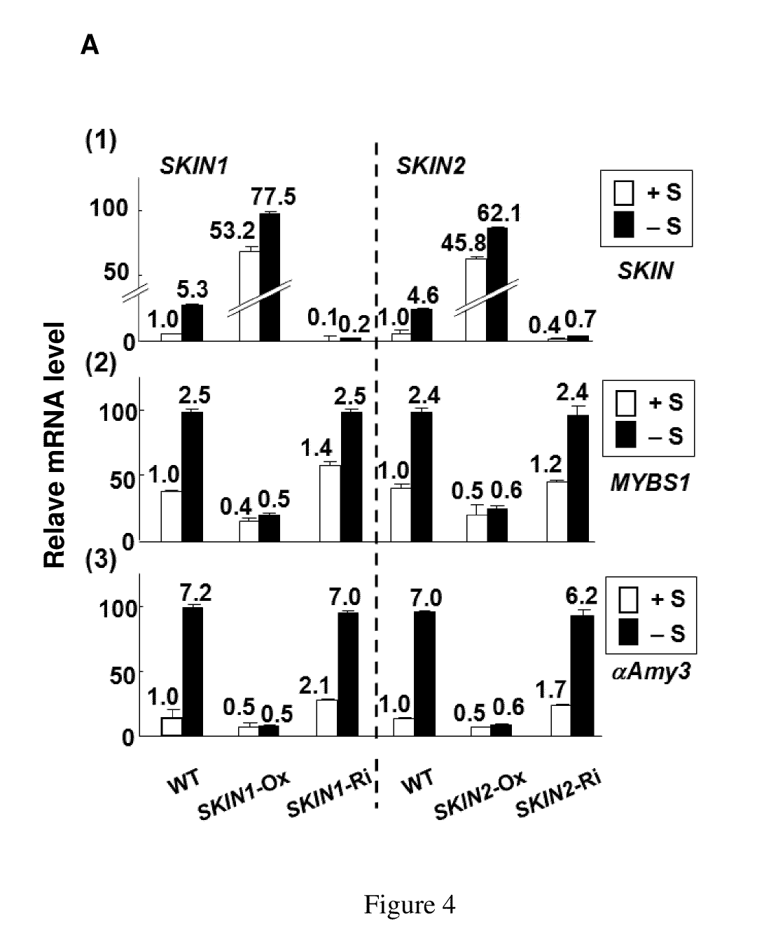

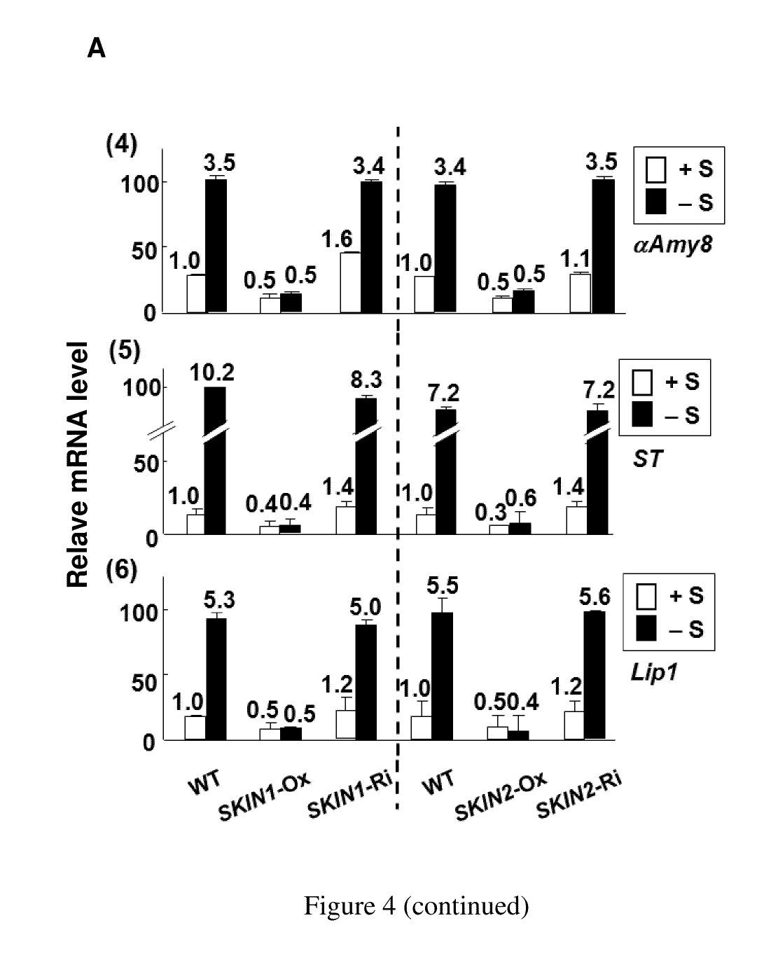

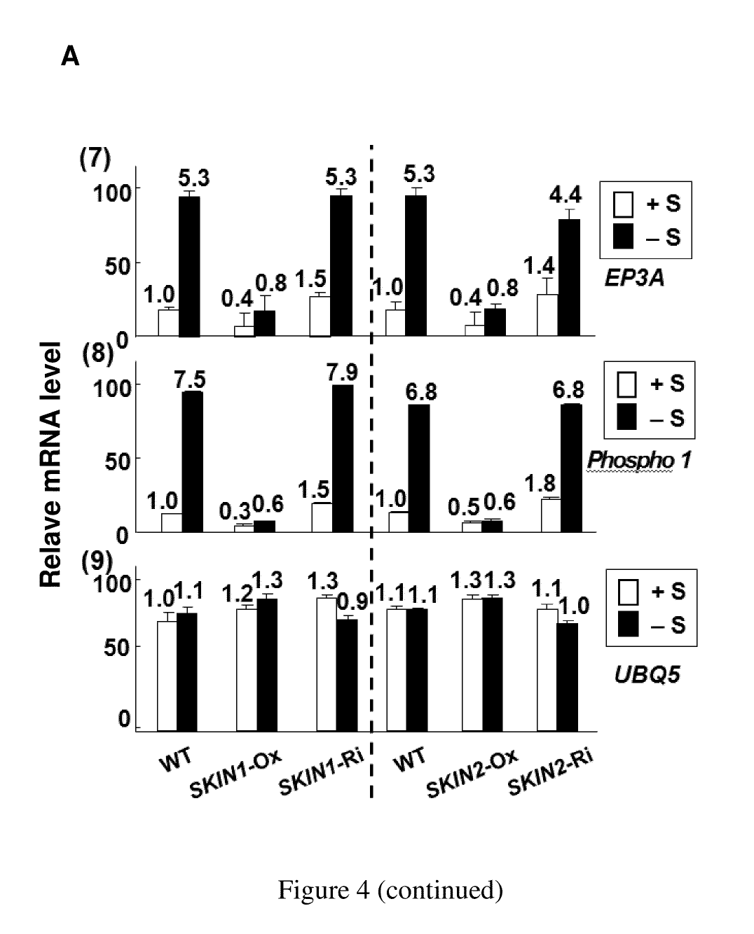

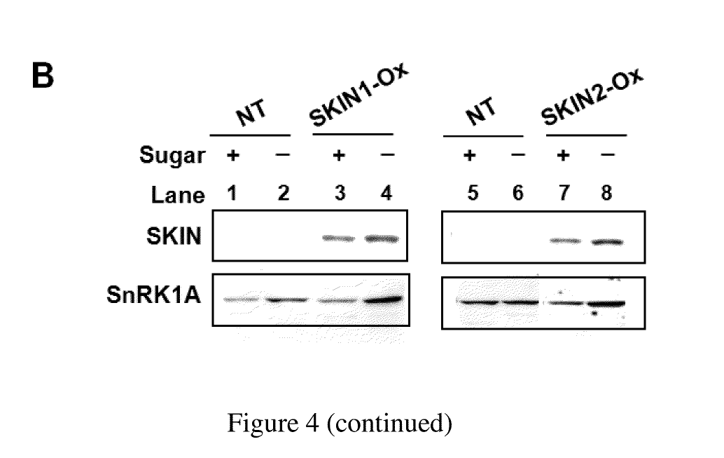

The role of SKINs in the regulation of the SnRK1A-dependent sugar starvation signaling pathway was further explored in transgenic rice carrying constructs Ubi:SKIN and Ubi:SKIN(Ri). In two-day-old transgenic rice seedlings, the accumulation of endogenous SKIN mRNAs in the wild type was up-regulated under −S conditions and decreased in the SKIN-silencing (SKIN-Ri) line under both +S and −S conditions, while the accumulation of recombinant SKIN increased significantly in the SKIN-overexpressing (SKIN-Ox) line under +S and −S conditions (FIG. 4A, panel 1). The expression of hallmarks of the SnRK1A-dependent sugar starvation signaling pathway, including MYBS1, αAmy3 and αAmy8, were all induced in the wild type under −S condition and reduced significantly in the SKIN-Ox line under both +S and −S conditions (FIG. 4A, panels 2-4).

Previously, we showed that the expression of hydrolases and transporters for mobilization of various nutrients stored in the endosperm is coordinately turned on by any nutrient starvation signals at the onset of germination (Hong et al., 2012). To determine whether the SnRK1A-dependent pathway also regulate these genes, we randomly selected four representative genes responsible for carbon, nitrogen, and phosphate nutrient mobilization for further analysis. These included the sugar transporter (ST), GDSL-motif lipase (Lip1), cysteine protease (EP3A), and phosphatase-like protein (Phospho1). The transcription of these four genes is normally low but activated by nutrient starvation (Hong et al., 2012). Here we showed that the accumulation of mRNAs of the four genes was also activated under −S condition and suppressed in the SKIN-Ox line (FIG. 4A, panels 5-8). The accumulation of all tested genes was slightly increased in SKIN-Ri lines under +S but not under −S condition, likely due to the functional redundancy of SKIN1 and SKIN2 under the experimental conditions. The expression of a rice ubiquitin gene, UbiQ5, used as a control was unaltered in SKIN-Ox and SKIN-Ri lines (FIG. 4A, panel 9).

The accumulation of endogenous SnRK1A was slightly higher under −S condition, and the pattern was unaltered by overexpression of SKINs in transgenic rice, except the recombinant SnRK1A slightly increased the level of total SnRK1A (FIG. 4B), indicating that the suppression of the SnRK1A-dependent signaling pathway was not due to the reduction of SnRK1A protein accumulation.

SKINs Repress Seedling Growth by Inhibiting Starch and Nutrient Mobilization from the Endosperm

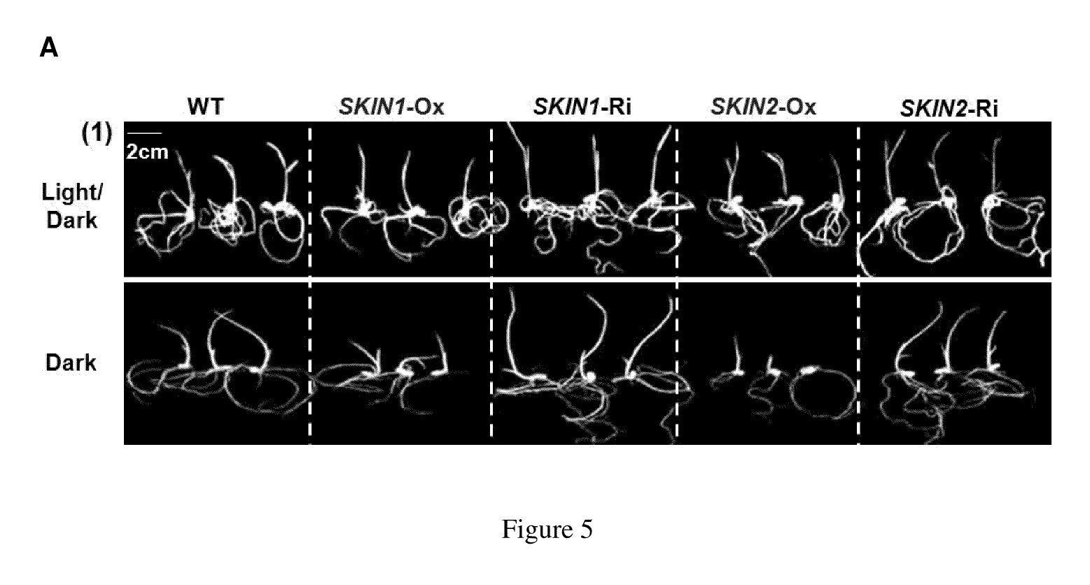

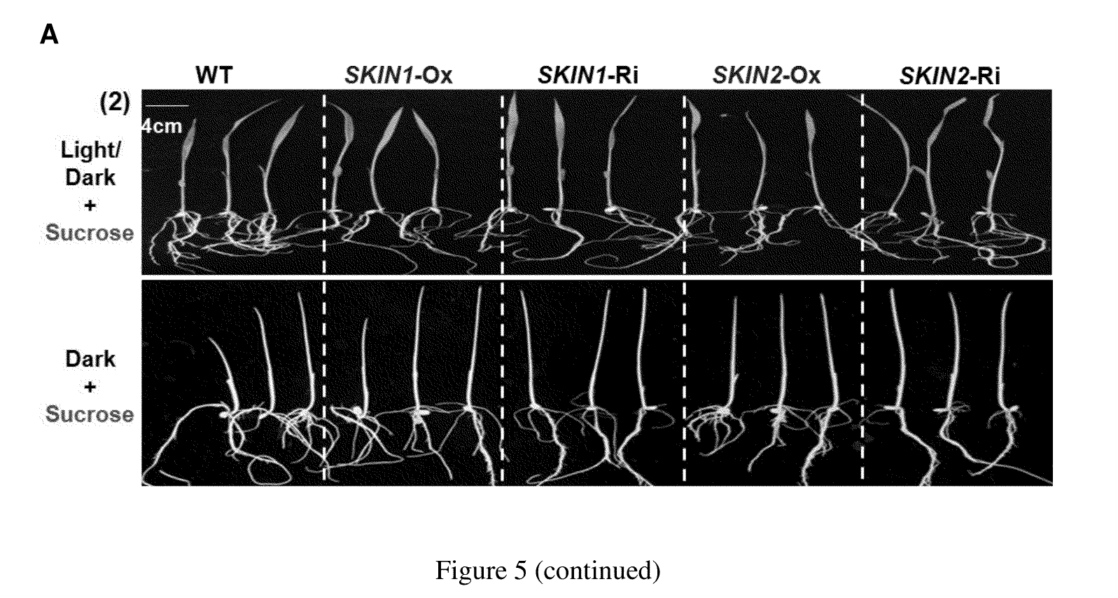

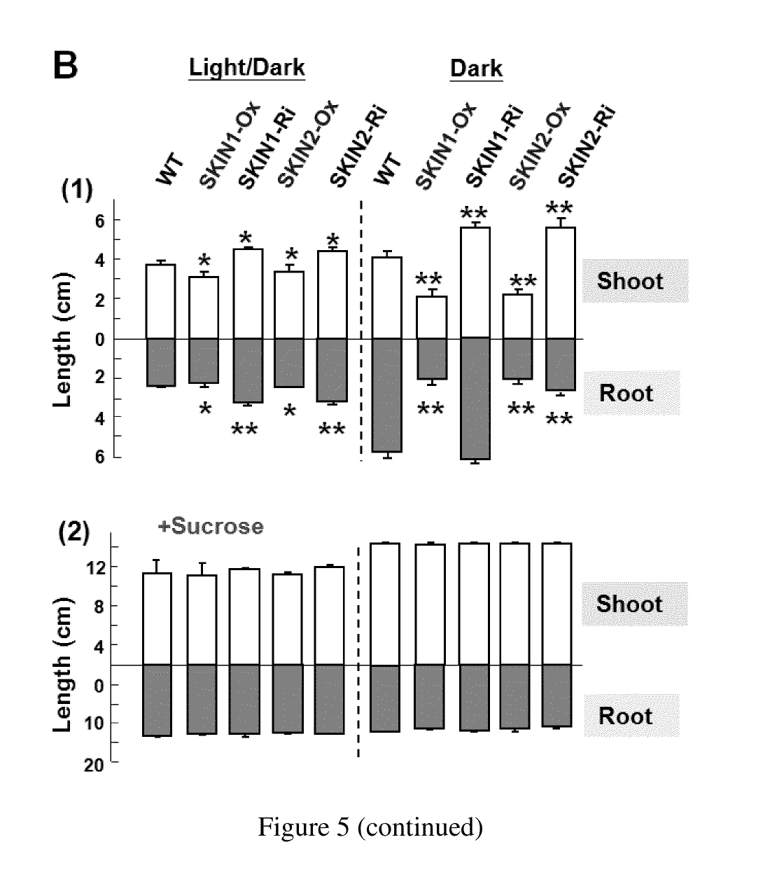

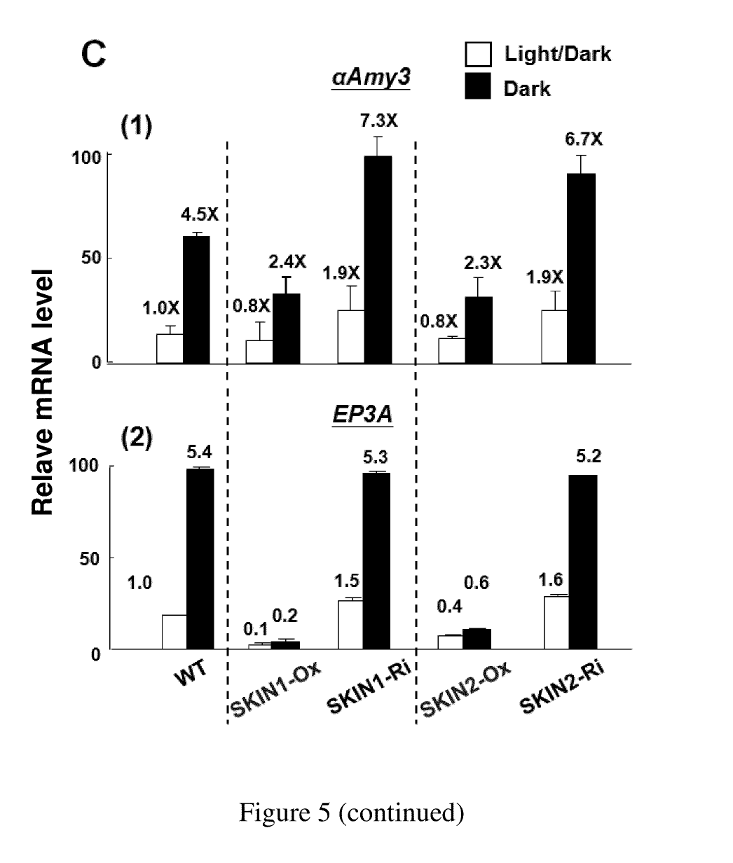

Previously, we showed that germination and seedling growth are retarded in SnRK1A knockout (snf1a) and knockdown (SnRK1-Ri) mutants (Lu et al., 2007). Since SKINs repress the SnRK1A-dependent nutrient starvation signaling pathway in transgenic rice (FIG. 4), the physiological function of SKINs in plant growth was further investigated. SKIN-Ox and SKIN-Ri transgenic lines were grown under the light/dark cycle or continuous dark conditions for 6 days. The growth of shoots and roots under the light/dark cycle were hampered in SKIN-overexpressing (SKIN-Ox) lines but enhanced in SKIN1-silencing (SKIN1-Ri) lines as compared with the wild type, and the difference was more evident under continuous darkness (FIG. 5A, panel 1). Quantitative analyses showed that lengths of both shoots and roots in seedlings were shorter in SKIN-Ox lines and longer in SKIN-Ri lines under the light/dark cycle; this difference was more evident under continuous darkness (FIG. 5B, panel 1). No difference in shoot and root growth was detected regardless of the growth condition if SKIN-Ox and SKIN-Ri lines were provided with 3% (88 mM) sucrose (FIGS. 5A and 5B, panel 2), which indicates that sucrose could recover the growth of SKIN-Ox lines.

To confirm that the inhibition of seedling growth by overexpression of SKINs was resulted from the reduced expression of α-amylase that generates the high-demand carbon source from hydrolysis of seed starch, the expression of αAmy3 was examined. The expression of αAmy3 was induced in 3-day-old seedlings in the wild type under continuous darkness, but the induction was reduced in SKIN-Ox lines and enhanced in SKIN-Ri lines under all growth conditions (FIG. 5C, panel 1). Nitrogen is also essential for seedling growth. The expression of EP3A was regulated similarly to αAmy3 by SKINs, except that its expression was not enhanced in SKIN-Ri lines under continuous darkness (FIG. 5C, panel 2).

### SKINs Repress the Production of Sugars Necessary for Seedling Growth Under Hypoxia

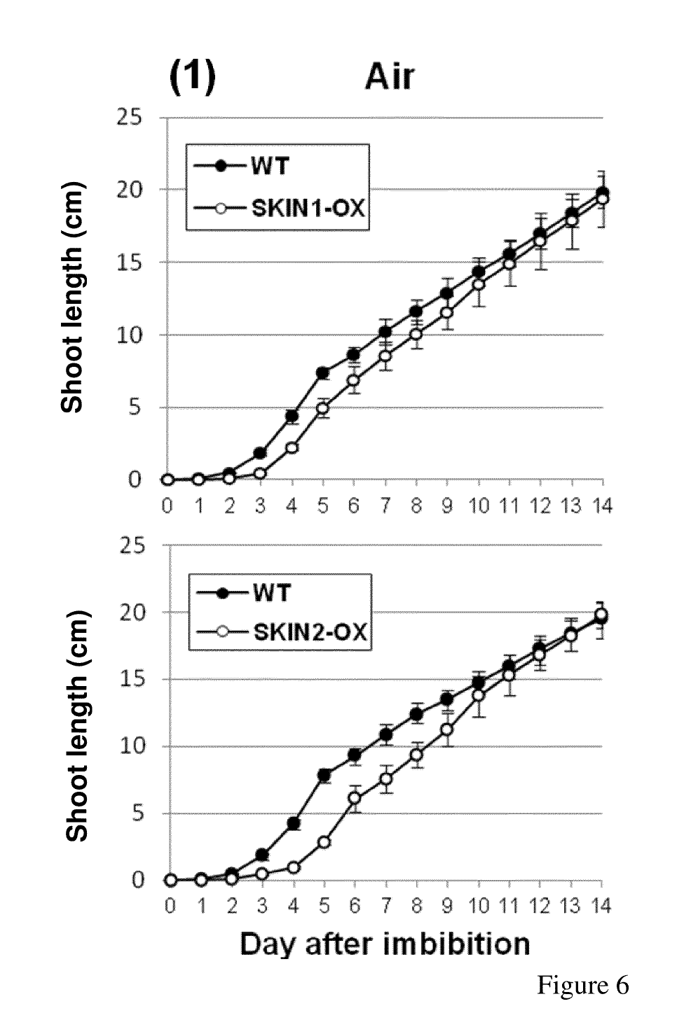

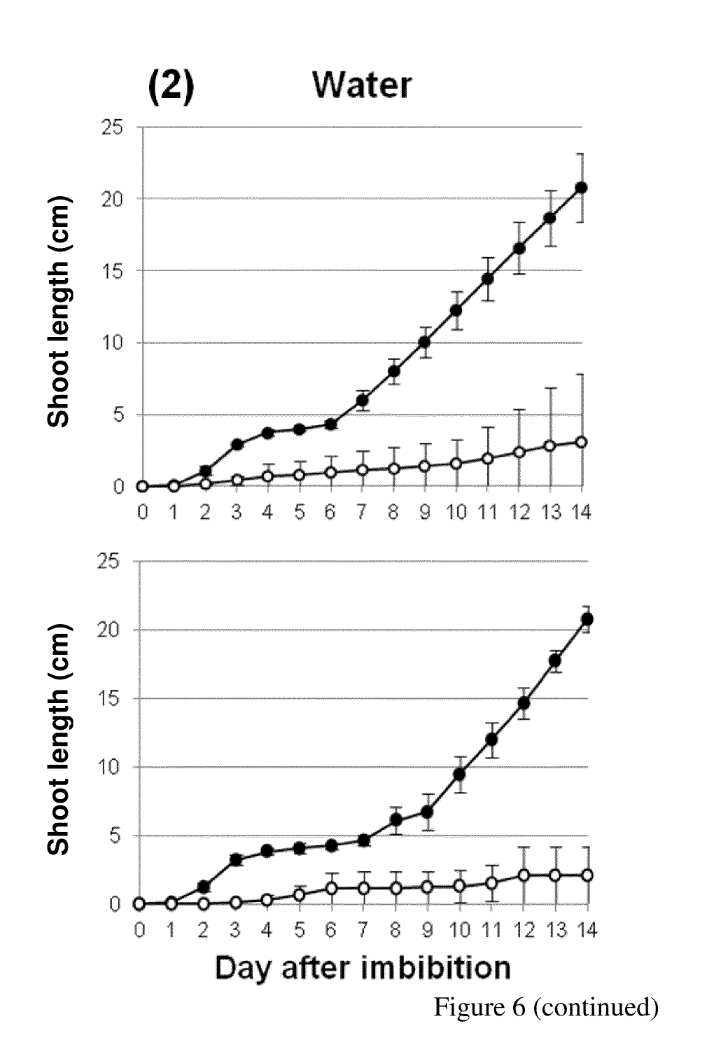

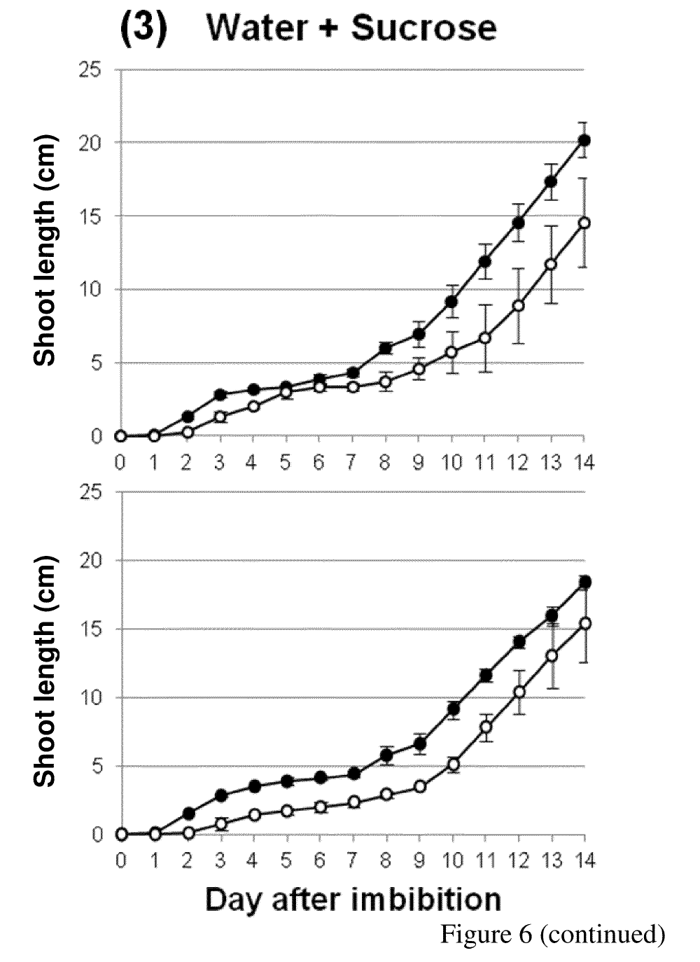

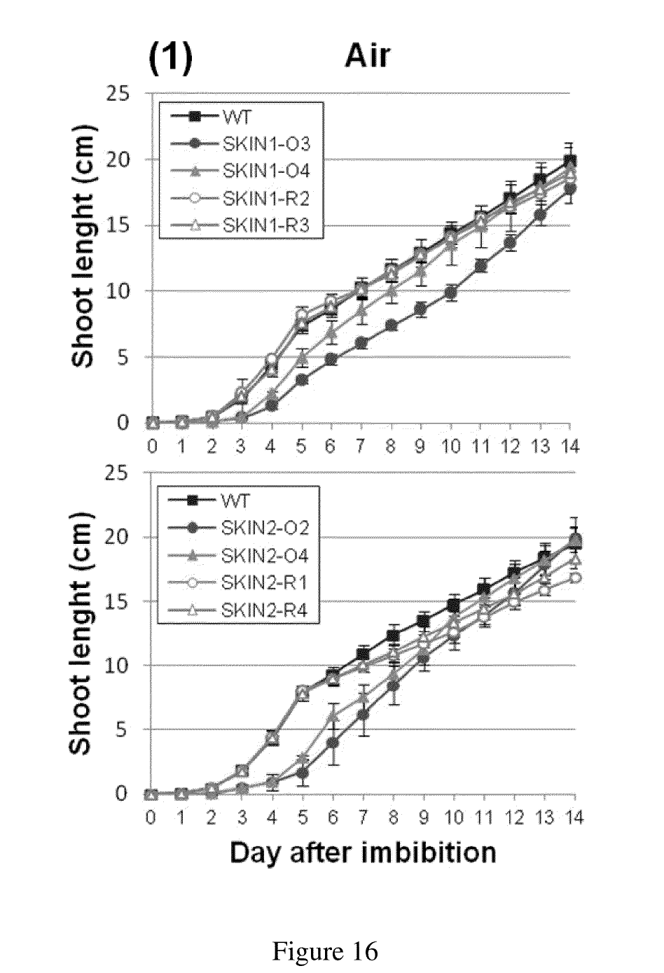

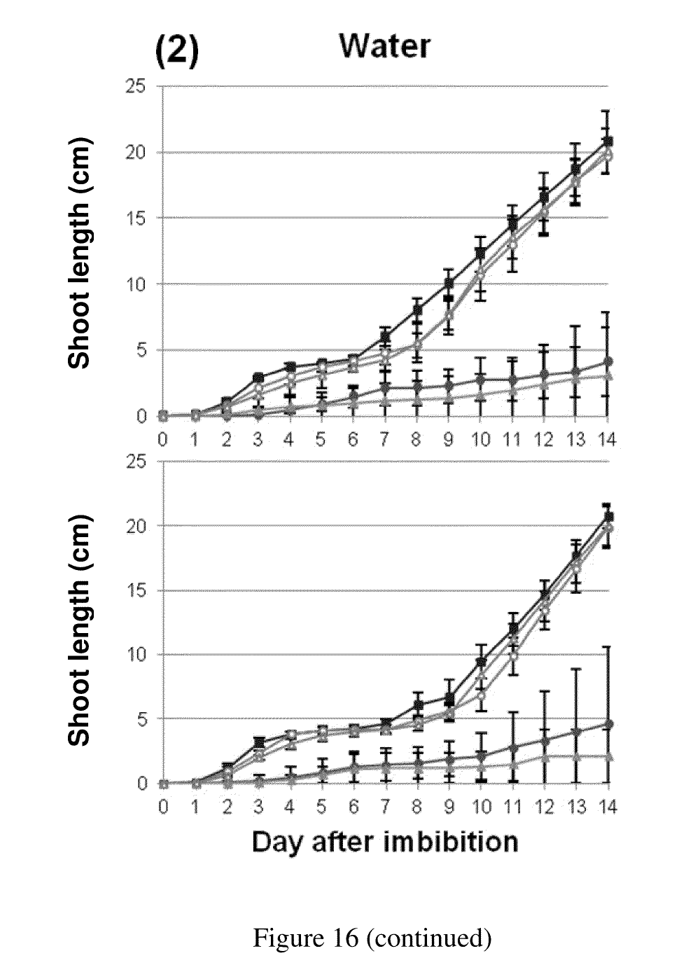

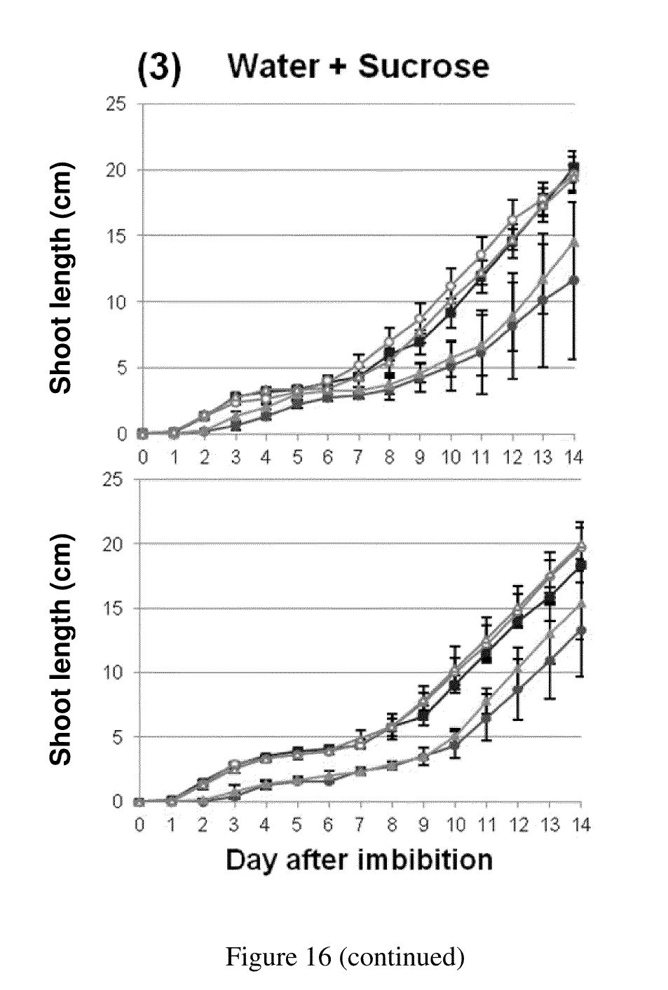

Previously, we showed that SnRK1A acts as an important regulator for germination and seedling growth in rice under hypoxic conditions (Lee et al., 2009). Consequently, the role of SKINs in regulating the hypoxic stress response was also investigated. As shown in FIG. 6 and FIG. 16, in air, shoot elongation of SKIN-Ox lines was slightly slower than the wild type (panel 1), but under water, shoot elongation was severely arrested (panel 2). Under water, the retarded shoot elongation was significantly recovered by sucrose (panel 3). The growth of SKIN-Ri lines was similar to the wild type. These results further confirm that SKINs suppress the SnRK1A-dependent pathway, leading to impaired sugar production from starch hydrolysis in seeds during the post-germination seedling growth under hypoxia.

### SKINs and SnRK1A Interact Primarily in the Cytoplasm

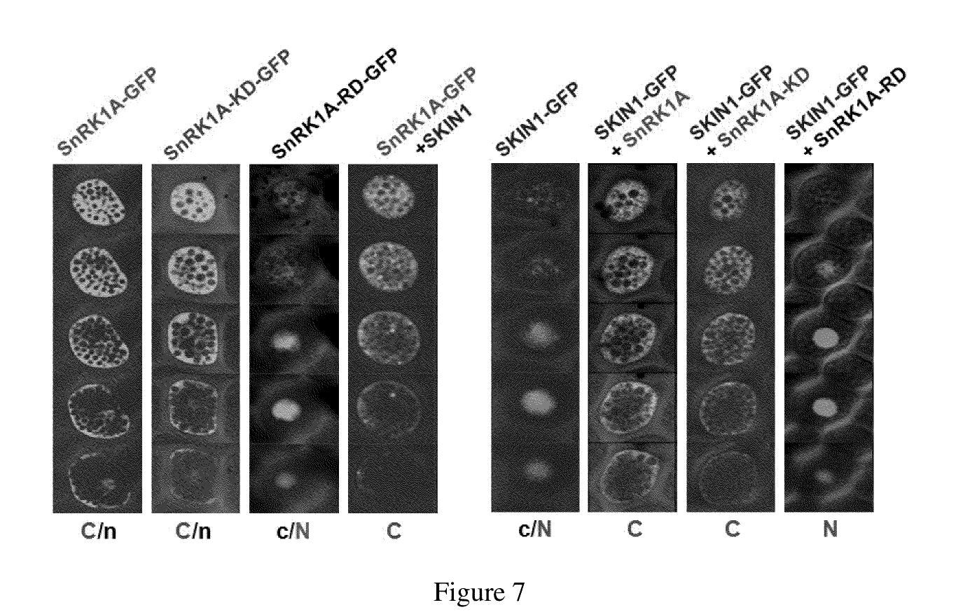

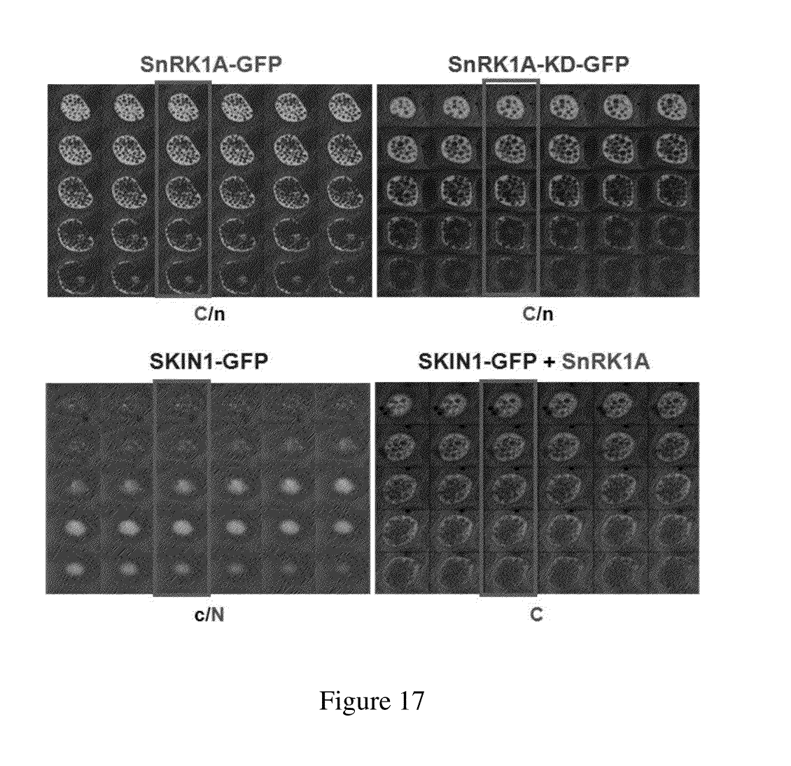

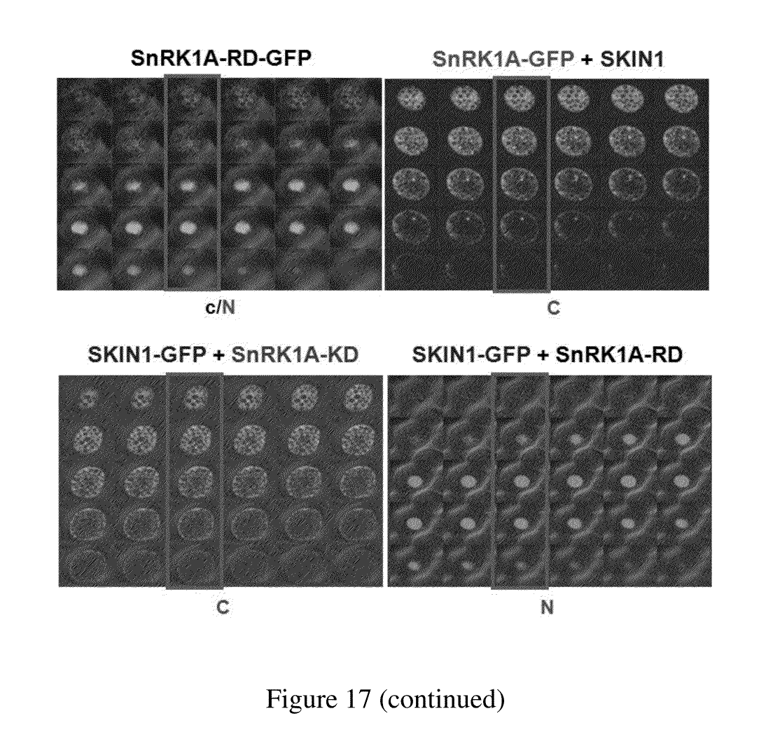

The subcellular localization of SKIN and SnRK1A was determined. As SKINs interact with the KD of SnRK1A, the full-length, KD and RD of SnRK1A were fused to the green fluorescence protein (GFP) and expressed under the control of the Ubi promoter in a barley aleurone cell transient expression system (Hong et al., 2012). As shown in FIG. 7 and FIG. 17, SnRK1A-GFP and SnRK1A-KD-GFP were largely localized in the cytoplasm and minor in the nucleus and SnRK1A-RD-GFP mainly in the nucleus, whereas SKIN1-GFP was predominantly localized in the nucleus and minor in the cytoplasm. Co-expression of SnRK1A-GFP with SKIN1 excluded all SnRK1A-GFP from the nucleus. Co-expression of SKIN1-GFP with SnRK1A or SnRK1A-KD sequestered all SKIN1-GFP in the cytoplasm, whereas with SnRK1A-RD maintained the nuclear localization of SKIN1-GFP. These studies demonstrate that SKIN1 interacts with SnRK1A through SnRK1A-KD, which is consistent with result using the plant two-hybrid assay (FIG. 2C), and the interaction retained SKINs and SnRK1A in the cytoplasm.

### SKINs Antagonize the Function of SnRK1A in Both the Cytoplasm and Nucleus

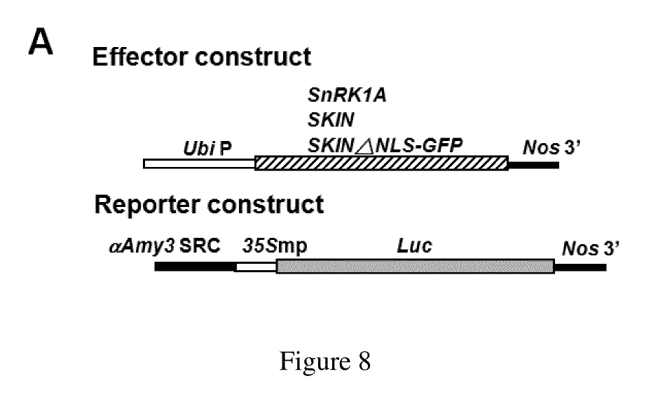

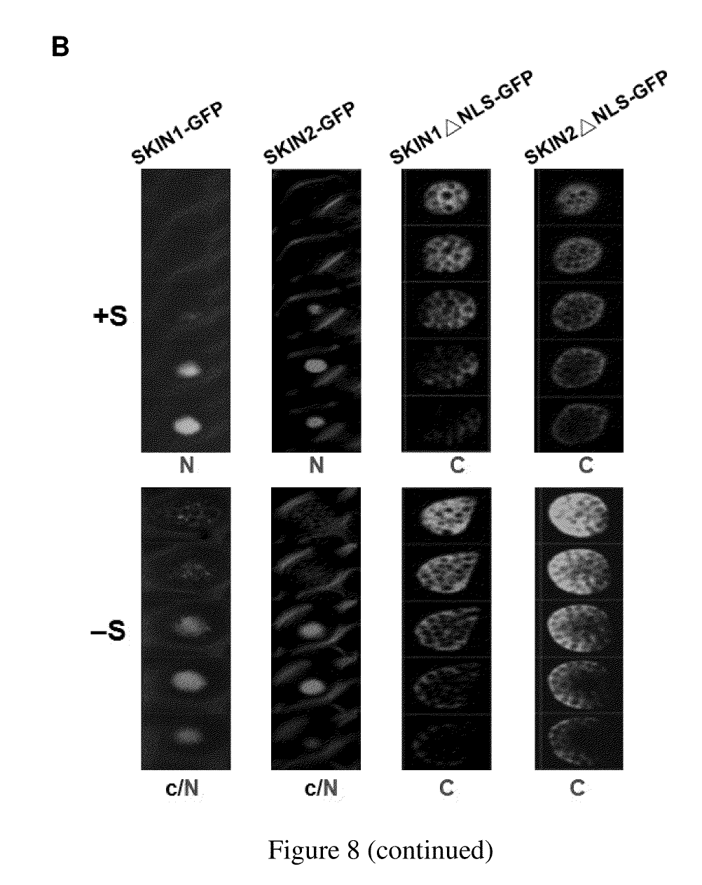

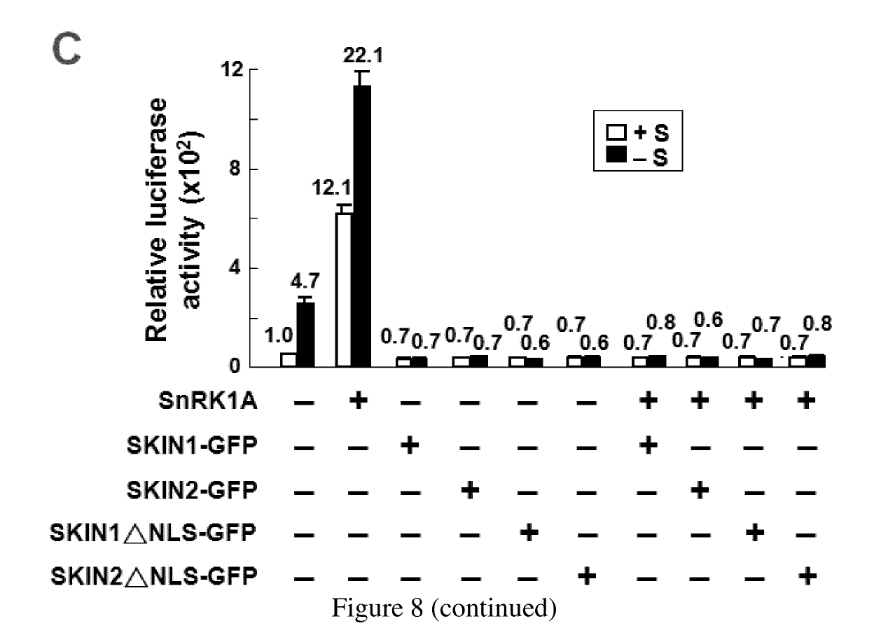

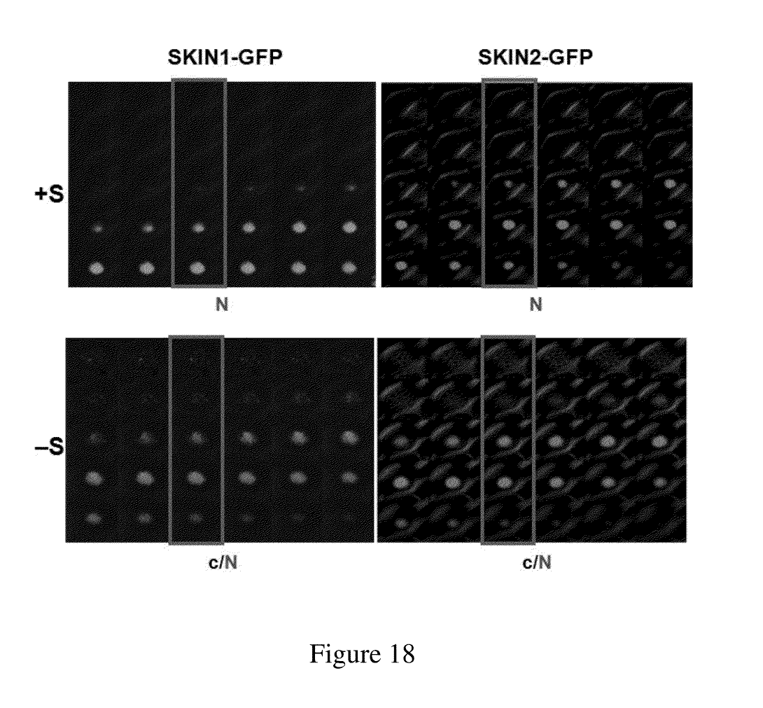

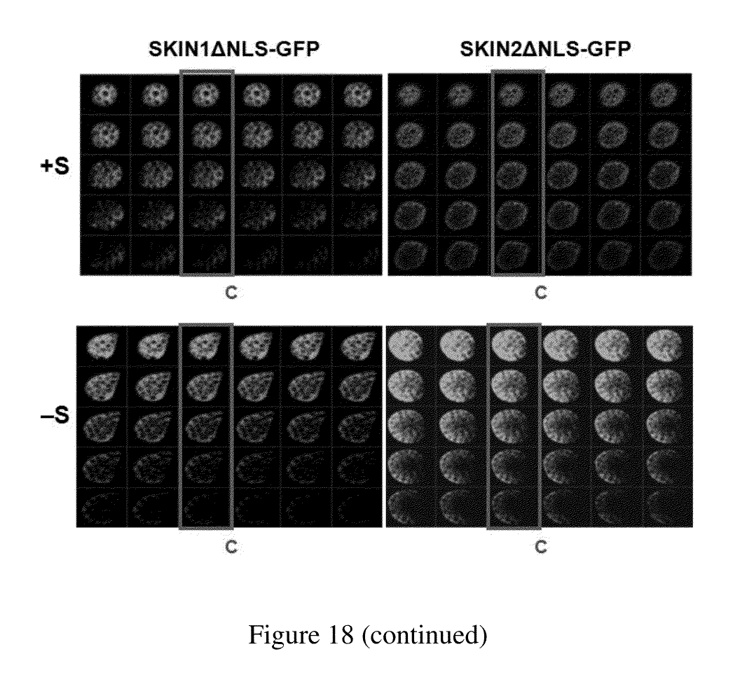

Since SnRK1A and SKINs are present in both the cytoplasm and nucleus (FIG. 7), we determined whether SKINs could antagonize the function of SnRK1A in both the nucleus and cytoplasm. The putative NLS in SKINs was deleted (SKINΔNLS) and fused to GFP (FIG. 8A). SKIN-GFP was mainly localized in the nucleus whereas SKINΔNLS-GFP was exclusively localized in the cytoplasm under both +S and −S conditions (FIG. 8B and FIG. 18), which indicates that the predicated NLS was functional. Co-expression of SKIN-GFP with or without NLS with SnRK1A repressed αAmy3 SRC promoter to a level similar to overexpression of SKIN-GFP alone (FIG. 8C). It also indicates that SKIN in the cytoplasm could still trap SnRK1A to the cytoplasm, which prevents the up-regulation of MYBS1 expression that is needed for αAmy3 SRC activity.

### The Expression of SKIN is Induced by Various Abiotic Stresses and ABA, and SKINs Promote the ABA Sensitivity

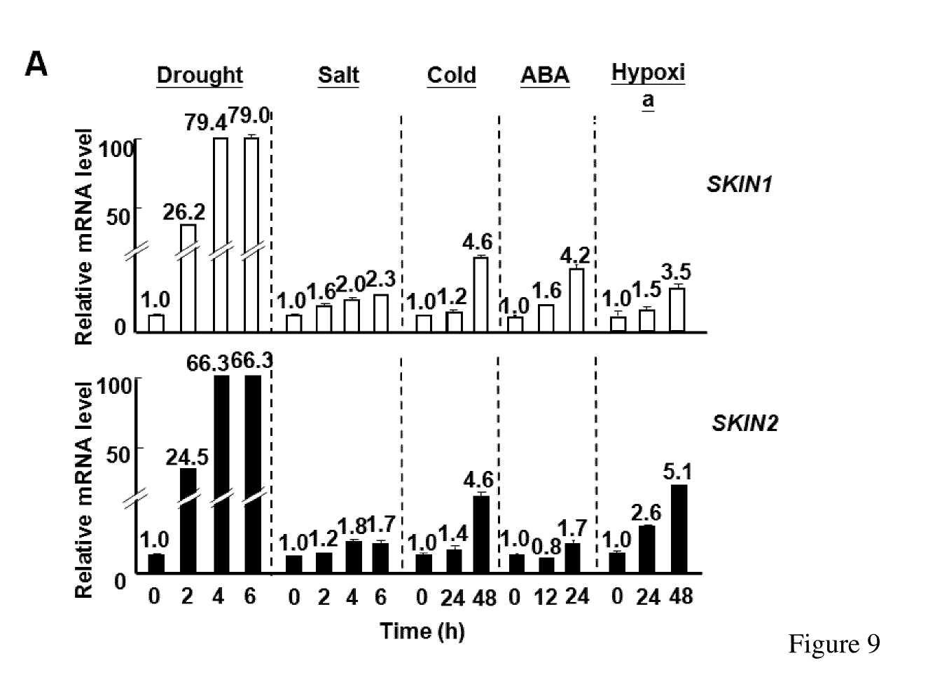

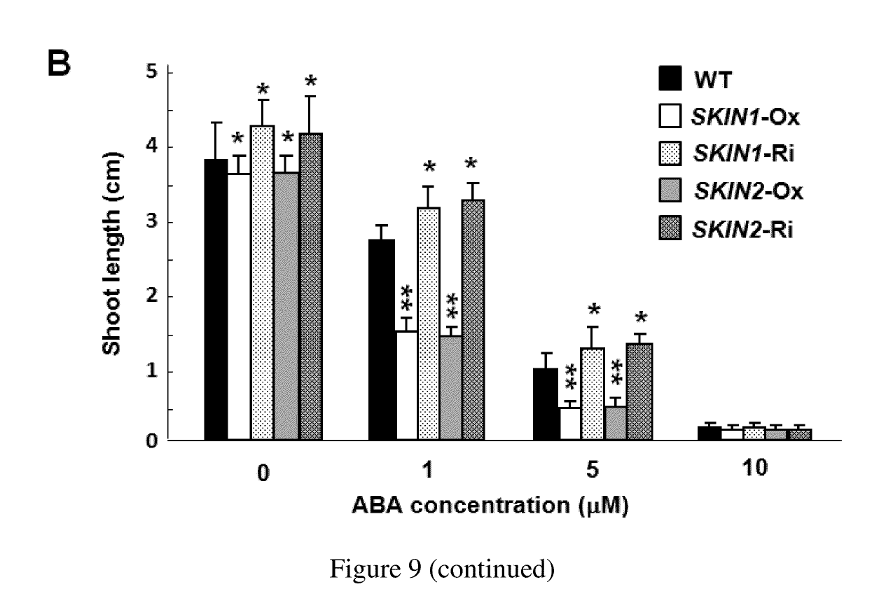

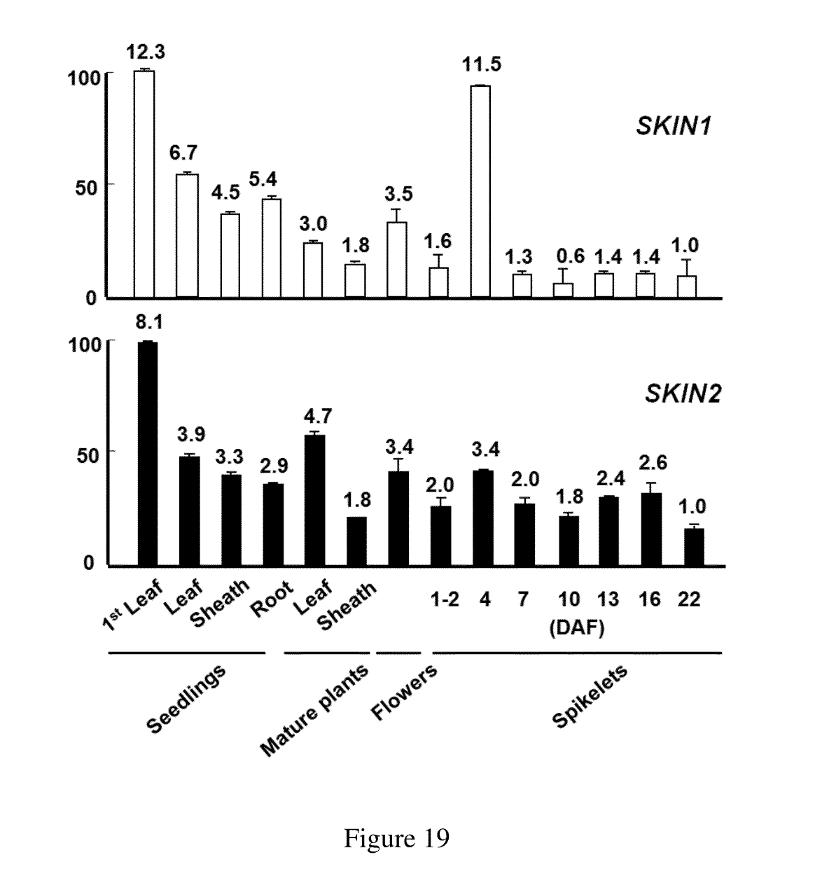

Expression of both SKINs could be detected in all tissues in seedlings, mature plants, flowers, and immature panicles, and is particularly highly induced in the first leave of seedlings and at day 4 after flowering (FIG. 19). We also determined whether the expression of SKINs is regulated by abiotic stresses. Rice seedlings were subjected to drought (exposure to dry air), salt (200 mM NaCl), cold (4° C.) and hypoxia treatments. The accumulation of SKIN1 and SKIN2 mRNAs was induced up to 79- and 66-fold, respectively, at 4 h after drought stress, 2.3- and 1.7 fold, respectively, 6 h after salt stress, 4.6-fold for both SKIN1 and SKIN2 48 h after cold stress, 4.2- and 1.7-fold, respectively, 24 h after ABA, and 3.5- and 5.1-fold, respectively, 48 h after hypoxia treatment (FIG. 9A).

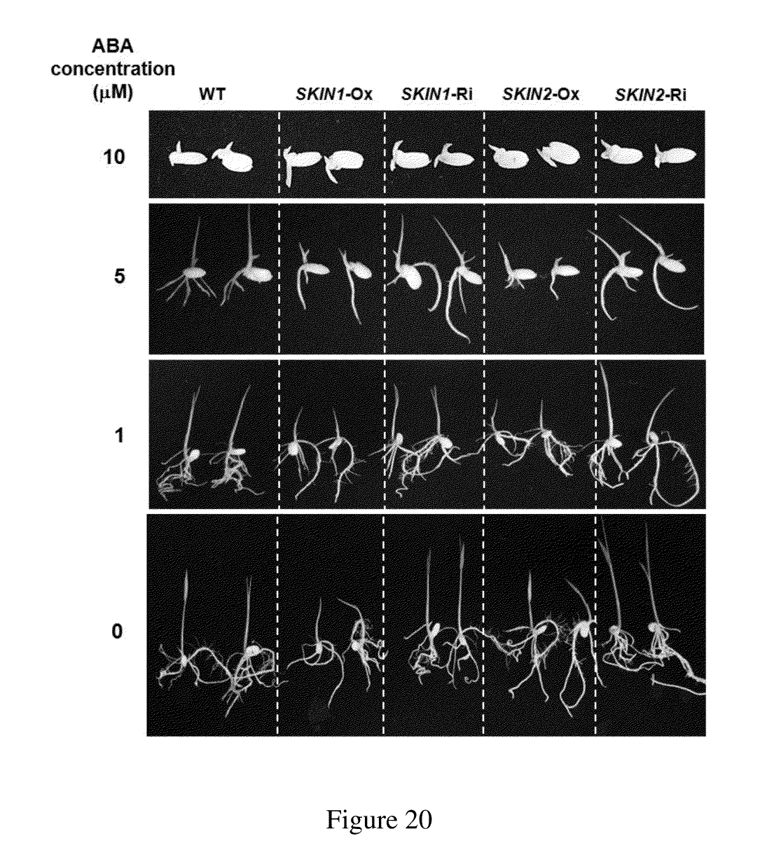

To determine whether SKINs are important for ABA response/signaling, SKIN-Ox and SKIN-Ri lines were germinated in water containing various concentrations of ABA. The degree of inhibition on growth of wild type and all transgenic lines increased with ABA concentrations from 1 to 10 μM; however, the growth of SKIN-Ri lines was less, and that of SKIN-Ox lines was more severely, inhibited by 1 and 5 μM of ABA than the wild type (FIG. 9B and FIG. 20). These results demonstrate that SKINs promote the ABA sensitivity.

### ABA Restricts SKINs, SnRK1A and MYBS1 in the Cytoplasm Under Sugar Starvation

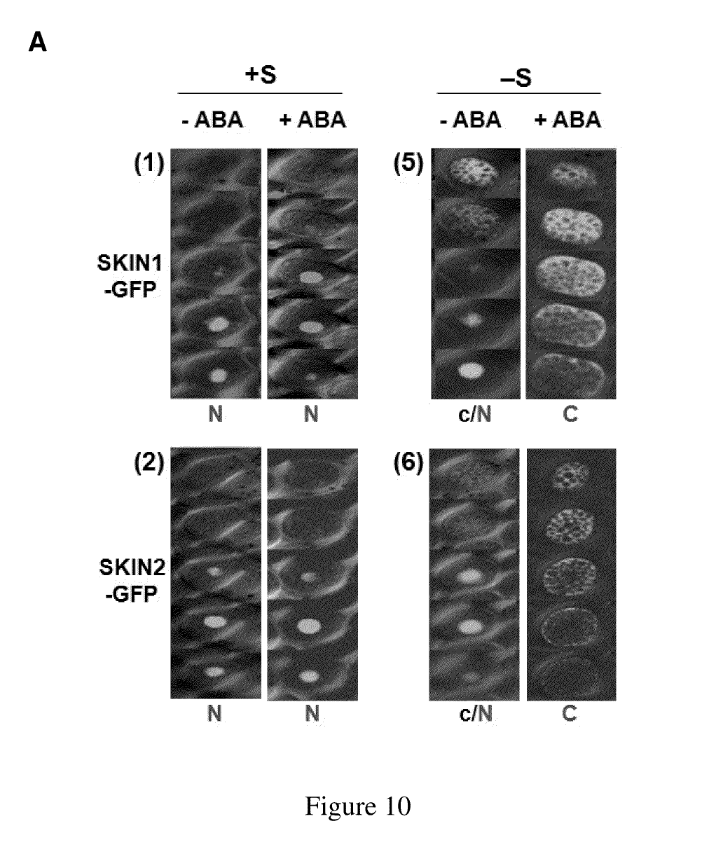

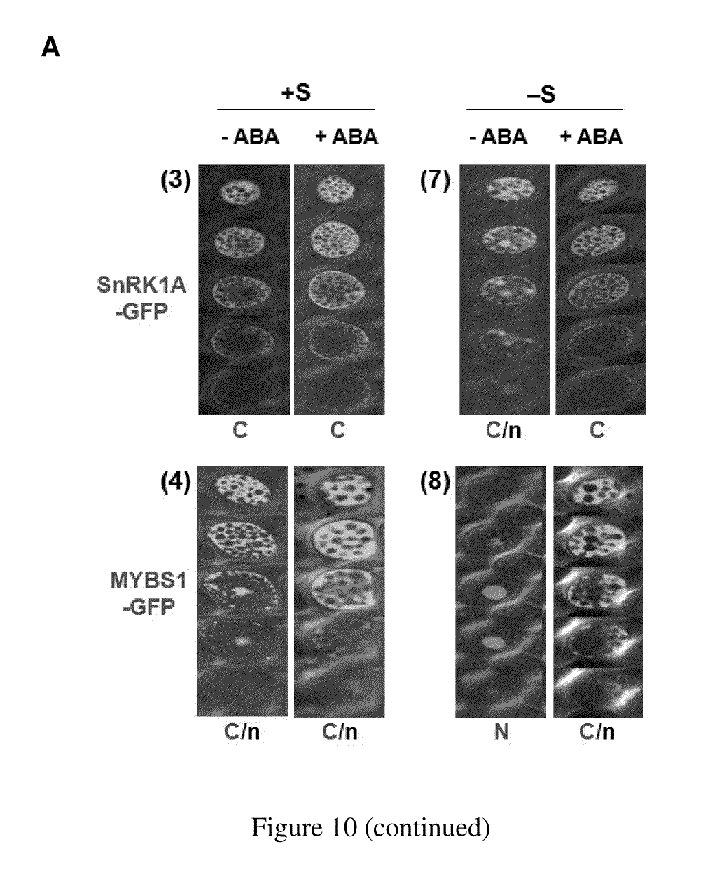

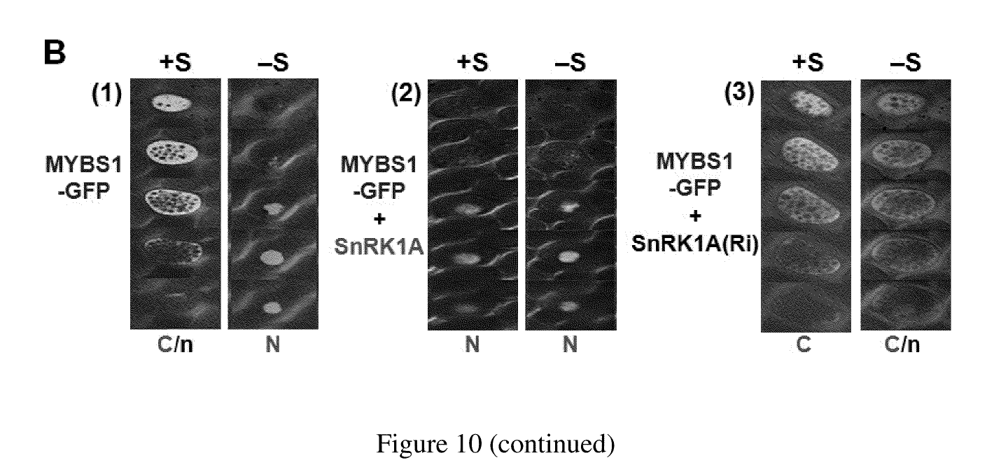

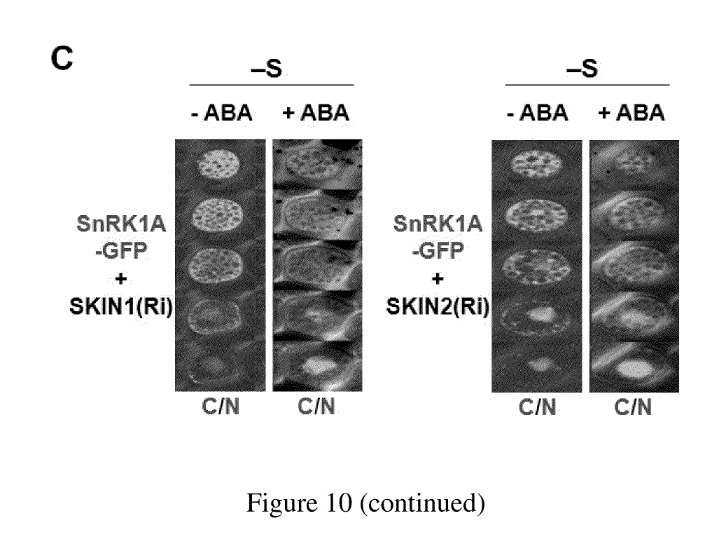

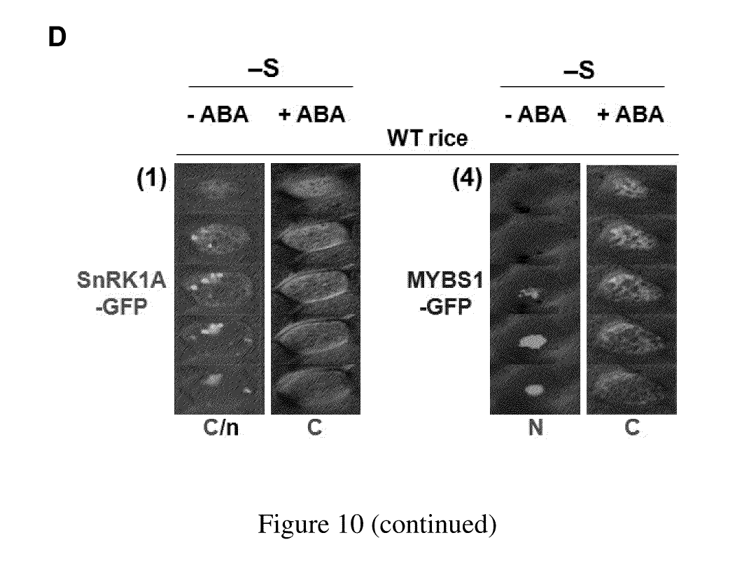

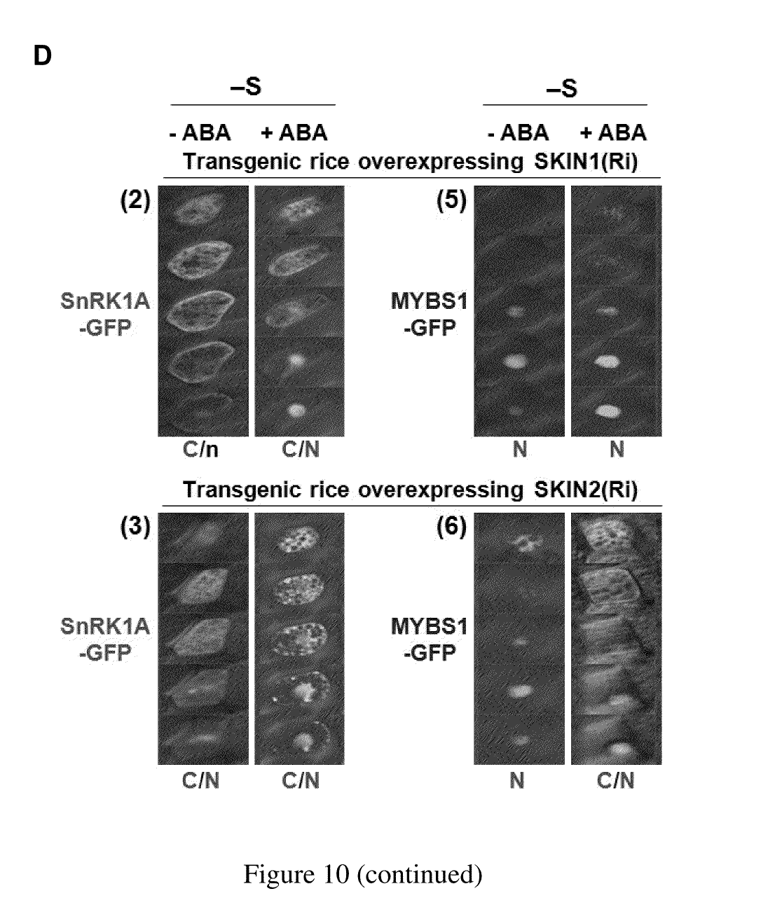

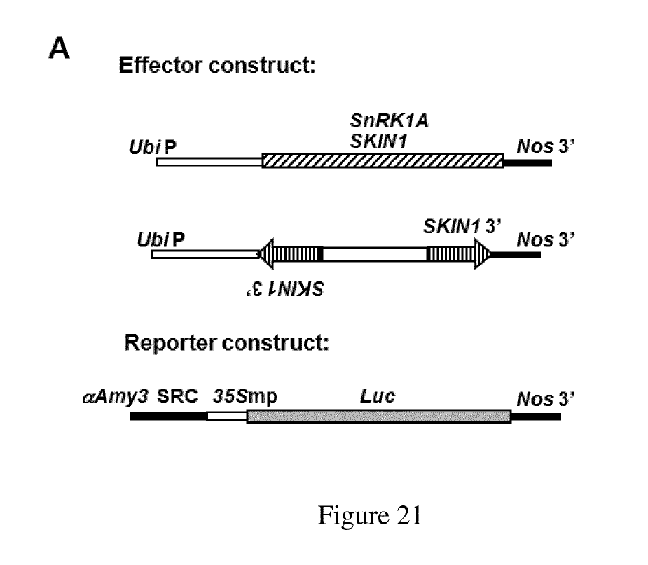

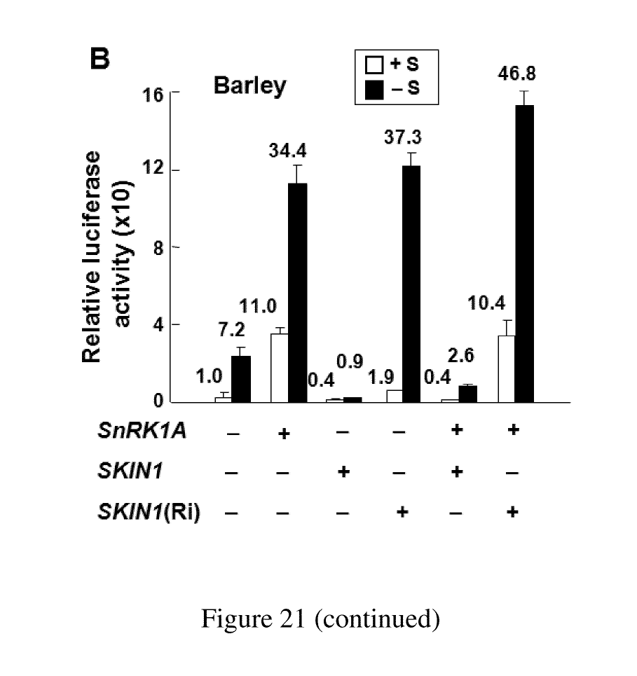

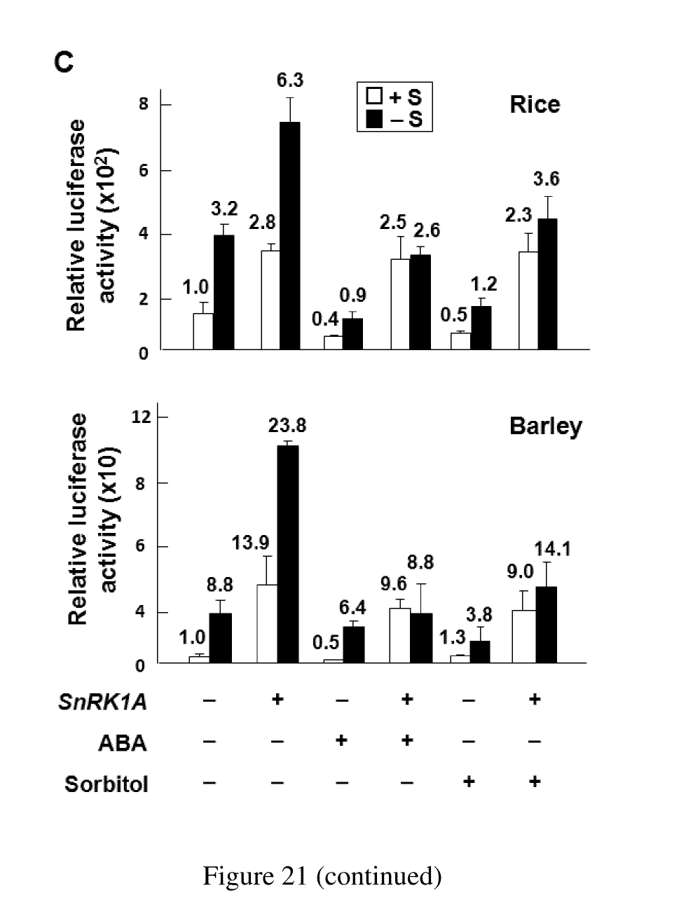

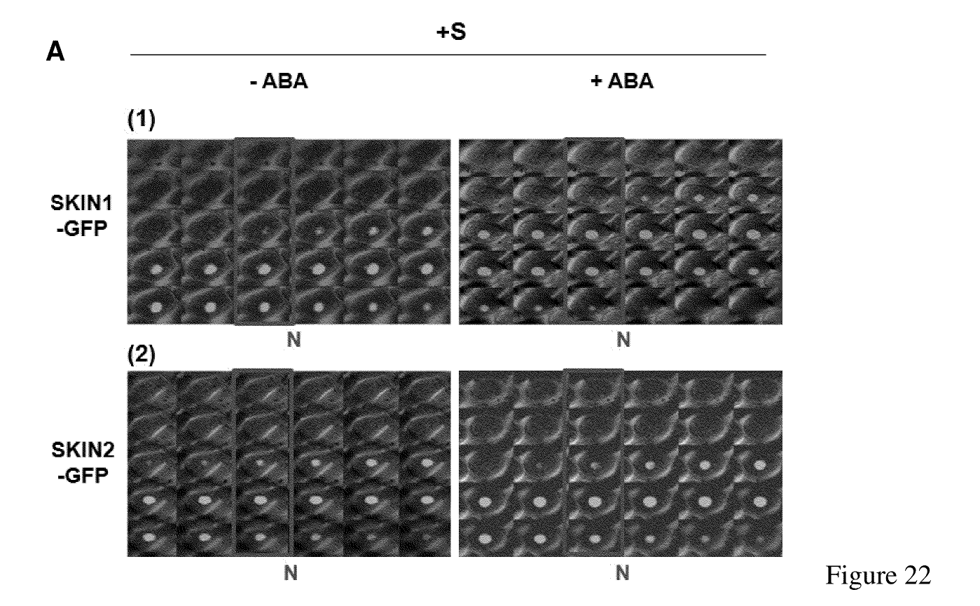

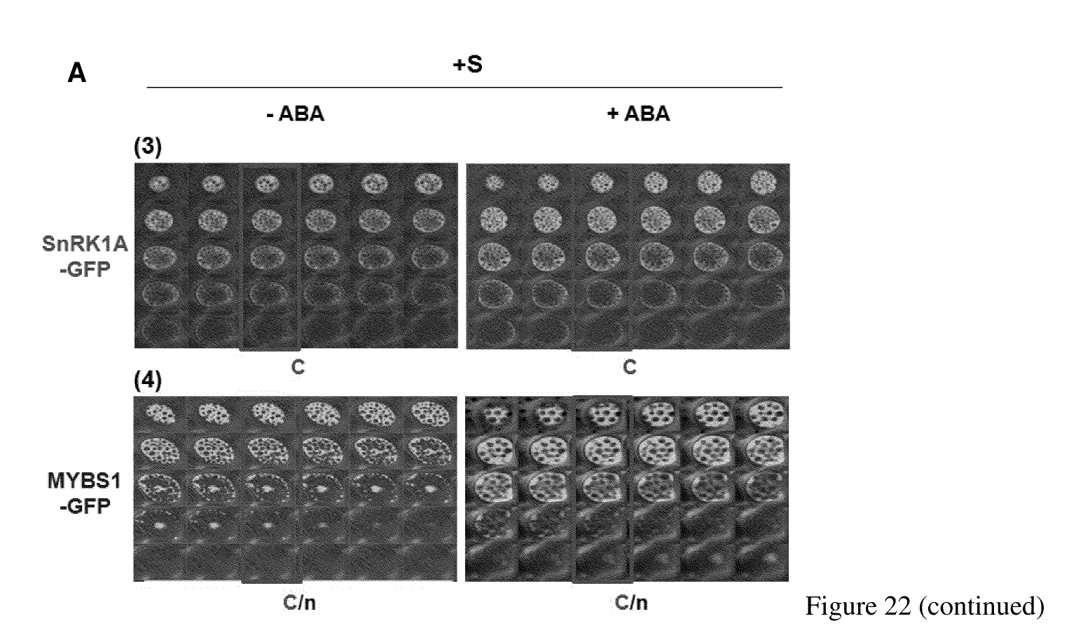

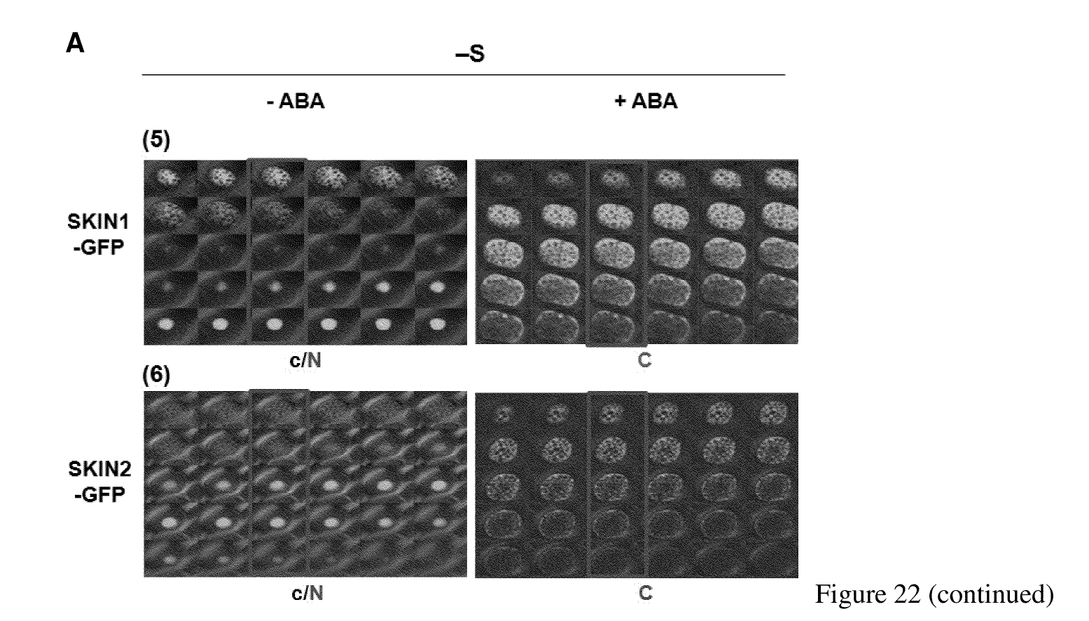

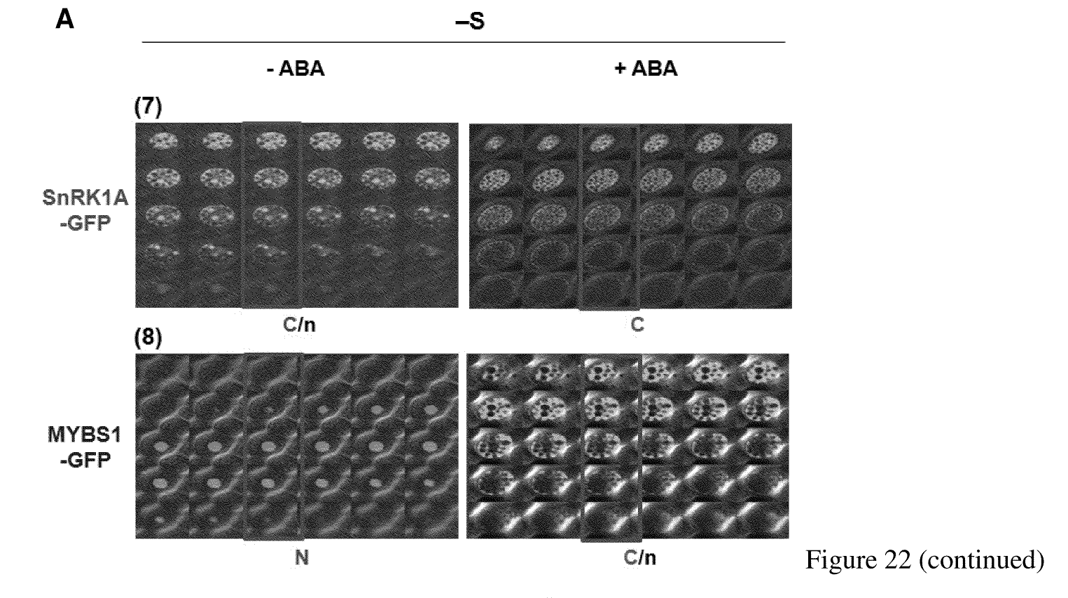

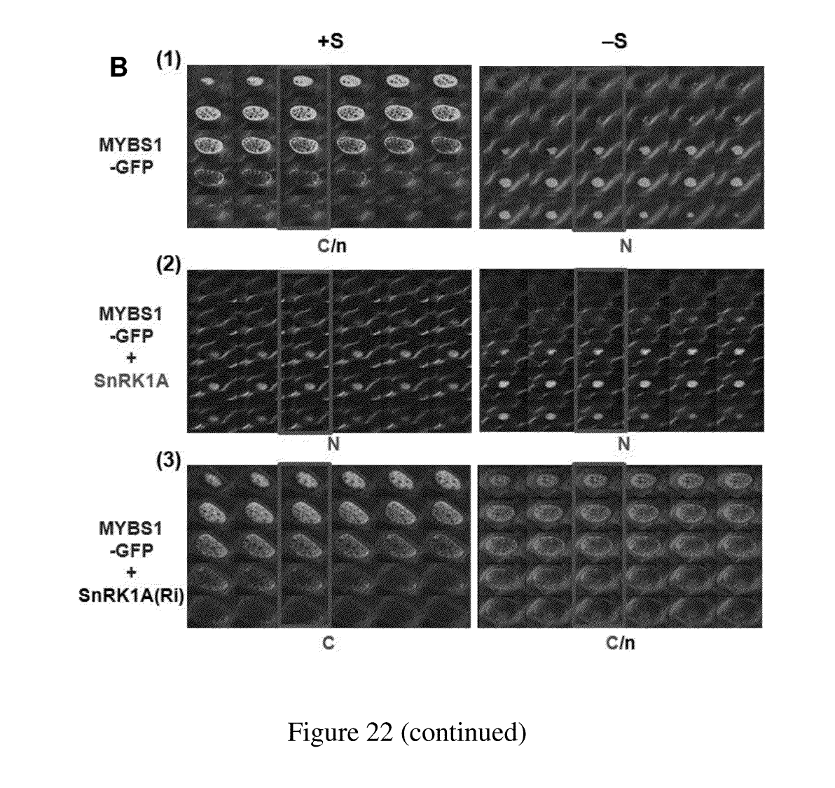

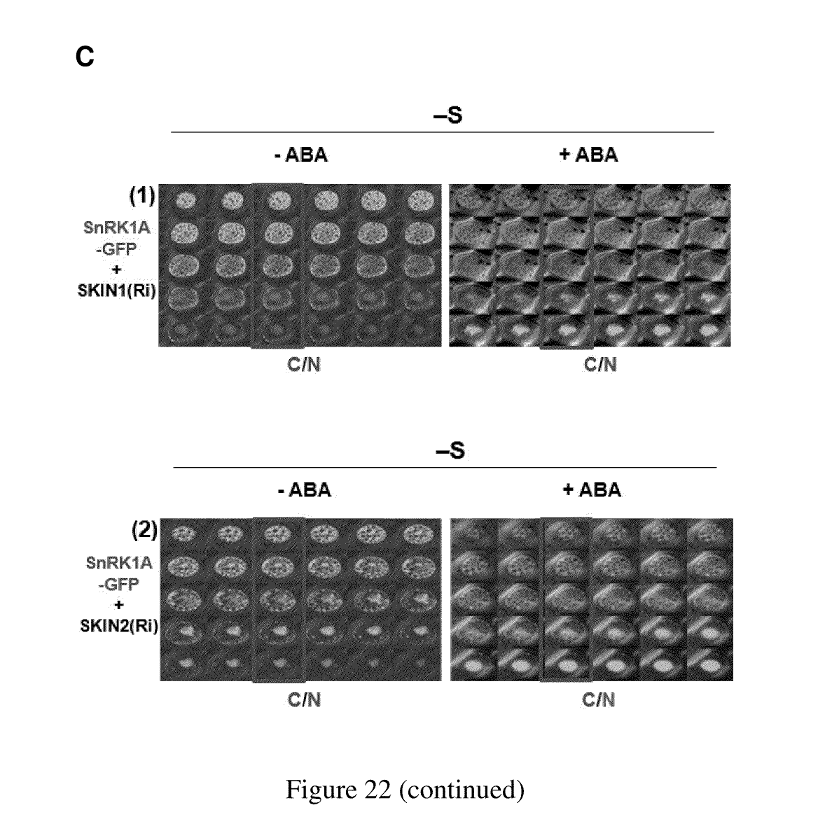

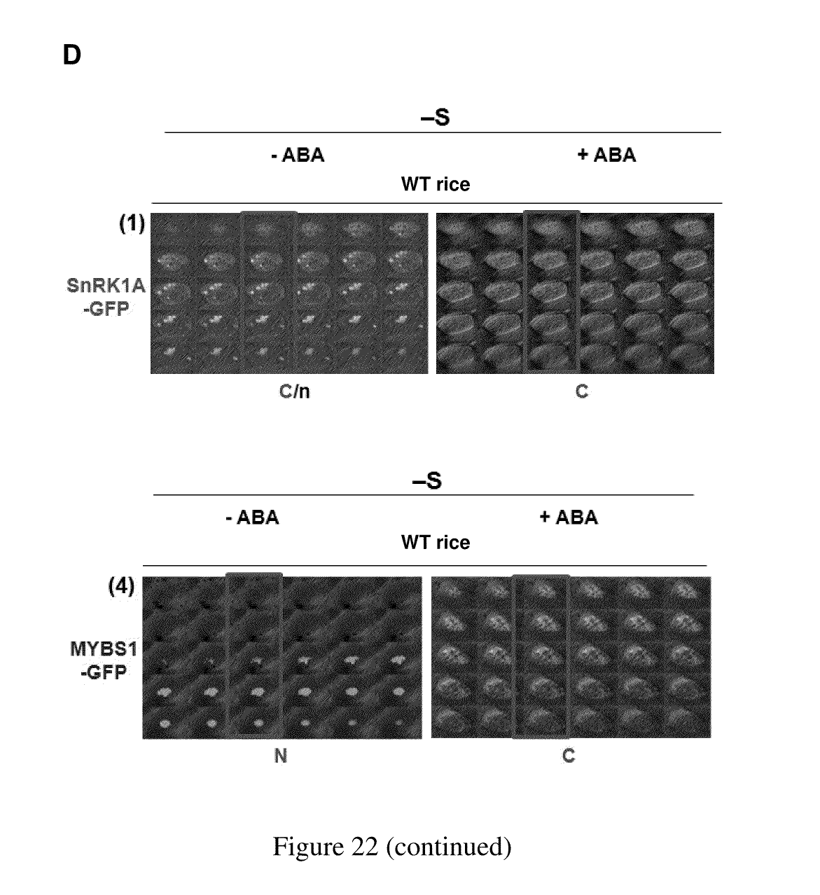

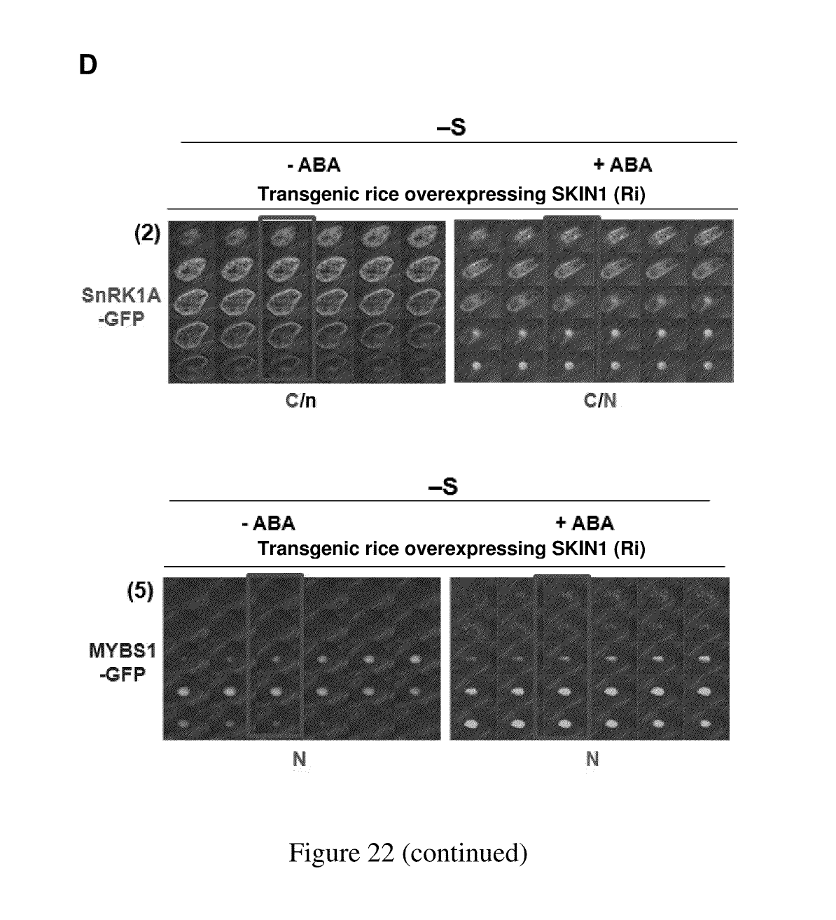

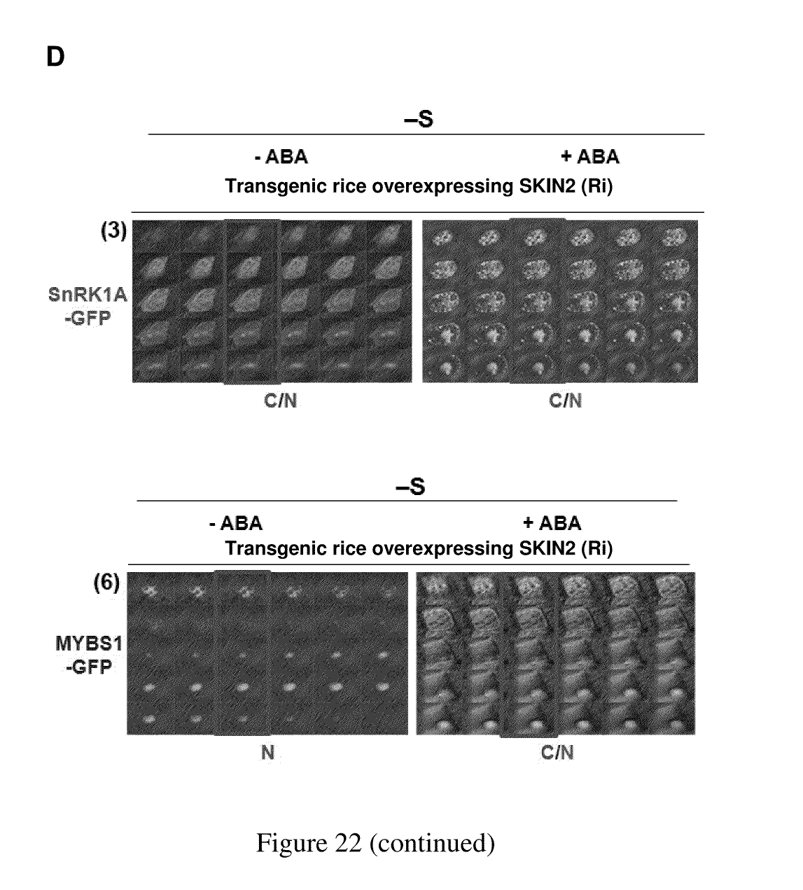

Above studies showed that SKINs are exclusively localized in the nucleus in +S medium but levels are increased in the cytoplasm in −S medium, and they could antagonize the function of SnRK1A in both the nucleus and cytoplasm (FIGS. 7 and 8). Since the expression of SKINs is induced by various abiotic stresses and ABA, it is essential to determine whether SKINs are shuttling between the nucleus and cytoplasm in a stress-dependent manner. ABA and sorbitol, the latter mimic osmotic stress, not only by themselves suppressed, but also antagonized the SnRK1A-activated αAmy3 SRC promoter in both rice embryos and barley aleurones (FIG. 21). ABA also enhanced the interaction between SnRK1A and SKINs in rice embryos (FIG. 2D). Consequently, ABA was used as a stress signal inducer. SKINs, SnRK1A and MYBS1 fused to GFP were transiently expressed in barley aleurones incubated in +S or −S medium with or without ABA. SKIN-GFP and SnRK1A-GFP were exclusively localized in the nucleus and cytoplasm, respectively, in +S medium with or without ABA (FIG. 10A and FIG. 22A, panels 1-3). SKIN-GFP became detectable in the cytoplasm and a considerable amount of SnRK1A in the nucleus in −S medium without ABA; however, both SKIN-GFP and SnRK1A-GFP became exclusively localized in the cytoplasm in −S medium containing ABA (FIG. 10A and FIG. 22A, panels 5-7). Quantitative analyses revealed that, in the absence of ABA, the percentage of SnRK1A-GFP localized in the nucleus was 19.7% and 64.0% in +S and −S medium, respectively, indicating that sugar starvation promotes the nuclear localization of SnRK1A (Table 4). In −S medium, the percentage of SnRK1A-GFP localized in the nucleus was reduced from 64.0% in the absence of ABA to 8.0% in the presence of ABA, indicating that ABA inhibits the nuclear localization of SnRK1A (Table 4).

TABLE 4
ABA inhibits the nuclear localization of SnRK1A.

| | Location of | +S | | −S | |
|---|---|---|---|---|---|
| | SnRK1A-GFP | −ABA | +ABA | −ABA | +ABA |
| Number of cells in different locations (% of total) | 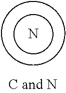 | 13 (19.7%) | 6 (15.8%) | 71 (64.0%) | 7 (8.0%) |
| | 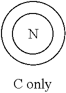 | 53 (80.3%) | 32 (84.2%) | 40 (36.0%) | 81 (92.0%) |
| Total cell number | | 66 | 38 | 111 | 88 |

Barley aleurones were transfected with Ubi:SnRK1A-GFP and incubated in
+S or −S medium with ABA (+ABA) or without ABA (−ABA) for 48 h.
Percentages indicate the number of cells with GFP distribution in the indicated
category divided by the total number of cells examined. C: cytoplasm; N: nucleus.

MYBS1-GFP was mostly localized in the cytoplasm in +S medium and exclusively in the nucleus in −S medium without ABA, which is consistent with our previous study (Hong et al., 2012); however, MYBS1-GFP became exclusively localized in the cytoplasm in −S medium containing ABA (FIG. 10A and FIG. 22A, panels 4 and 8). MYBS1 has been shown to be activated transcriptionally by SnRK1A (Lu et al., 2007). Here, we found that the nuclear import of MYBS1 was also promoted by overexpression of SnRK1A in +S medium and inhibited by silencing of SnRK1A in −S medium (FIG. 10B and FIG. 22B, panels 2 and 3, respectively), indicating that SnRK1A is sufficient and necessary for promoting the nuclear localization of MYBS1. These studies also indicate that the nuclear localization of SnRK1A and MYBS1 are suppressed by ABA in −S medium.

To determine whether the exclusive cytoplasmic localization of SnRK1A-GFP and MYBS1-GFP resulted from the cytoplasmic interaction between SKIN and SnRK1A in −S medium containing ABA, SnRK1A-GFP was transiently co-expressed with SKIN(Ri) in barley aleurones. SnRK1A-GFP was highly accumulated in the nucleus in the presence of SKIN(Ri) in −S medium regardless of the presence or absence of ABA (FIG. 10C and FIG. 22C). Transgenic rice overexpressing SKIN(Ri) was also transfected with SnRK1A-GFP and MYBS1-GFP. Similarly, SnRK1A-GFP and MYBS1-GFP became highly accumulated in the nucleus in −S medium despite the presence of ABA (FIG. 10D and FIG. 22D). These studies indicate that ABA promotes the cytoplasmic interaction between SKINs and SnRK1A as well as reduces the nuclear localization of SnRK1A and MYBS1.

### SKIN1 Hampers Seed Development by Repressing Enzymes Essential for Starch and GA Biosynthesis

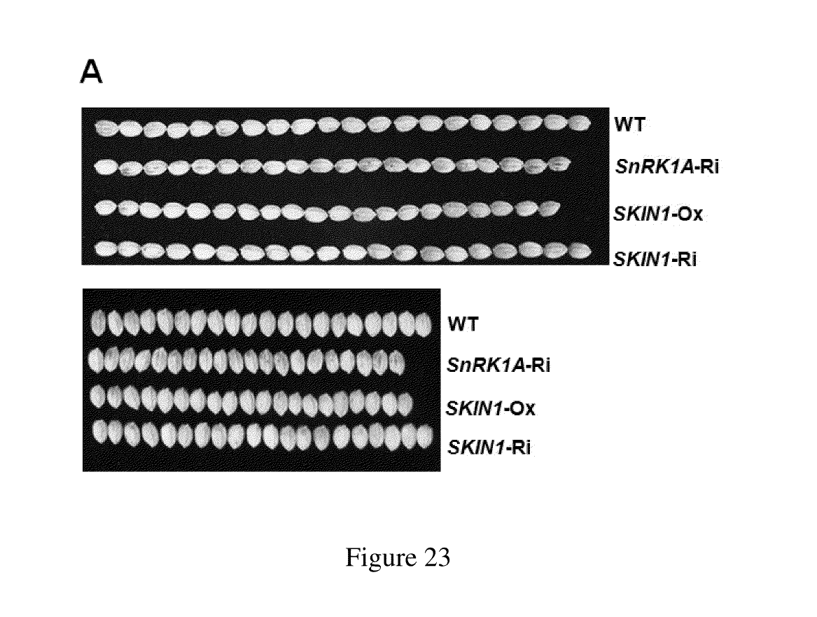

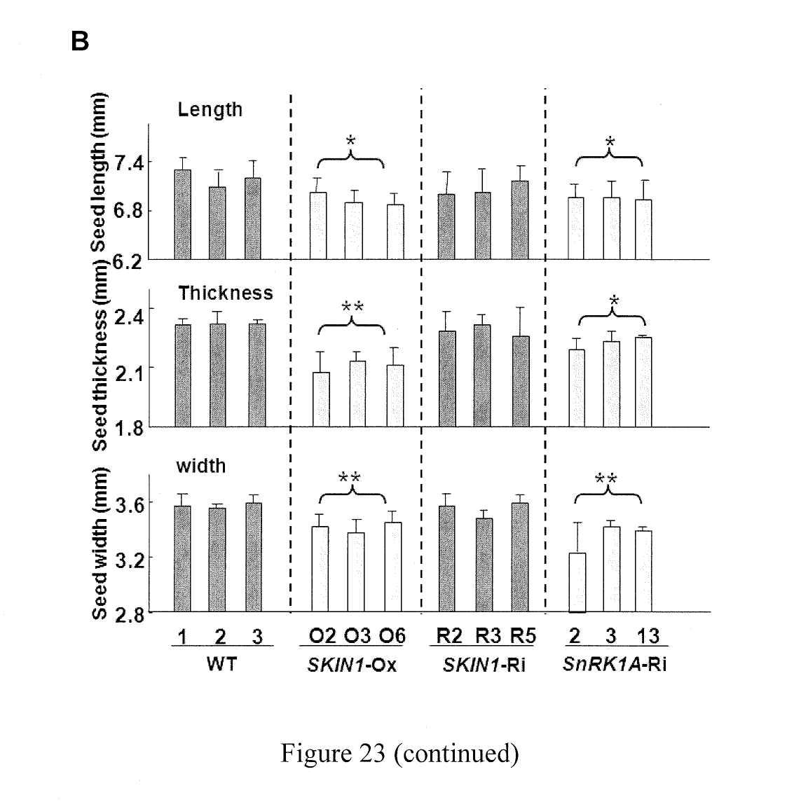

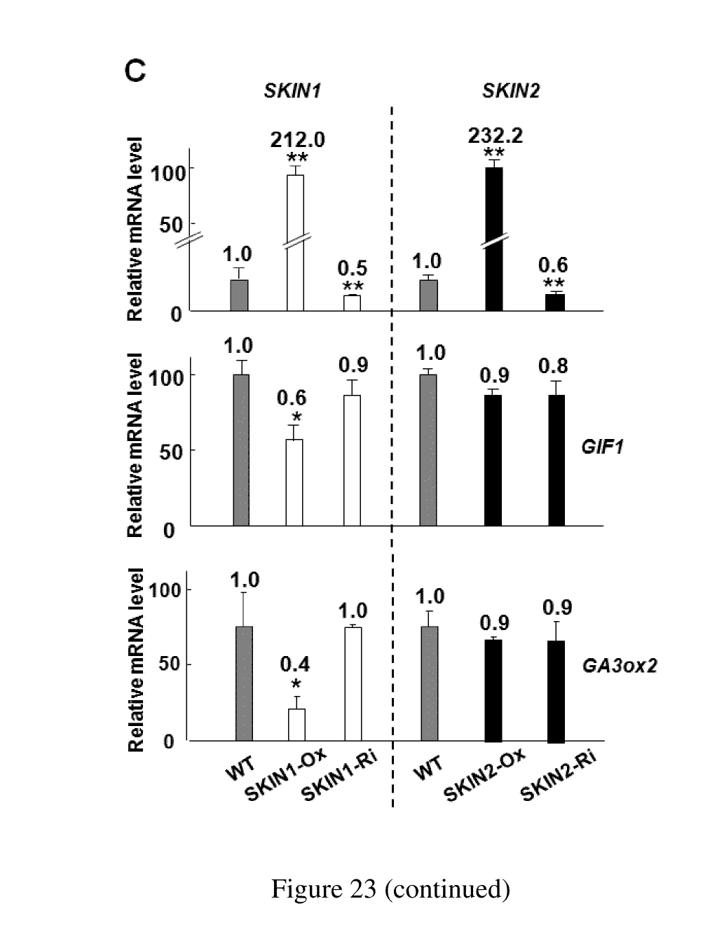

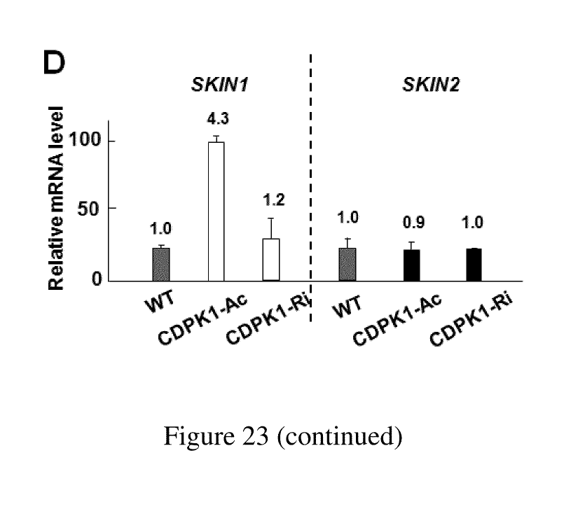

Since SnRK1s have been proposed to be involved in carbohydrate metabolism and starch biosynthesis (Polge and Thomas, 2007), the grain quality of SKIN1-Ox, SKIN1-Ri and SnRK1A-Ri transgenic lines were examined. The seed size of SKIN1-Ox and SnRK1A-Ri lines were smaller than the wild type (FIG. 23A). Quantitative analyses indicate that the seed length, thickness and width (FIG. 23B), and 1000-grain weight and grain yield (FIG. 23B) of SKIN1-Ox and SnRK1A-Ri lines were all significantly lower than the wild type and SKIN1-Ri lines.

GIF1 (Grain Incomplete Filling 1) gene, which encodes a cell-wall invertase (CIN2), is required for carbon partitioning during early grain-filling {Wang, 2008 #765}. By quantitative RT-PCR analysis, we found that the level of GIF1 mRNA was also reduced by 40% in immature panicles of SKIN1-Ox transgenic lines (FIG. 23C). Recently, we found a constitutively active calcium-dependent protein kinase 1 (CDPK1-Ac) represses the expression of GA3ox2, which is essential for GA biosynthesis, and reduces grain size in transgenic rice {Ho, 2013 #909}. We found that the level of GA3ox2 mRNA decreased by 60% in SKIN1-Ox transgenic lines (FIG. 23C). The expression of GIF1 was reduced by 20% but that of GA3ox2 was not altered in SnRK1A-Ri line, indicating the regulation of GA30x2 is SnRK1A-independent.

Taken together, these studies indicate that the grain development is hampered in plants with reduced SnRK1A activity, due to the elevated level of SKIN1 which represses the expression of enzymes essential for starch and GA biosynthesis.

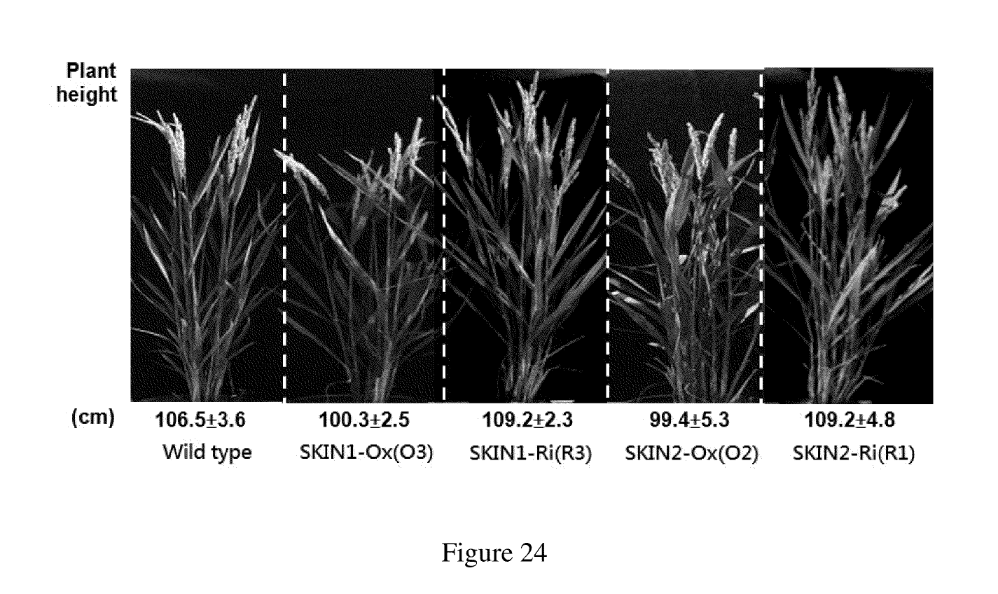

The height of SKIN-Ox mature plants in field conditions was only slightly reduced (FIG. 24). However, grain size, weight and yield were significantly reduced in SKIN1-Ox and SnRK1A-Ri plants plants (FIGS. 23A and 23B). Although SnRK1 has been shown to indirectly control carbohydrate metabolism through transcriptional regulation of enzymes involved in starch biosynthesis in potato tubers {Halford, 2003 #134; Polge, 2007 #356}, we were unable to detect altered accumulation of mRNAs encoding several enzymes potentially being involved in starch biosynthesis in developing rice seeds, such as starch branching enzyme I (BEI), isoamylase 1 (ISA1), starch synthase I (SSI, SSIIIa, SSIVa), granule-bound starch synthase (GBSSI), ADP-glucose pyrophosphorylase (AGPS2a, AGPS1, AGPL1), and sucrose synthase (Ss1, Ss2, Ss3) (data not shown).

In yeast, the SNF1 kinase complex is required for the transcriptional induction of glucose-repressible invertase for growth on sucrose as an alternative carbon source {Hardie, 1998 #129}. In plants, the cell wall invertase cleaves sucrose transported from source tissues into glucose and fructose that are then uptake by cells for starch biosynthesis in sink tissues and is proposed as a key enzyme in the source-sink regulation {Roitsch, 1999 #906}. GIF1 is a required for carbon partitioning during early grain-filling in rice, and gift mutant, although exhibits normal morphology and seed setting, has reduced grain weight {Wang, 2008 #765}. The present study demonstrates that GIF1 is regulated by the SnRK1A-dependant pathway in rice. GAs also regulate reproductive organ development, including both male and female flowers {King, 2003 #917}, and GA3ox2 is an essential enzyme for GA biosynthesis {Olszewski, 2002 #754}. SKIN1 may independently repress SnRK1A signaling and GA biosynthesis pathways due to following observations: First, the loss in grain yield was more significant in SKIN1-Ox lines than in SnRK1A-Ri lines (FIG. 23B). Second, GIF1 expression was reduced by 40% in SKIN1-Ox lines (FIG. 23C) but 20% in SnRK1A-Ri lines (FIG. 23D). Third, GA3ox2 was reduced in SKIN1-Ox lines (FIG. 23C) but not in SnRK1A-Ri lines (FIG. 23D).

SKINs are Novel Regulators Interacting with and Antagonizing the Function of SnRK1A

SKINs physically interact with SnRK1A in yeast and plant cells (FIG. 2 and FIG. 12). A few proteins interacting with SnRK1 have been identified in plants. For example, a PRL1 WD protein, which interacts with the two Arabidopsis SnRK1s (AKIN10 and AKIN11) in yeast, negatively regulates the activity of these two SnRK1s and downstream glucose-regulated genes in Arabidopsis (Bhalerao et al., 1999). A barley gene SnIP1 interacts with a seed-specific SnRK1 in vitro (Slocombe et al., 2002). Two proteins, PpSK11 and PpSK12, from the moss Physcomitrella patens interact with SnRK1 and inhibit its activity in yeast (Thelander et al., 2007). However, these proteins do not share homology with SKINs.

The KSD in SKINs is highly conserved in all SKIN homologs from monocots and dicots, and along with a conserved C-terminal NLS represent the most distinct signature of the SKIN closely-related family identified in five plant species (FIG. 1A). A few additional conserved domains are prominent in this protein family from monocots, suggesting distinct structural and/or functional features may exist between monocots and dicots. The function of KSD was not implicated in any member of the SKIN-related family previously, here we showed that the KSD was necessary for antagonism of the SnRK1A function (FIG. 3D). The N-terminal amino acids 1-83 and 1-85 of SKIN1 and SKIN2, respectively, interacted with the SnRK1A-KD in yeast and plant cells (FIG. 2 and FIG. 14); however, the KSD does not reside within these regions (FIG. 3D). It is unclear how SKIN-KSD interferes the SnRK1A function. A few domains are highly conserved in the N-terminus of SKINS, and some of them are also moncot-specific. The core domain in SKINs that interacts with the SnRK1A-KD remains to be better defined.

As far as we are aware of, the only member of this new protein family having been functionally studied is the Brassica BnKCP1, which is proposed as a transcription factor that interacts with the histone deacetylase HDA19 and activates cold-inducible genes in Arabidopsis (Gao et al., 2003). The KID in BnKCP1 is essential for interaction with HDA19 and shares some functional similarities with the KID in the mammalian cAMP-responsive element-binding (CREB) protein family (Gao et al., 2003). The typical KID composed of RRXS (where X means any amino acid) (Gonzalez et al., 1991) is conserved in both SKIN1 and SKIN 2 (RRAS), however, its relative position in the entire protein amino acid sequence is quite distinct from that in BnKCP1 (FIG. 1). Whether KID plays a function in the rice SKINs also remains to be determined.

Similar structural, functional and regulatory interactions among subunits in the SnRK1 complex observed in yeast also exist in plants (Lu et al., 2007; Polge and Thomas, 2007; Halford and Hey, 2009). In yeast, Snf1 is in the cytoplasm in glucose-containing medium but largely translocated into the nucleus with the assistance of Ga183 upon glucose starvation (Vincent et al., 2001), and Snf1-RD is responsible for the interaction with Ga183 (Jiang and Carlson, 1997). The detection of SnRK1A-RD in the nucleus in −S medium (FIG. 7) could be due to its lack of interactions with other cytoplasmic factors or efficient interactions with the rice Ga183 homolog. The high amount of cytoplasmic localization of SnRK1A-GFP was probably due to trapping by other cytoplasmic factors through the SnRK1A-KD or insufficient amount of endogenous Ga183 homolog for co-nuclear import (FIG. 7). Nevertheless, the accumulation of SnRK1A in the nucleus was increased significantly in cells in −S medium than in +S medium (FIG. 10A, panel 3).

The nuclear localization of Snf1 and SnRK1 has been shown to be essential for their protein kinase activities in yeast cells and Arabidopsis leaf mesophyll protoplasts, respectively (Vincent et al., 2001; Cho et al., 2012). It is unclear whether the nuclear localization of SnRK1A is essential for regulating the nutrient starvation signaling pathway. Previously, we showed that the expression of SnRK1A is induced by sugar starvation (Lu et al., 2007), therefore, the level of SnRK1A in the nucleus may be increased in −S medium. SKINs with or without NLSs maintained their antagonist activities (FIG. 8C), indicating that the antagonism of SKINs against SnRK1A is independent of its cellular localization. Without ABA, SnRK1A is absent in the nucleus under +S condition (FIG. 10A, panel 3), but present in both the nucleus and cytoplasm under −S condition (FIG. 10A, panel 7). Although SnRK1A significantly enhanced αAmy3 SRC promoter activity, the SRC activity was suppressed by SKINs to the background levels under −S condition (FIG. 3B and FIG. 8C). Consequently, the endogenous SnRK1A might be antagonized by SKINs in both the nucleus and cytoplasm.

The SnRK1A-Dependent Nutrient Starvation Signaling Pathway Plays a Key Role Regulating the Source-Sink Communication

SnRK1 has been shown to regulate similar physiological activities between moss and higher plants in terms of adaptation to limited energy. The double knockout mutant of two SnRK1 genes, snf1a and snf1b, of Physcomitrella patens has impaired capability to mobilize starch reserves in response to darkness, and can be kept alive only by feeding with glucose or providing constant light (Thelander et al., 2004). This mutant is unable to grow in a normal day (16 h)-night (8 h) cycle, presumably due to an inability to conduct normal carbohydrate metabolism under darkness (Thelander et al., 2004). Overexpression of two Arabidopsis SnRK1 s, KIN10 and KIN11, increases primary root growth under low light with limited energy, while the double kin10kin11 knockdown mutant, generated by virus-induced gene silencing, impairs starch mobilization from leaves at night and thus seedling growth (Baena-Gonzalez et al., 2007). Although SnRK1 has been proposed to regulate carbon partitioning between source and sink tissues in plants (Roitsch, 1999), the molecular and cellular mechanisms of its functions in source-sink communication are not well understood due to the inherent growth defects of snrk1-null mutants in higher plants.

In rice, the SnRK1 family has two members, SnRK1A/OSK1 and SnRK1B/OSK24 with amino acid sequences sharing 74% homology (Takano et al., 1998; Lu et al., 2007). Our previous studies demonstrated that SnRK1A, but not SnRK1B, mediating the sugar starvation signaling cascade in growing seedlings (Lu et al., 2007). SnRK1A is supposed to play a broader role in sugar regulation than SnRK1B, as SnRK1A is uniformly expressed in various growing tissues (including young roots and shoots, flowers and immature seeds) (Takano et al., 1998). SnRK1A functions upstream of MYBS1 and αAmy3 SRC, and plays a key role in regulating seed germination and seedling growth in rice (Lu et al., 2007). Expression of both SKINs could be detected in all tissues in seedlings, mature plants, flowers, and immature panicles (FIG. 19). These studies indicate that SnRK1A and SKINs are both expressed in germinating seeds and growing seedlings.

We showed that SKINs are sufficient and necessary for antagonism of SnRK1A function (FIG. 3B). Furthermore, in transgenic rice, the source-sink communication regulating nutrient mobilization in the endosperm during early seedling growth stages is found to act through the SnRK1A-dependent nutrient starvation signaling pathway. The expression of SKINs is induced by sugar starvation, similar to components in the sugar starvation signaling pathway (FIG. 4, panel 1). The accumulation of mRNA of MYBS1 and a variety of hydrolases was all suppressed in SKIN-Ox lines under +S and −S conditions, but only slightly increased in SKIN-Ri lines under +S but not under −S condition. SKIN1 and SKIN2 may have redundant functions, which lead to insignificant responses for enhancing endogenous gene expression in single-SKIN silenced lines under −S condition.

Seedling shoot and root growth was inhibited in SKIN-Ox plants but promoted in SKIN-Ri plants, and these effects were more evident in the dark, conditions that mimic sugar starvation, than in the light/dark cycle that produce sugars through photosynthesis (FIGS. 5A and 5B). The delay and promotion of seedling growth were accompanied by the decrease and increase in αAmy3 expression in SKIN-Ox and SKIN-Ri plants, respectively (FIG. 5C). Moreover, growth of SKIN-Ox seedlings could be recovered by the application of exogenous sugars. Similar negative effects of SKIN overexpression on seedling growth under hypoxia were also observed (FIG. 6). These studies indicate that the SnRK1A-dependent sugar demand signaling is necessary and sufficient for promoting sugar supply from the endosperm/aleuron (source), where hydrolases are produced for nutrient mobilization (FIGS. 4 and 5), to the germinated embryo/growing seedling (sink), where nutrients are utilized, and allows plants to grow under darkness or hypoxia. The expression of EP3A was regulated by SKINs similar to αAmy3 in seedlings (FIG. 5C), indicating that although required at less amounts, other nutrients likely are also coordinately produced through by the SnRK1A-regulated pathway.

### Differential Cellular Localization of Key Factors Regulates the Source-Sink Communication Under Abiotic Stresses

Plants are constantly exposed to environmental stresses, such as water deficit, flooding, extreme temperatures, and high salinity, that frequently inhibit photosynthesis, influence carbohydrate partitioning, constrain growth, and thus cause substantial yield loss. Several lines of evidences suggest that ABA might be a key signaling molecule regulating the SnRK1A-dependent sugar starvation signaling pathway via SKINs under abiotic stresses. First, the expression of SKINs was induced by various abiotic stresses and ABA (FIG. 9A). Second, ABA antagonizes the function of SnRK1A similarly to SKINs (FIG. 10). Third, ABA promotes the interaction between SnR1A and SKINs (FIG. 2D). Fourth, overexpression of SKINs promotes the ABA-mediated inhibition of seedling growth (FIG. 9B). The notion is further supported by the discovery that sugar starvation promotes whereas ABA inhibits the nuclear localization of SnRK1A (FIG. 10A, panel 3). Interestingly, SKINs were re-localized from the nucleus to cytoplasm, which was accompanied by the exclusion of SnRK1A and MYBS1 from the nucleus under −S condition in the presence of ABA (FIG. 10A, panels 5-8). The exclusion of SnRK1A from the nucleus was resulted from its interaction with SKINs in the cytoplasm, as the accumulation of SnRK1A in the nucleus was significantly enhanced by silencing of SKINs in barley aleurone cells transiently overexpressing SKIN(Ri) (compare FIG. 10C with FIG. 10A, panel 7) and in transgenic rice aleurone cells stably overexpressing SKIN(Ri) (FIG. 10D, compare panels 2 and 3 with panel 1) in −S condition with ABA treatment.

SnRK1 has been shown to regulate enzyme activity in the cytoplasm directly as well as act as a regulator of gene expression (Halford and Hey, 2009). SnRK1A seems to regulate the sugar starvation signaling pathway through various mechanisms. Previously, we showed that SnRK1A activates MYBS1 promoter activity and likely also phosphorylates MYBS1 directly (Lu et al., 2007). Additionally, the nuclear import of MYBS1 was inhibited by sugars and promoted by sugar starvation (FIG. 10B, panel 1) as has been reported previously (Hong et al., 2012). Here we further show that SnRK1A is sufficient and necessary for promoting the nuclear import of MYBS1 under +S and −S conditions, respectively (FIG. 10B, panels 2 and 3). However, as significant amounts of SnRK1A are localized in the cytoplasm as compared with the nucleus, it is unclear how MYBS1 is regulated by SnRK1A in the cytoplasm or nucleus. The recovery of nuclear localization of SnRK1A by SKIN silencing also recovered the nuclear enrichment of MYBS1 in transgenic rice under −S condition with ABA treatment (FIG. 10D, compare panels 5 and 6 with panel 4), indicating that the nuclear localization of SnRK1A and MYBS1 are tightly linked and suppressed by SKINs. It is conceivable that SKIN in the cytoplasm prevents the nuclear localization of SnRK1A and MYBS1, rendering them ineffective in up-regulating αAmy3 SRC activity.

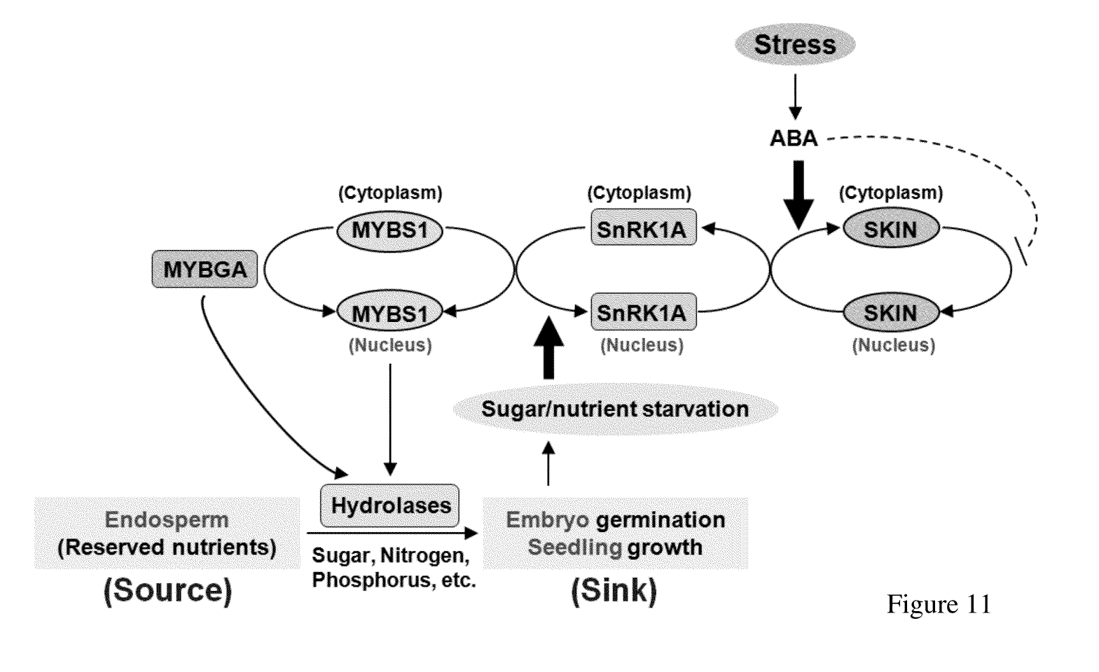

In summary, as illustrated in FIG. 11, the sink strength serves as a driving force and SnRK1A plays a central regulatory role in the source-sink communication. Differential cellular localization appears to be a key factor in this regulatory process. It has been demonstrated previously that the crucial GA regulator MYBGA facilitates the function and nuclear import of MYBS1 (Chen et al., 2006; Hong et al., 2012). Here, we further showed that sugar and nutrient demands, which are important signals from the sink tissue (germinating embryo and seedling), triggers the co-nuclear localization of two starvation signaling factors, i.e., SnRK1A and MYBS1, leading to the induction of α-amylase and other hydrolases necessary for the mobilization of nutrients in the source tissue (endosperm). Furthermore, stress and ABA not only induce the synthesis of SKIN, but also facilitate its exit from the nucleus to the cytoplasm or prevent its import from the cytoplasm to the nucleus. The cytoplasmic SKIN in turn binds to SnRK1A and prevents SnRK1A and MYBS1 from entering the nucleus, and eventually leading to the suppression of hydrolase production. However, since SnRK1A is highly accumulated in the cytoplasm even under sugar starvation, and SnRK1 protein kinase has substrates in the cytoplasm (Halford and Hey, 2009), the possibility that SnRK1A may also regulate the sugar starvation signaling pathway in the cytoplasm could not be ruled out. It is noted that SKIN is localized in the nucleus in the absence of ABA or stress, but function is unknown.

The current global climate changes tend to shift weather to more extreme perturbations, e.g., high and low temperatures, flooding, and water scarcity, which aggravate the world crop productivity that has already plateaued (IRRI, 2010). As the world population rises rapidly, development of crops that are more tolerant to various abiotic stresses while maintaining yield potentials remains an important and challenging task. In plants, SnRK1s regulate many aspects of growth and development during vegetative and reproductive stages (Polge and Thomas, 2007). To alleviate the negative effect of SKIN overexpression on plant growth, understanding the mode of action of SKINs on the restriction of plant growth temporally and spatially under abiotic stresses may facilitate the improvement of cereals with enhanced tolerance to abiotic stresses without yield penalty.

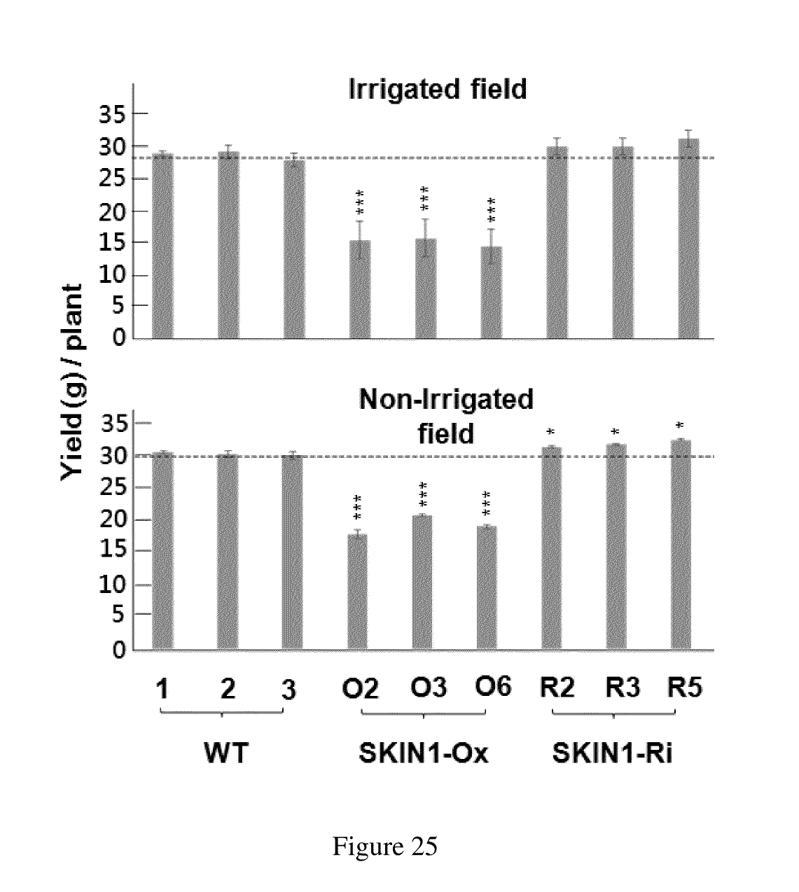

Wild type rice (WT) and SKIN1-Ox and SKIN1-Ri transgenic rice grew in irrigated field or non-irrigated field of National Chung Hsing University, Taiwan. In the first season of 2013, the climate and typhoon brought much rain, and the non-irrigated field was not as dry as expected. However, FIG. 25 shows that SKIN1-Ri transgenic rice increased the yield of rice by approximately 7.4% even if the conditions of non-irrigated field were not perfect. It proves that decreasing the expression of endogenous SKIN increases the yield of rice. If the conditions of non-irrigated field are good, the yield difference will be greater.

### REFERENCE

Akazawa, T., and Hara-Nishimura, I. (1985). Topographic aspects of biosynthesis, extracellular secretion and intracellular storage of proteins in plant cells. Annu Rev Plant Physiol 70, 441-472.
Baena-Gonzalez, E., Rolland, F., Thevelein, J. M., and Sheen, J. (2007). A central integrator of transcription networks in plant stress and energy signalling. Nature 448, 938-942.
Beck, E., and Ziegler, P. (1989). Biosynthesis and degradation of starch in higher plants. Annu Rev Plant Physiol Plant Mol Biol 40, 95-117.
Bhalerao, R. P., Salchert, K., Bako, L., Okresz, L., Szabados, L., Muranaka, T., Machida, Y., Schell, J., and Koncz, C. (1999). Regulatory interaction of PRL1 WD protein with Arabidopsis SNF1-like protein kinases. Proc Natl Acad Sci USA 96, 5322-5327.
Breeden, L., and Nasmyth, K. (1985). Regulation of the yeast HO gene. Cold Spring Harbor symposia on quantitative biology 50, 643-650.
Bruce, W. B., Christensen, A. H., Klein, T., Fromm, M., and Quail, P. H. (1989). Photoregulation of a phytochrome gene promoter from oat transferred into rice by particle bombardment. Proc Natl Acad Sci USA 86, 9692-9696.
Chan, M. T., and Yu, S. M. (1998). The 3′ untranslated region of a rice alpha-amylase gene functions as a sugar-dependent mRNA stability determinant. Proc Natl Acad Sci USA 95, 6543-6547.
Chen, P.-W., Lu, C.-A., Yu, T.-S., Tseng, T.-H., Wang, C.-S., and Yu, S.-M. (2002). Rice alpha-amylase transcriptional enhancers direct multiple mode regulation of promoters in transgenic rice. J Biol Chem 277, 13641-13649.
Chen, P. W., Chiang, C. M., Tseng, T. H., and Yu, S. M. (2006). Interaction between rice MYBGA and the gibberellin response element controls tissue-specific sugar sensitivity of alpha-amylase genes. Plant Cell 18, 2326-2340.
Chien, C. T., Bartel, P. L., Sternglanz, R., and Fields, S. (1991). The two-hybrid system: a method to identify and clone genes for proteins that interact with a protein of interest. Proc Natl Acad Sci USA 88, 9578-9582.
Cho, Y. H., Hong, J. W., Kim, E. C., and Yoo, S. D. (2012). Regulatory functions of SnRK1 in stress-responsive gene expression and in plant growth and development. Plant Physiol 158, 1955-1964.
Christensen, A. H., and Quail, P. H. (1996). Ubiquitin promoter-based vectors for high-level expression of selectable and/or screenable marker genes in monocotyledonous plants. Transgenic Res 5, 213-218.
Crute, B. E., Seefeld, K., Gamble, J., Kemp, B. E., and Witters, L. A. (1998). Functional domains of the alpha1 catalytic subunit of the AMP-activated protein kinase. J Biol Chem 273, 35347-35354.
Dyck, J. R., Gao, G., Widmer, J., Stapleton, D., Fernandez, C. S., Kemp, B. E., and Witters, L. A. (1996). Regulation of 5′-AMP-activated protein kinase activity by the noncatalytic beta and gamma subunits. J Biol Chem 271, 17798-17803.
Fincher, G. G. (1989). Molecular and cellular biology associated with endosperm mobilisation in germinating cereal grains. Ann Rev Plant Physiol Plant Mol Biol 40, 305-346.
Gao, M. J., Schafer, U. A., Parkin, I. A., Hegedus, D. D., Lydiate, D. J., and Hannoufa, A. (2003). A novel protein from Brassica napus has a putative KID domain and responds to low temperature. Plant J 33, 1073-1086.
Gomez-Cadenas, A., Zentella, R., Walker-Simmons, M. K., and Ho, T. H. (2001). Gibberellin/abscisic acid antagonism in barley aleurone cells: site of action of the protein kinase PKABA1 in relation to gibberellin signaling molecules. Plant Cell 13, 667-679.
Gonzalez, G. A., Menzel, P., Leonard, J., Fischer, W. H., and Montminy, M. R. (1991). Characterization of motifs which are critical for activity of the cyclic AMP-responsive transcription factor CREB. Mol Cell Biol 11, 1306-1312.
Gubler, F., Kalla, R., Roberts, J. K., and Jacobsen, J. V. (1995). Gibberellin-regulated expression of a myb gene in barley aleurone cells: evidence for Myb transactivation of a high-pI alpha-amylase gene promoter. Plant Cell 7, 1879-1891.
Gubler, F., Raventos, D., Keys, M., Watts, R., Mundy, J., and Jacobsen, J. V. (1999). Target genes and regulatory domains of the GAMYB transcriptional activator in cereal aleurone. Plant J 17, 1-9.
Halford, N. G., and Hey, S. J. (2009). Snf1-related protein kinases (SnRKs) act within an intricate network that links metabolic and stress signalling in plants. Biochem J 419, 247-259.
Halford, N. G., Hey, S., Jhurreea, D., Laurie, S., McKibbin, R. S., Paul, M., and Zhang, Y. (2003). Metabolic signalling and carbon partitioning: role of Snf1-related (SnRK1) protein kinase. J Exp Bot 54, 467-475.
Hardie, D. G., and Sakamoto, K. (2006). AMPK: a key sensor of fuel and energy status in skeletal muscle. Physiology (Bethesda) 21, 48-60.
Ho, S. L., Tong, W. F., and Yu, S. M. (2000). Multiple mode regulation of a cysteine proteinase gene expression in rice. Plant Physiol 122, 57-66.
Hong, Y. F., Ho, T. H., Wu, C. F., Ho, S. L., Yeh, R. H., Lu, C. A., Chen, P. W., Yu, L. C., Chao, A., and Yu, S. M. (2012). Convergent starvation signals and hormone crosstalk in regulating nutrient mobilization upon germination in cereals. Plant Cell 24, 2857-2873.
IRRI. (2010). Rice Policy—Why is it happening? http://beta.irri.org/solutions/index.php?option=com content&task=view&id=15.
Jiang, R., and Carlson, M. (1996). Glucose regulates protein interactions within the yeast SNF1 protein kinase complex. Genes Dev 10, 3105-3115.
Jiang, R., and Carlson, M. (1997). The Snf1 protein kinase and its activating subunit, Snf4, interact with distinct domains of the Sip1/Sip2/Ga183 component in the kinase complex. Mol Cell Biol 17, 2099-2106.
Kennedy, B. M. (1980). Nutritional quality of rice endosperm. Chapter 11, In: Rice: Production and Utilization. B. S. Luh ed., AVI Publishing Co., Westport, Conn. p. 439-469.
Lee, K. W., Chen, P. W., Lu, C. A., Chen, S., Ho, T. H., and Yu, S. M. (2009). Coordinated responses to oxygen and sugar deficiency allow rice seedlings to tolerate flooding. Sci Signal 2, ra61.
Lu, C. A., Lim, E. K., and Yu, S. M. (1998). Sugar response sequence in the promoter of a rice alpha-amylase gene serves as a transcriptional enhancer. J Biol Chem 273, 10120-10131.
Lu, C. A., Ho, T. H., Ho, S. L., and Yu, S. M. (2002). Three novel MYB proteins with one DNA binding repeat mediate sugar and hormone regulation of alpha-amylase gene expression. Plant Cell 14, 1963-1980.
Lu, C. A., Lin, C. C., Lee, K. W., Chen, J. L., Huang, L. F., Ho, S. L., Liu, H. J., Hsing, Y. I., and Yu, S. M. (2007). The SnRK1A protein kinase plays a key role in sugar signaling during germination and seedling growth of rice. Plant Cell 19, 2484-2499.
McKibbin, R. S., Muttucumaru, N., Paul, M. J., Powers, S. J., Burrell, M. M., Coates, S., Purcell, P. C., Tiessen, A., Geigenberger, P., and Halford, N. G. (2006). Production of high-starch, low-glucose potatoes through over-expression of the metabolic regulator SnRK1. Plant Biotechnol J 4, 409-418.
Polge, C., and Thomas, M. (2007). SNF1/AMPK/SnR1 kinases, global regulators at the heart of energy control? Trends Plant Sci 12, 20-28.
Purcell, P. C., Smith, A. M., and Halford, N. G. (1998). Antisense expression of a sucrose non-fermenting-1-related protein kinase sequence in potato results in decreased expression of sucrose synthase in tubers and loss of sucrose-inducibility of sucrose synthase transcripts in leaves. Plant-J. 14, 195-202.
Radchuk, R., Emery, R. J., Weier, D., Vigeolas, H., Geigenberger, P., Lunn, J. E., Feil, R., Weschke, W., and Weber, H. (2010). Sucrose non-fermenting kinase 1 (SnRK1) coordinates metabolic and hormonal signals during pea cotyledon growth and differentiation. Plant J 61, 324-338.
Rogers, J. C., Lanahan, M. B., and Rogers, S. W. (1994). The cis-acting gibberellin response complex in high pI alpha-amylase gene promoters. Requirement of a coupling element for high-level transcription. Plant Physiol 105, 151-158.
Roitsch, T. (1999). Source-sink regulation by sugar and stress. Curr Opin Plant Biol 2, 198-206.
Rolland, F., Baena-Gonzalez, E., and Sheen, J. (2006). Sugar sensing and signaling in plants: Conserved and Novel Mechanisms. Annu Rev Plant Biol 57, 675-709.
Sheu, J.-J., Jan, S.-P., Lee, H.-T., and Yu, S.-M. (1994). Control of transcription and mRNA turnover as mechanisms of metabolic repression of alpha-amylase gene expression. Plant J 5, 655-664.
Sheu, J.-J., Yu, T.-S., Tong, W.-F., and Yu, S.-M. (1996). Carbohydrate starvation stimulates differential expression of rice alpha-amylase genes that is modulated through complicated transcriptional and posttranscriptional processes. J Biol Chem 271, 26998-27004.
Slocombe, S. P., Laurie, S., Bertini, L., Beaudoin, F., Dickinson, J. R., and Halford, N. G. (2002). Identification of SnIP1, a novel protein that interacts with SNF1-related protein kinase (SnRK1). Plant Mol Biol 49, 31-44.
Sun, T. p., and Gubler, F. (2004). Molecular mechanism of gibberellin signaling in plants. Annu Rev Plant Biol 55, 197-223.
Takano, M., Kajiya-Kanegae, H., Funatsuki, H., and Kikuchi, S. (1998). Rice has two distinct classes of protein kinase genes related to SNF1 of Saccharomyces cerevisiae, which are differently regulated in early seed development. Mol Gen Genet 260, 388-394.
Thelander, M., Olsson, T., and Ronne, H. (2004). Snf1-related protein kinase 1 is needed for growth in a normal day-night light cycle. EMBO J 23, 1900-1910.
Thelander, M., Nilsson, A., Olsson, T., Johansson, M., Girod, P. A., Schaefer, D. G., Zryd, J. P., and Ronne, H. (2007). The moss genes PpSKI1 and PpSKI2 encode nuclear SnRK1 interacting proteins with homologues in vascular plants. Plant Mol Biol 64, 559-573.
Vincent, O., Townley, R., Kuchin, S., and Carlson, M. (2001). Subcellular localization of the Snf1 kinase is regulated by specific beta subunits and a novel glucose signaling mechanism. Genes Dev 15, 1104-1114.
Woodger, F., Jacobsen, J. V., and Gubler, F. (2004). Gibberellin action in germinated cereal grains In: Plant Hormones: Biosynthesis, signal Transduction, Action!, (Ed.) P. J. Davies. Kluwer Academic Publishers, Dordrecht. p. 221-240.
Yu, S. M. (1999a). Regulation of alpha-amylase gene expression. In Molecular Biology of Rice, K. Shimamoto, ed (Springer-Verlag, Tokyo), pp. 161-178.
Yu, S. M. (1999b). Cellular and genetic responses of plants to sugar starvation. Plant Physiol 121, 687-693.
Zhang, Y., Shewry, P. R., Jones, H., Barcelo, P., Lazzeri, P. A., and Halford, N. G. (2001).
Expression of antisense SnRK1 protein kinase sequence causes abnormal pollen development and male sterility in transgenic barley. Plant J 28, 431-441.

What is claimed is:

1. A SKIN gene silencing plasmid, comprising a promoter; two DNA fragments, which are obtained from one DNA fragment derived from the cDNA of SKIN1 or SKIN2 and arranged in sense and antisense orientation; and a third DNA fragment inserted between the two DNA fragments.

2. The SKIN gene silencing plasmid according to claim 1, wherein the one DNA fragment derived from the cDNA of SKIN1 is SEQ ID No: 58.

3. The SKIN gene silencing plasmid according to claim 1, wherein the one DNA fragment derived from the cDNA of SKIN2 is SEQ ID No: 59.

4. The SKIN gene silencing plasmid according to claim 1, wherein the third DNA sequence is SEQ ID No: 60.

5. The SKIN gene silencing plasmid according to claim 1, wherein the promoter is selected from 35CaMV, actin1, G1uB1, rbcS, cab, SNAC1, pin2, SAG12, Psam1, TobRB7 or ubiquitin promoter.

6. A transformed plant cell, comprising the SKIN gene silencing plasmid according to claim 1.

7. The transformed plant cell according to claim 6, wherein the one DNA fragment derived from the cDNA of SKIN1 is SEQ ID No: 58.

8. The transformed plant cell according to claim 6, wherein the one DNA fragment derived from the cDNA of SKIN2 is SEQ ID No: 59.

9. The transformed plant cell according to claim 6, wherein the third DNA sequence is SEQ ID No: 60.

10. The transformed plant cell according to claim 6, wherein the promoter is selected from 35CaMV, actin1, G1uB1, rbcS, cab, SNAC1, pin2, SAG12, Psam1, TobRB7 or ubiquitin promoter.

11. The transformed plant cell according to claim 6, wherein the plant is a monocot selected from maize, wheat, barley, millet, sugarcane, Miscanthus, switchgrass or sorghum.

12. The transformed plant cell according to claim 6, wherein the plant is a dicot selected from Arabidopsis, tomato, potato, brassica, soybean, canola or sugarbeet.

13. A transgenic plant, comprising the SKIN gene silencing plasmid according to claim 1.

14. The transgenic plant according to claim 15, wherein the one DNA fragment derived from the cDNA of SKIN1 is SEQ ID No: 58.

15. The transgenic plant according to claim 15, wherein the one DNA fragment derived from the cDNA of SKIN2 is SEQ ID No: 59.

16. The transgenic plant according to claim 15, wherein the third DNA sequence is SEQ ID No: 60.

17. The transgenic plant according to claim 15, wherein the promoter is selected from 35CaMV, actin1, G1uB1, rbcS, cab, SNAC1, pin2, SAG12, Psam1, TobRB7 or ubiquitin promoter.

18. The transgenic plant according to claim 15, wherein the plant is a monocot selected from maize, wheat, barley, millet, sugarcane, Miscanthus, switchgrass or sorghum.

19. The transgenic plant according to claim 15, wherein the plant is a dicot selected from Arabidopsis, tomato, potato, brassica, soybean, canola or sugarbeet.
